(19) United States
(12) Patent Application Publication
(10) Pub. No.: US 2024/0120053 A1
(43) Pub. Date: Apr. 11, 2024

(54) 5-HT RECEPTOR AGONIST TITRATION

(71) Applicant: Diamond Therapeutics Inc., Toronto (CA)

(72) Inventors: Judith BLUMSTOCK, Toronto (CA); William James TYLER, Chelsea, AL (US); Jeffrey SPROUSE, Stonington, CT (US)

(21) Appl. No.: 18/461,776

(22) Filed: Sep. 6, 2023

Related U.S. Application Data
(63) Continuation of application No. PCT/IB2022/000103, filed on Mar. 7, 2022.
(60) Provisional application No. 63/158,245, filed on Mar. 8, 2021.
(60) Provisional application No. 63/297,668, filed on Jan. 7, 2022.

(51) Int. Cl. G16H 20/10; A61B 5/00; A61B 5/378; A61K 31/675; A61P 25/28; G16H 10/20

(52) U.S. Cl. CPC G16H 20/10; A61B 5/378; A61B 5/4848; A61K 31/675; A61P 25/28; G16H 10/20

(57) ABSTRACT

Disclosed herein are methods of treating neuropsychiatric and cognitive conditions in individuals by titrating a 5-HT receptor agonist over a period of time to provide a therapeutic effective amount of the 5-HT receptor agonist to the individual.

### CROSS-REFERENCE

This application is continuation of International Application Number PCT/182022/000103, filed Mar. 7, 2022, which claims benefit of U.S. Provisional Patent Application No. 63/158,245 filed on Mar. 8, 2021, and U.S. Provisional Patent Application No. 63/297,668 filed on Jan. 7, 2022, each of which is incorporated herein by reference in its entirety.

### BACKGROUND

The serotonin (5-HT) receptors are a group of G protein-coupled receptors (GPCRs) and ligand-gated ion channels found in the central and peripheral nervous systems. Activation of 5-HT receptors can substantially influence brain function.

### SUMMARY

Provided in certain embodiments herein is a method for treating a condition treatable with a 5HT receptor agonist. In some embodiments, provided herein is a method for treating a mental, a behavioral, or a neuropsychiatric condition (e.g., a symptom thereof) in an individual. In some embodiments, provided herein is a method of identifying a therapeutically effective amount or dose of a therapeutic agent, such as for use in a method for treating a mental, a behavioral, or a neuropsychiatric condition in an individual. In some instances, methods provided herein are useful for providing personalized care in a particular individual, such as determining a specific therapeutically effective dose for that particular individual. Further, in some embodiments, methods provided herein are useful in maintaining a desired effect of a therapeutic agent provided herein, such as by monitoring the effects of a therapy and adjusting the dose as the effects of the therapy change. For example, in some instances, the therapeutic effect of a certain dose of a therapeutic agent may diminish over time; in certain instances, methods provided herein facilitate identification of a time at which a therapeutic effect of a particular dose is diminished to such a point as to warrant an increase in dose. In other examples, the therapeutic effect of a dose may vary depending on different metabolic or other states of an individual on a particular day or week; in some instances, methods provided herein facilitate identification of a time at which a therapeutic effect of a particular dose may not be sufficient to produce a desired effect and may warrant an increase in dose, or facilitate identification of a time at which an undesired effect (e.g., hallucination) of a particular dose may warrant a decrease in the desired therapeutic dose.

In some embodiments, provided herein is a method for identifying a (e.g., personalized) therapeutically effective dose of an agent (e.g., a 5HT agonist, such as psilocybin) in an individual (e.g., a particular individual). In some embodiments, the method comprises (1) administering to an individual a first amount of a therapeutic agent (e.g., a 5-hydroxytryptamine (5-HT) receptor agonist (e.g., psilocybin)); and (2) identifying or measuring a first state (e.g., a physiological parameter, such as brain activity) of the individual by performing an (e.g., physiological, such as brain) evaluation (e.g., an electroencephalogram (EEG)) on the individual. In some embodiments, a therapeutically effective dose of the agent is determined (at least in part) by the identification or measurement of the (e.g., physiological, such as brain) evaluation of step (2).

In some embodiments, provided herein is a method for identifying a (e.g., personalized) therapeutically effective dose of an agent (e.g., a 5HT agonist, such as psilocybin) in an individual (e.g., a particular individual). In some embodiments, the method comprises (1) identifying or measuring a first state (e.g., a physiological parameter, such as brain activity) of the individual by performing an (e.g., physiological, such as brain) evaluation (e.g., an electroencephalogram (EEG)) on the individual; (2) administering to an individual a first amount of a therapeutic agent (e.g., a 5-hydroxytryptamine (5-HT) receptor agonist (e.g., psilocybin)); and (3) identifying or measuring a second state (e.g., a physiological parameter, such as brain activity) of the individual by performing a second (e.g., physiological, such as brain) evaluation (e.g., an electroencephalogram (EEG)) on the individual (e.g., wherein the first and second evaluation are of the same type, such that the evaluated outputs can be compared to one another to determine a change). In some embodiments, a therapeutically effective dose of the agent is determined (at least in part) by the identification or measurement of the evaluation of step (3). In certain embodiments, the method comprises comparing (e.g., determining the difference between) the first state and second state (e.g., measurements thereof). In some embodiments, a therapeutically effective dose of the agent is determined (at least in part) based on the comparison (e.g., difference) between the first and second states (e.g., physiological parameter, such as brain activities) (e.g., measurements thereof).

In certain embodiments, the state of an individual can be a physiological, neurological, psychological, metabolic, or biological state or a combination thereof. In some embodiments the state of an individual is measured or identified by a brain activity (e.g. using a brain evaluation (e.g. EEG, MEG, fNRIS, PET transcranial functional ultrasound)), a heart test (e.g. evaluated using an EEG for recording heart rate or EKG for recording heart rhythm), a visual test, an auditory test, a biological sample (e.g. for evaluating changes in serum, plasma, whole blood, urine, sweat or the like), patient reporting (e.g. through questionnaires or surveys to evaluate mood, affect, coping, sleep quality, stress, anxiety, memory, and/or other emotions), functional data (e.g. evaluated by brain imaging (e.g. fNRIS, PET) or monitoring of brain activity levels (e.g. EEG)), body temperature, food intake, metabolic rate, perspiration, hydration, salivation, pupil dilation, breathing rate, puke rate, skin color, or skin temperature, and the like.

In some embodiments, the state of the individual may be measured or identified by evaluating one or more of EEG, EKG, MEG, fNIRS, PET, transcranial functional ultrasound, visual stimuli (e.g. colour, shape, pattern, emotional face, video, flash, milli second or longer), auditory stimuli (e.g. matched paired, acoustic frequency, Hz, milli second or longer), ultrasound waves by a sensor e.g., a piezoelectric resonant material (e.g., a PZT, a CMUT, a PMUT), clinical effect (visual, auditory, body, time and space, cognition, drowsiness, confusion, impairment), standard/deviant waveforms, evoked power/potential, voltage (alpha bands, beta bands, gamma bands, delta bands, or theta bands), asynchronization, time-locked, magnetic, hemodynamic (flux, flow, velocity, oxygenation), MMN, ASSR, surveys including 5D-ASC, Beck Depression, Coping, DASS42, Gad-7 Anxiety, PANAS-GEN, SF-36 QOL, STAI 6, brain imaging tests, heart rate, cardiovascular activity, photodiode, skin conduction and impedance, biological samples such as serum, plasma, whole blood, urine, sweat or the like, visual perception alteration, an auditory perception alteration, bodily perception alteration, a temporal perception alteration, or a spatial perception alteration, sleep quality, patient reported outcomes (e.g. electronic) or subjective mood, affect, coping, sleep quality, stress, anxiety, memory, and other emotional or functional data with brain imaging or brain activity, providing data (e.g., patterns, clusters, classifications, amplitudes, frequencies, or magnitude) patient evaluation (e.g., physicians, counselors, psychologist, or spiritual leader), data comprising visual representations of brain responses or brain activity data and/or survey or patient reported outcomes to help convey the effectiveness or lack thereof of a psychedelic therapy, training modules, machine learning algorithms and training sets, artificial intelligence (e.g., deep convolution neural networks) algorithms and/or machine learning processes to classify, cluster, or recognize patterns or relationships amongst sensory-evoked (e.g., auditory or visual stimuli) brain activity or resting state brain activity acquired by brain imaging methods and patient reported outcomes (e.g., changes in mood, anxiety, motivation, or memory), auditory brainstem responses (ABR), paired pulse inhibition (PPI; ie P50 auditory suppression; sensory gating), auditory mismatch negativity (MNN), and auditory steady state responses (ASSR), if EEG measures of auditory sensory evoked activity, cognitive control of attention and emotion, sub-thalamic and cortical levels, amplitudes of MMN potentials will be correlated to depression, anxiety, and mood survey data, Emotional Flanker Task, Erikson Flanker Task, Continuous Performance Test (CPT), Connor's CPT, oddball tasks, State Trait Anxiety Inventory is a 6-item, self-report survey, sleep diaries, the symmetry and power of alpha, beta, delta, and gamma brainwave activity across prefrontal cortex brain regions, Medical Quality of Life Outcomes Study 36-Item Short Form Health Survey (SF-36) and Physical Component Summary (PCS) and Mental Component Summary (MCS) scores, physical functioning, role physical, role emotional, bodily pain, vitality, social functioning, mental health, and general health on Likert, salivary cytokines, salivary brain-derived neurotrophic factors, psychophysical testing, assessing congruent, incongruent, and neutral conditions, surveys assessing mood, depression, anxiety, and stress, Mood Circumplex, DSM-V, cognitive behavioral therapy, synergy score, serenity score, quality of life, Sleep diary and Mood Capture, questions about attention, social interactions, fatigue and mood, approach avoid metric, biometric of coping, HRV spot check, or camera.

In some embodiments, the methods of the present application include measuring and identifying a state of an individual for the purposes of diagnosis, monitoring (e.g., feedback) or treatment.

In certain embodiments, provided herein is a method for diagnosing, monitoring or treating an individual (e.g., in need thereof), the method comprising:
a. identifying or measuring a state of the individual by performing an evaluation (e.g., of the state of the individual);
b. administering to the individual a first amount of one or more 5-hydroxytryptamine (5-HT) receptor agonist (e.g., psilocybin), or a pharmaceutically acceptable salt, solvate, metabolite, derivative, or prodrug thereof;
c. identifying or measuring a second state of the individual by performing another evaluation (e.g., of the state of the individual) subsequent to administering the first amount of the one or more 5-HT receptor agonist (e.g., psilocybin), or a pharmaceutically acceptable salt, solvate, metabolite, derivative, or prodrug thereof; and
d. optionally, administering to the individual a second amount of one or more 5-HT receptor agonist (e.g., psilocybin), or a pharmaceutically acceptable salt, solvate, metabolite, derivative, or prodrug thereof.

In certain embodiments, provided herein is a method of treating a mental, a behavioral, or a neuropsychiatric condition (e.g., symptom thereof) in an individual (e.g., in need thereof), the method comprising (1) identifying a therapeutic dose according to a process described above; and (2) administering to the individual the therapeutically effective dose. In some embodiments, provided herein is a method of treating a mental, a behavioral, or a neuropsychiatric condition (e.g., symptom thereof) in an individual (e.g., in need thereof), the method comprising administering to the individual a therapeutically effective dose, the therapeutic dose having been identified according to a process herein, such as described above. In certain embodiments, the method of identifying the therapeutic dose is repeated (such as daily, weekly, or the like), such as to provide an updated, accurate, current, and/or personalized therapeutic dose for the individual, such as on an ongoing basis.

In certain embodiments, provided herein is a method for treating a condition (e.g., a mental, a behavioral, or a neuropsychiatric condition) treatable with a 5-HT receptor agonist, and/or symptoms thereof, in an individual (e.g., in need thereof), comprising:
a) identifying or measuring a first state (e.g., a physiological parameter, such as brain activity) of the individual by performing an (e.g., physiological, such as brain) evaluation (e.g., an electroencephalogram (EEG)) on the individual;
b) administering to the individual a first amount of one or more 5-hydroxytryptamine (5-HT) receptor agonist (e.g., psilocybin), or a pharmaceutically acceptable salt, solvate, metabolite, derivative, or prodrug thereof;
c) identifying or measuring a second state (e.g., a physiological parameter, such as brain activity) of the individual by performing a second (e.g., physiological, such as brain) evaluation (e.g., another electroencephalogram (EEG) on the individual) subsequent to administering the first amount of the one or more 5-HT receptor agonist (e.g., psilocybin), or a pharmaceutically acceptable salt, solvate, metabolite, derivative, or prodrug thereof; and
d) administering to the individual a second (e.g., therapeutically effective) amount of one or more 5-HT receptor agonist (e.g., psilocybin), or a pharmaceutically acceptable salt, solvate, metabolite, derivative, or prodrug thereof (e.g., thereby treating or managing the mental, the behavioral, or the neuropsychiatric condition, and/or the symptoms thereof, in the individual).

In some embodiments, the condition is a mental, a behavioral, or a neuropsychiatric condition. In some embodiments, the first state comprises a first brain activity and the second state comprises a second brain activity.

Provided in some embodiments herein are methods for identifying a dose (e.g., a therapeutically effective dose) of one or more 5-hydroxytryptamine (5-HT) receptor agonist (e.g., psilocybin) to be administered to an individual (e.g., in need thereof (e.g., in an individual susceptible to or suffering from a brain disorder or condition (e.g., a mental, a behavioral, a neuropsychiatric, or the like, condition))), such as, by identifying one or more (e.g., neurological) effect (e.g., one or more measurable neurological response) in the individual (e.g., in need thereof) (e.g., before, during, or subsequent to administering the one or more 5-HT receptor agonist (e.g., psilocybin)). In some embodiments, the dose (e.g., the therapeutically effective dose) of the one or more 5-HT receptor agonist (e.g., psilocybin) is a safe dose (e.g., a dose that does not produce an adverse event (e.g., hallucinations) in the individual). In some embodiments, the dose (e.g., the therapeutically effective dose) of one or more 5-HT receptor agonist (e.g., psilocybin) is identified by measuring one or more (e.g., neurological) effect (e.g., one or more measurable neurological response) in an individual, such as, by performing one or more brain evaluation (e.g., electroencephalogram (EEG)) on the individual (e.g., using a device capable of measuring one or more brain activity in the individual). In some embodiments, provided herein are methods for identifying or monitoring one or more (e.g., neurological) effect (e.g., one or more measurable neurological response) in an individual (e.g., in need thereof) administered a dose (e.g., a therapeutically effective dose) of one or more 5-HT receptor agonist (e.g., psilocybin). In some embodiments, the one or more effect (e.g., one or more measurable neurological response) is identified or monitored (in the individual) by performing one or more brain evaluation (e.g., electroencephalogram (EEG)) on the individual (e.g., using a device capable of measuring one or more brain activity in the individual), such as, subsequent to one or more 5-HT receptor agonist (e.g., psilocybin) being administered to the individual. In some embodiments, the one or more 5-HT receptor agonist (e.g., psilocybin) is administered to the individual in an amount sufficient to achieve a desired (e.g., neurological) effect (e.g., a desired neurological response) in the individual, such as, as measured by one or more brain evaluation (e.g., electroencephalogram (EEG)) performed on the individual. In some embodiments, provided herein are methods for titrating a dose (e.g., a therapeutically effective dose) of one or more 5-hydroxytryptamine (5-HT) receptor agonist (e.g., psilocybin) in an individual (e.g., in need thereof), such as, to achieve a desired (e.g., neurological) effect (e.g., a desired neurological response) in the individual, such as, as measured by one or more brain evaluation (e.g., electroencephalogram (EEG)) performed on the individual.

In some embodiments, the one or more brain evaluation is performed by the individual (e.g., at least once daily) (e.g., in or outside a healthcare setting (e.g., in a natural environment, such as, for example, at home, at work, or the like)). In some embodiments, the effect of the dose (e.g., the therapeutically effective dose) of the one or more 5-HT receptor agonist (e.g., psilocybin) is monitored (e.g., in the individual) by a heath care provider (e.g., outside of a healthcare setting) (e.g., using an application configured to receive data from the device capable of measuring one or more brain activity in the individual). In some embodiments, the therapeutically effective dose of the one or more 5-HT receptor agonist (e.g., psilocybin) is identified (in the individual) by a heath care provider (e.g., outside of a healthcare setting) (e.g., using an application configured to receive data from the device capable of measuring one or more brain activity in the individual).

In some embodiments, provided herein are methods for treating or reducing the incidence of an episode in an individual susceptible to or suffering from a brain disorder or condition (e.g., a mental, a behavioral, a neuropsychiatric, or the like, condition) by administering (e.g., titrating) a dose (e.g., a therapeutically effective dose) of one or more 5-HT receptor agonist (e.g., psilocybin) to the individual.

Provided in some embodiments herein is a method for treating or reducing the incidence of a brain disorder or condition, and/or symptoms thereof, in an individual (e.g., in need thereof).

Provided in some embodiments herein is a method for treating or reducing the incidence of a mental, a behavioral, or a neuropsychiatric condition, and/or symptoms thereof, in an individual (e.g., in need thereof).

Provided in some embodiments herein is a method for treating attention deficit hyperactivity disorder (ADHD) or attention deficit disorder (ADD) in an individual (e.g., in need thereof).

Provided in some embodiments herein is a method for treating mild cognitive impairment, dementia, or Alzheimer's disease in an individual (e.g., in need thereof).

Provided in some embodiments herein is a method for improving coping in an individual (e.g., in need thereof). In some embodiments, coping is psychological coping. In some embodiments, the method is for improving stress recovery rate in the individual. In some embodiments, the individual is suffering from or susceptible to stress.

Provided in some embodiments herein is a method for increasing motivation in an individual (e.g., in need thereof). In some embodiments, the individual is suffering from or susceptible to low motivation, anxiety, apathy, fear, phobia, constructive impulsivity, depression, or the like. In some embodiments, the low motivation is induced by stress, anxiety, or the like.

Provided in some embodiments herein is a method for treating addiction in an individual (e.g., in need thereof).

Provided in some embodiments herein is a method for treating brain inflammation (e.g., encephalitis) or brain fog in an individual (e.g., in need thereof). In some embodiments, the brain inflammation or brain fog is secondary to a concussion, traumatic brain injury (TBI) (e.g., mild TBI (mTBI)), or the like. In some embodiments, the brain inflammation or brain fog is secondary to a natural insult (e.g., injury, stroke, or the like). In some embodiments, the individual is suffering from or susceptible to brain inflammation (e.g., encephalitis) or brain fog.

In some embodiments, the method comprises identifying or measuring a first brain activity of the individual. In some embodiments, the first brain activity is identified or measured by performing a brain evaluation on the individual. In some embodiments, the first brain activity is an initial (e.g., a baseline) brain activity. In some embodiments, the first brain activity is an initial (e.g., a baseline) brain activity that is performed prior to administering one or more 5-HT receptor agonist (e.g., psilocybin) to the individual. In some embodiments, the brain evaluation is a brain imaging test. In some embodiments, the brain evaluation is an electroencephalogram (EEG), a magnetoencephalogram (MEG), functional near-infrared spectroscopy (fNIRS), Positron Emission Tomography (PET), or a transcranial functional ultrasound imaging. In some embodiments, the brain evaluation is an EEG.

In some embodiments, the method comprises administering to the individual a first amount of one or more 5-hydroxytryptamine (5-HT) receptor agonist (e.g., psilocybin), or a pharmaceutically acceptable salt, solvate, metabolite, derivative, or prodrug thereof. In some embodiments, the first amount of the one or more 5-HT receptor agonist (e.g., psilocybin) is not a therapeutically effective amount. In some embodiments, the first amount of the one or more 5-HT receptor agonist (e.g., psilocybin) produces a significant change in brain activity (e.g., compared to a baseline brain activity) but does not produce a sustained or therapeutic effect in the individual. In some embodiments, the first amount of the one or more 5-HT receptor agonist (e.g., psilocybin) is a therapeutically effective amount.

In some embodiments, the method comprises identifying or measuring a second brain activity subsequent to administering the first amount of the one or more 5-HT receptor agonist (e.g., psilocybin), or a pharmaceutically acceptable salt, solvate, metabolite, derivative, or prodrug thereof. In some embodiments, the second brain activity is a lack of brain activity. In some embodiments, the second brain activity is identified or measured by performing another brain evaluation on the individual. In some embodiments, the other brain evaluation is a brain imaging test. In some embodiments, the other brain evaluation is an EEG, a MEG, PET, fNIRS, or a transcranial functional ultrasound imaging. In some embodiments, the other brain evaluation is an EEG.

In some embodiments, the method comprises administering to the individual a second amount of one or more 5-HT receptor agonist (e.g., psilocybin), or a pharmaceutically acceptable salt, solvate, metabolite, derivative, or prodrug thereof. In some embodiments, the second amount of the one or more 5-HT receptor agonist (e.g., psilocybin) is a therapeutically effective amount of the one or more 5-HT receptor agonist (e.g., psilocybin). In some embodiments, the first amount of the one or more 5-HT receptor agonist (e.g., psilocybin) produces a significant change in brain activity (e.g., compared to the first brain activity) but does not produce a sustained or therapeutic effect in the individual. In some embodiments, the second amount of the one or more 5-HT receptor agonist (e.g., psilocybin) is not a therapeutically effective amount. In some embodiments, a third amount of the one or more 5-HT receptor agonist (e.g., psilocybin), or a pharmaceutically acceptable salt, solvate, metabolite, derivative, or prodrug thereof, is administered to the individual, the third amount being selected to provide an improved (e.g., optimized) effect relative to the effect of the first amount and/or the second amount. In some embodiments, optimizing a dose of one or more 5-HT receptor agonist (e.g., psilocybin) comprises increasing, decreasing, or retaining the amount of the one or more 5-HT receptor agonist (e.g., psilocybin), or a pharmaceutically acceptable salt, solvate, metabolite, derivative, or prodrug thereof (e.g., compared to a first amount, a second amount, a third amount, or the like). In some embodiments, a first amount, a second amount, a third amount, or the like, are different amounts of the one or more 5-HT receptor agonist (e.g., psilocybin), or a pharmaceutically acceptable salt, solvate, metabolite, derivative, or prodrug thereof. In some embodiments, a first amount, a second amount, a third amount, or the like, are the same amounts of the one or more 5-HT receptor agonist (e.g., psilocybin), or a pharmaceutically acceptable salt, solvate, metabolite, derivative, or prodrug thereof. In some embodiments, a first amount, a second amount, a third amount, or the like, are mixed amounts (e.g., two amounts are the same, one is a different amount) of the one or more 5-HT receptor agonist (e.g., psilocybin), or a pharmaceutically acceptable salt, solvate, metabolite, derivative, or prodrug thereof.

In some embodiments, the method further comprises subjecting the individual to one or more stimulus. In some embodiments, the individual is subjected to an initial (e.g., baseline) brain imaging test prior to subjecting the individual the one or more stimulus. In some embodiments, subjecting the individual to one or more stimulus occurs prior to administering the one or more 5-HT receptor agonist (e.g., the first or second amount). In some preferred embodiments, subjecting the individual to one or more stimulus occurs subsequent to administering the one or more 5-HT receptor agonist (e.g., the first or second amount).

In some preferred embodiments, the second brain activity is identified or measured subsequent to (i) subjecting the individual to one or more stimulus and (ii) administering the one or more 5-HT receptor agonist (e.g., the first amount or second amount). In some embodiments, the method comprises administering a therapeutically effective amount of one or more 5-HT receptor agonist (e.g., psilocybin), or a pharmaceutically acceptable salt, solvate, metabolite, derivative, or prodrug thereof, to the individual subsequent to identifying or measuring the second brain activity.

In some embodiments, the second brain activity is a lack of brain activity (e.g., a resting state brain activity). In some embodiments, the resting state brain activity is measured for at least thirty seconds (e.g., across two conditions (e.g., eyes open and eyes closed) (e.g., at least once per week (e.g., after or while the one or more 5-HT agonist is administered to the individual).

In some embodiments, the individual is subjected to the one or more brain imaging test for at least sixty seconds per day. In some embodiments, the individual is subjected to the one or more brain imaging test at least one day per week.

In some embodiments, the one or more stimulus is one or more auditory stimulus.

In some embodiments, the one or more auditory stimulus comprises a first auditory stimulus and a second auditory stimulus (e.g., a paired auditory stimuli). In some embodiments, the first auditory stimulus and the second auditory stimulus are successive.

In some embodiments, the first auditory stimulus and the second auditory stimulus have the same acoustic frequency (e.g., matched paired tones) (e.g., 300 Hertz (Hz) and 300 Hz). In some embodiments, the first auditory stimulus and the second auditory stimulus have a different acoustic frequency (e.g., mismatched paired tones) (e.g., 300 Hz and 700 Hz). In some embodiments, the second auditory stimulus is at least 10 milliseconds (ms) (e.g., 30 ms or more, 50 ms or more, 100 ms or more, 300 ms or more, 500 milliseconds or more, or 1,000 ms or more) after the first auditory stimulus. In some embodiments, the time period between the first auditory stimulus and the second auditory stimulus is fixed. In some embodiments, the time period between the first auditory stimulus and the second auditory stimulus is random.

In some embodiments, the one or more auditory stimulus is one or more sustained auditory stimulus. In some embodiments, the one or more sustained auditory stimulus occurs for at least 500 ms (e.g., 1 second (s)). In some embodiments, the one or more sustained auditory stimulus has a fixed acoustic frequency (e.g., occurring for a period of time of greater than or equal to about five hundred ms). In some embodiments, the one or more sustained auditory stimulus has a variable acoustic frequency (e.g., occurring for a period of time of greater than or equal to about five hundred ms).

In some embodiments, the one or more stimulus is one or more visual stimulus.

In some embodiments, the one or more visual stimulus is an image, a movie, or a contrast (e.g., a shape, a pattern, an emotional face, or the like).

In some embodiments, the one or more visual stimulus occurs for at least 20 ms or more (e.g., a flash). In some embodiments, the one or more visual stimulus occurs for at most ten minutes (e.g., a video).

In some embodiments, the one or more visual stimulus comprises a first visual stimulus and a second visual stimulus (e.g., a paired visual stimuli). In some embodiments, the first visual stimulus and the second visual auditory stimulus are successive. In some embodiments, the second visual stimulus is at least 10 milliseconds (ms) (e.g., 30 ms or more, 50 ms or more, 100 ms or more, 300 ms or more, 500 milliseconds or more, or 1,000 ms or more) after the first visual stimulus. In some embodiments, the time period between the first visual stimulus and the second visual stimulus is fixed. In some embodiments, the time period between the first visual stimulus and the second visual stimulus is random. In some embodiments, the first visual stimulus and the second visual stimulus comprise shapes, patterns, emotional faces, and/or colors, and have the same contrast. In some embodiments, the first visual stimulus and the second visual stimulus comprise shapes, patterns, emotional faces, and/or colors, and have a different contrast.

In some embodiments, the second amount of one or more 5-HT receptor agonist (e.g., psilocybin), or the pharmaceutically acceptable salt, solvate, metabolite, derivative, or prodrug thereof, is administered to the individual subsequent to identifying or measuring a significant change in brain activity. In some embodiments, the significant change in brain activity is from a baseline brain activity measurement to the first brain activity measurement. In some embodiments, the significant change in brain activity is from the first brain activity measurement and the second brain activity measurement. In some embodiments, the significant change in brain activity is identified or measured between two brain activity measurements identified or measured between the second brain activity measurement and another brain activity measurement. In some embodiments, the significant change in brain activity is identified or measured subsequent to the second brain activity measurement.

In some embodiments, a change in brain activity between the first brain activity and the second brain activity (e.g., in a particular region of the brain (e.g., a region of the prefrontal cortex)) indicates that the therapeutically effective dose of the one or more 5-HT agonist has been administered to the individual. In some embodiments, an increase in brain activity between the first brain activity and the second brain activity (e.g., in a particular region of the brain (e.g., a region of the prefrontal cortex)) indicates that the therapeutically effective dose of the one or more 5-HT agonist has been administered to the individual. In some embodiments, a decrease in brain activity between the first brain activity and the second brain activity indicates that the therapeutically effective dose of the one or more 5-HT agonist has been administered to the individual.

In some embodiments, a change in the resting state brain activity (e.g., of the prefrontal cortex of the individual) indicates the therapeutically effective dose of the one or more 5-HT agonist has been administered to the individual.

In some embodiments, the brain activity or the lack thereof is measured in a time domain (e.g., as time-locked changes) to a paired auditory stimuli as a voltage (e.g., evoked potentials), a magnetic field, or a hemodynamic signal (e.g., flux, flow, velocity, or oxygenation level).

In some embodiments, the brain activity or the lack thereof is measured in a frequency domain as a voltage (e.g., power in alpha bands, beta bands, gamma bands, delta bands, or theta bands), a magnetic field, or a hemodynamic signal (e.g., flux, flow, velocity, or oxygenation level).

In some embodiments, the second amount is the same dose as the first amount. In some embodiments, the second amount is a lower dose than the first amount.

In some embodiments, the first amount of the one or more 5-HT receptor agonist (e.g., psilocybin) is sufficient to elicit a measurable change in brain activity. In some embodiments, the first amount of the one or more 5-HT receptor agonist (e.g., psilocybin) is sufficient to elicit a significant change in brain activity.

In some embodiments, the first amount of the one or more 5-HT receptor agonist (e.g., psilocybin) does not elicit a measurable change in brain activity. In some embodiments, the first amount of the one or more 5-HT receptor agonist (e.g., psilocybin) does not elicit a significant change in brain activity.

In some embodiments, a lack of a significant change in brain activity is identified or measured. In some embodiments, the lack of a significant change in brain activity is identified or measured subsequent to the first brain activity measurement. In some embodiments, the lack of a significant change in brain activity is identified or measured subsequent to the second brain activity measurement. In some embodiments, the lack of a significant change in brain activity is identified or measured from the first brain activity measurement and the second brain activity measurement.

In some embodiments, the second amount of one or more 5-HT receptor agonist (e.g., psilocybin), or the pharmaceutically acceptable salt, solvate, metabolite, derivative, or prodrug thereof, is administered to the individual subsequent to identifying or measuring a lack of significant change in brain activity. In some embodiments, the second amount is a lower dose than the first amount. In some embodiments, the second amount is a higher dose than the first amount. In some embodiments, a lack of a significant change in brain activity is identified or measured and the second amount is a higher dose than the first amount.

In some embodiments, the second amount of the one or more 5-HT receptor agonist (e.g., psilocybin) is sufficient to elicit a measurable change in brain activity. In some embodiments, the second amount of the one or more 5-HT receptor agonist (e.g., psilocybin) is sufficient to maintain a measurable change in brain activity. In some embodiments, the second amount of the one or more 5-HT receptor agonist (e.g., psilocybin) is sufficient to elicit a significant change in brain activity. In some embodiments, the second amount of the one or more 5-HT receptor agonist (e.g., psilocybin) is sufficient to maintain a significant change in brain activity.

In some embodiments, the second amount of the one or more 5-HT receptor agonist (e.g., psilocybin) does not elicit a measurable change in brain activity. In some embodiments, the second amount of the one or more 5-HT receptor agonist (e.g., psilocybin) does not elicit a significant change in brain activity.

In some embodiments, the measurable change is from the first brain activity measurement and another brain activity measurement. In some embodiments, the measurable change is from the first brain activity measurement and the second brain activity measurement. In some embodiments, the measurable change is from the first brain activity measurement and a third brain activity measurement, a fourth brain activity measurement, a fifth brain activity measurement, or the like. In some embodiments, the measurable change is from the second brain activity measurement and a third brain activity measurement, a fourth brain activity measurement, a fifth brain activity measurement, or the like.

In some embodiments, the method further comprises identifying or measuring another brain activity by performing a third (e.g., fourth, fifth, six, or more) brain evaluation (e.g., a third (e.g., fourth, fifth, six, or more) electroencephalogram (EEG)) on the individual. In some embodiments, the third or more brain evaluation is performed on the individual subsequent to administering the second amount (e.g., a third amount, fourth amount, fifth amount, or more) of the one or more 5-HT receptor agonist (e.g., psilocybin), or a pharmaceutically acceptable salt, solvate, metabolite, derivative, or prodrug thereof.

In some embodiments, the first amount of the one or more 5-HT receptor agonist (e.g., psilocybin) is the same as the second amount of the one or more 5-HT receptor agonist (e.g., psilocybin).

In some embodiments, the first amount of the one or more 5-HT receptor agonist (e.g., psilocybin) is different than the second amount of the one or more 5-HT receptor agonist (e.g., psilocybin).

In some embodiments, a 5HT receptor agonist is administered to an individual at a first time point and a second time point. In some embodiments, the first time and the second time are within a 24 hour period (e.g., within a 4 hour period, within a 6 hour period, within an 8 hour period, or the like). In some embodiments, the first time and the second time are on or after a 24 hour period (e.g., the first time is more than or equal to 24 hours after the second time, the first time is more than or equal to 48 hours after the second time, or the like). In certain instances, a first dose comprising a 5HT receptor agonist and a second dose of a 5HT receptor agonist are administered to an individual. In some embodiments, the first dose and the second dose are different. In some embodiments, the first dose and the second dose are the same. In some embodiments, additional doses are also administered, such as on intervening days and/or subsequent to administration of the second dose.

In some embodiments, the second amount of the one or more 5-HT receptor agonist (e.g., psilocybin) is administered at least one hour (e.g., one hour or more, two hours or more, three hours or more, four hours or more, or the like) after the first amount of the one or more 5-HT receptor agonist (e.g., psilocybin).

In some embodiments, the first amount of the one or more 5-HT receptor agonist (e.g., psilocybin) and the second amount of the one or more 5-HT receptor agonist (e.g., psilocybin) are administered within a 24 hour period (e.g., within a 4 hour period, within a 6 hour period, within an 8 hour period, or the like) of each other.

In some embodiments, the second amount is administered more than or equal to 24 hours after the first amount is administered to the individual (e.g., the second amount is administered more than or equal to 36 hours after the first amount is administered, the second amount is administered more than or equal to 48 hours after the first time is administered, or the like).

In some embodiments, a third amount (or more (e.g., a fourth amount, a fifth amount, or more)) of one or more 5-HT receptor agonist (e.g., psilocybin) is administered to the individual (e.g., between the first and second amount (e.g., as in intervening dose) or after the second amount (e.g., as a subsequent dose)). In some embodiments, the third amount (or more) is a therapeutically effective amount of the one or more 5-HT receptor agonist (e.g., psilocybin). In some embodiments, the third amount or more is administered between the first and second amount (e.g., as in intervening dose). In some embodiments, the third amount or more is administered after the second amount (e.g., as a subsequent dose).

In some embodiments, the therapeutically effective amount of the one or more 5-HT receptor agonist, or a pharmaceutically acceptable salt, solvate, metabolite, derivative, or prodrug thereof, is administered to the individual in need thereof over an extended period of time. In some embodiments, the therapeutically effective amount of the one or more 5-HT receptor agonist, or a pharmaceutically acceptable salt, solvate, metabolite, derivative, or prodrug thereof, is administered to the individual over an extended period of time such that the level of active 5-HT receptor agonist does not exceed 10 ng/mL in the individual (e.g., for the entirety of the extended period of time). In some embodiments, the therapeutically effective amount of the one or more 5-HT receptor agonist, or a pharmaceutically acceptable salt, solvate, metabolite, derivative, or prodrug thereof, is administered to the individual in need thereof over an extended period of time such that the level of active 5-HT receptor agonist does not fall below 0.01 ng/mL in the individual (e.g., for the entirety of the extended period of time). In some embodiments, the therapeutically effective amount of the one or more 5-HT receptor agonist, or a pharmaceutically acceptable salt, solvate, metabolite, derivative, or prodrug thereof, is administered to the individual in need thereof over an extended period of time such that the level of active 5-HT receptor agonist does not exceed 10 ng/mL and does not fall below 0.01 ng/mL in the individual (e.g., for the entirety of the extended period of time).

In some embodiments, the extended period of time is for an entire treatment plan tailored for the individual in need thereof. In some embodiments, the extended period of time is one day, one week, two weeks, one month, six months, one year, or more. In some embodiments, the individual in need thereof is administered the one or more 5-HT receptor agonist, or the pharmaceutically acceptable salt, solvate, metabolite, derivative, or prodrug thereof, for at least one week. In some embodiments, the individual in need thereof is administered the one or more 5-HT receptor agonist, or the pharmaceutically acceptable salt, solvate, metabolite, derivative, or prodrug thereof, daily for a week or more, twice per day for a week or more, three times per day for a week or more, every other day for a week or more, two times a week, once a week, bi-weekly for a month or more, or the like.

In some instances, the formulation is any formulation provided herein. In some instances, the formulation is an oral formulation, a dermal formulation, a buccal formulation, a nasal formulation, an intraparietal (IP) formulation, or an inhalation formulation. In some embodiments, the oral formulation is in a solid form or a liquid form.

In some embodiments, the formulation releases the active 5-HT agonist in the individual in need thereof for a period of at least two hours. In some embodiments, the formulation releases the active 5-HT agonist in the individual in need thereof for a period of at most two hours. In some embodiments, the formulation releases the active 5-HT agonist in the individual in need thereof for a period of at least one day. In some embodiments, the formulation releases the active 5-HT agonist in the individual in need thereof for a period of at most one day. In some embodiments, the formulation releases the active 5-HT agonist in the individual in need thereof for a period of two hours to one week.

In various embodiments provided herein, the (e.g., pharmaceutical) composition, formulation, or dosage of the (e.g., one or more) 5-HT receptor agonist is any suitable 5-HT receptor agonist, such as a 5-HT2 receptor agonist, including free bases thereof, salts thereof, prodrugs thereof, metabolites thereof, or the like. In specific embodiments, the one or more 5-HT receptor agonist is psilocybin or psilocin, or a pharmaceutically acceptable salt, solvate, metabolite, derivative, or prodrug thereof.

In some embodiments, the one or more 5-HT receptor agonist, or a pharmaceutically acceptable salt, solvate, metabolite, derivative, or prodrug thereof, is administered to the individual in an amount of about 0.01 mg to about 100 mg (e.g., about 1 mg to about 20 mg, about 1 mg to about 5 mg, about 5 mg to about 10 mg, or the like). In some embodiments, the one or more 5-HT receptor agonist, or a pharmaceutically acceptable salt, solvate, metabolite, derivative, or prodrug thereof, is administered to the individual in an amount of about 0.1 mg to about 25 mg.

In some embodiments, the one or more evaluations (e.g. brain test) is performed using a wearable device (e.g., brain imaging). In some embodiments, the one or more evaluations or the lack thereof is recorded to a local memory or over a network using a mobile device (e.g., through a mobile application). In some embodiments, the method comprises integrating electronic patient reported outcomes or subjective mood, affect, coping, sleep quality, stress, anxiety, memory, and other emotional or functional data with brain imaging or brain activity levels. In some embodiments, the method further comprises providing data (e.g., patterns, clusters, classifications, amplitudes, frequencies, or magnitude) on a device (e.g., a phone, tablet, or computer) to the individual and/or a caregiver (e.g., physicians, counselors, psychologist, or spiritual leader), the data comprising visual representations of brain responses or brain activity data and/or survey or patient reported outcomes to help convey the effectiveness or lack thereof of a psychedelic therapy.

In some embodiments, the effect on the individual (e.g., from an active form of a 5-HT agonist in the individual) is measured by obtaining a measurement of a physiological response or reaction (e.g., electroencephalogram (EEG), electrocardiogram (ECG or EKG), pulse rate, oximetry, or the like) of the individual (e.g., prior or subsequent to the administration of the one or more 5-HT receptor agonist).

In some embodiments, the method further comprises performing one or more heart test (e.g., to determine heart rate or cardiovascular activity) on the individual (e.g., for at least 30 seconds) (e.g., the one or more heart test being measured using a separate electrode sensitive voltage or sensor (e.g., photodiode) sensing an optical measure from visible light or infrared light).

In some embodiments, the method further comprises performing one or more test to measure skin conductance or impedance.

In some embodiments, the effect on the individual (e.g., from an active form of a 5-HT agonist in the individual) is measured by obtaining a biological sample from the individual. In some embodiments, the biological sample is serum, plasma, whole blood, urine, or the like.

In some embodiments, the one or more 5-HT receptor agonist, or a pharmaceutically acceptable salt, solvate, metabolite, derivative, or prodrug thereof is a 5HT2 receptor agonist. In some embodiments, the one or more 5-HT receptor agonist, or a pharmaceutically acceptable salt, solvate, metabolite, derivative, or prodrug thereof is a hallucinogenic compound. In some embodiments, the hallucinogenic compound is psilocybin or and psilocin, or a pharmaceutically acceptable salt, solvate, metabolite, derivative, or prodrug thereof.

In some embodiments, the hallucinogenic compound produces a hallucinogenic effect in the individual. In some embodiments, the hallucinogenic compound, or a pharmaceutically acceptable salt, solvate, metabolite, derivative, or prodrug thereof, produces an adverse event or a clinically important effect in the individual. In some embodiments, the clinically important effect is a clinically important impairment of the individual, altered perception, altered cognition, impaired attention, drowsiness, and/or confusion. In some embodiments, the altered perception in the individual is a visual perception alteration, an auditory perception alteration, bodily perception alteration, a temporal perception alteration, or a spatial perception alteration. In some embodiments, the hallucinogenic compound is (e.g., frequently) administered at a dose that does not produce a hallucinogenic effect in the individual. In some embodiments, the hallucinogenic compound is (e.g., frequently) administered at a dose that does not produce an adverse event in the individual.

In some embodiments, the one or more 5-HT receptor agonist, or a pharmaceutically acceptable salt, solvate, metabolite, derivative, or prodrug thereof is psilocybin or and psilocin, or a pharmaceutically acceptable salt, solvate, metabolite, derivative, or prodrug thereof. In some embodiments, the one or more 5-HT receptor agonist, or a pharmaceutically acceptable salt, solvate, metabolite, derivative, or prodrug thereof is psilocybin. In some embodiments, the active form of the 5-HT receptor agonist (e.g., in the individual) is psilocin.

In some embodiments, the one or more 5-HT receptor agonist, or a pharmaceutically acceptable salt, solvate, metabolite, derivative, or prodrug thereof, is administered to the individual in an amount and/or formulation to provide a plasma concentration of an active 5-HT receptor agonist within 2 hours or less (1.5 hours or less, 1 hour or less, 30 minutes or less, or the like).

In some embodiments, the one or more 5-HT receptor agonist, or a pharmaceutically acceptable salt, solvate, metabolite, derivative, or prodrug thereof, is administered to the individual in an amount and/or formulation to provide a plasma concentration of the active 5-HT receptor agonist of at least 0.01 ng/mL (e.g., 0.01 ng/mL or more, 0.1 ng/mL or more, 1 ng/mL or more, 10 ng/mL or more, or the like) in the individual (e.g., for at least 3 hours (e.g., 3 hours or more, 6 hours or more, 8 hours or more, 12 hours or more, 18 hours or more, 24 hours or more, or the like)).

In some embodiments, the level of active 5-HT receptor agonist is maintained in the individual at a concentration of more than or equal to 0.01 ng/mL (e.g., more than 0.01 ng/mL, more than 0.05 ng/mL, more than 0.1 ng/mL, more than 0.5 ng/mL, more than 1 ng/mL, more than 5 ng/mL, more than 10 ng/mL, or more than 10 ng/mL). In some embodiments, the level of active 5-HT receptor agonist is maintained in the individual at a concentration less than or equal to 20 ng/mL (e.g., less than 20 ng/mL, less than 10 ng/mL, less than 5 ng/mL, less than 1 ng/mL, less than 0.5 ng/mL, less than 0.1 ng/mL, less than 0.05 ng/mL, or less than 0.01 ng/mL). In some embodiments, the level of the active 5-HT receptor agonist is maintained in the individual from about 0.01 ng/mL to about 10 ng/mL. In some embodiments, the level of the active 5-HT receptor agonist in the individual is from about 0.01 ng/mL to about 10 ng/mL.

In some embodiments, the composition, formulation, or dosage form is a pharmaceutical composition. In specific embodiments the pharmaceutical composition is a dosage form, such as a discrete dosage form. In more specific embodiments, the pharmaceutical composition is a discrete oral dosage form.

In some embodiments, the therapeutically effective amount of 5-HT receptor agonist is an amount insufficient to provide a hallucinogenic experience (or other adverse effect) (e.g., in an average adult).

In some embodiments, the pharmaceutical composition is a low-dose pharmaceutical composition. In specific embodiments, the low-dose pharmaceutical composition is a pharmaceutical composition (e.g., dosage form) such that following administration to an individual in need thereof, the low-dose pharmaceutical composition provides a maximum plasma concentration (Cₘₐₓ) of the 5-HT receptor agonist (e.g., or active metabolite(s) thereof) of less than 6 ng/mL in the individual in need thereof. In more specific embodiments, following administration to an individual in need thereof, the low-dose pharmaceutical composition provides a maximum plasma concentration (Cₘₐₓ) of the 5-HT receptor agonist (e.g., or active metabolite(s) thereof) of at least 0.5 ng/mL and less than 6 ng/mL in the individual in need thereof (e.g., about 1 ng/mL to about 5.5 ng/mL, about 2 ng/mL to about 5 ng/mL, or the like).

In some embodiments, the one or more 5-HT receptor agonist, or a pharmaceutically acceptable salt, solvate, metabolite, derivative, or prodrug thereof, is administered to the individual in an amount and/or formulation to provide a maximum plasma concentration (Cₘₐₓ) of the active 5-HT receptor agonist of about 20 ng/mL or less in the individual. In some embodiments, the level of the active 5-HT receptor agonist in the individual (e.g., in a biological sample (e.g., serum, plasma, or whole blood) of the individual) is about 0.01 ng/mL to about 20 ng/mL. In some embodiments, the level of the active 5-HT receptor agonist in the individual (e.g., in a biological sample (e.g., serum, plasma, or whole blood) of the individual) is about 1 ng/mL to about 15 ng/mL.

In various embodiments, the pharmaceutical composition is formulated in any suitable manner. In some embodiments, the one or more 5-HT receptor agonist, or a pharmaceutically acceptable salt, solvate, metabolite, derivative, or prodrug thereof, is administered to the individual in need thereof as an oral formulation, an intravenous formulation, a dermal formulation, a buccal formulation, a nasal formulation, or an inhalation formulation.

In certain preferred embodiments, the pharmaceutical composition or dosage form is formulated for oral administration. Other formulations are also contemplated herein, however, including intravenous formulations, buccal formulations, nasal formulations, inhalation formulations, or any other suitable formulation.

In various embodiments herein, a composition (e.g., pharmaceutical composition, dosage form, combination or formulation) provided herein is administered at any frequency. For example, in some embodiments, a single dose is provided. In other embodiments, the composition is or is formulated to be administered to a subject in need thereof at least once a day. In other embodiments, the composition is or is formulated to be administered to a subject in need thereof once a day. In other embodiments, the composition is or is formulated to be administered to a subject in need thereof at least twice a day. In various other embodiments, the composition is or is formulated for twice daily, once daily, twice weekly, thrice weekly, or the like administration.

In various embodiments, such a composition is formulated to have any of the components and/or features as described for a composition provided herein, such as described above. In some embodiments, such a composition comprises one or more pharmaceutically acceptable excipient.

In specific embodiments, such a method is provided for of treating or managing a neurological condition or the symptoms thereof in a subject in need thereof (e.g., in a subject suffering from or susceptible to the neurological condition).

In various embodiments provided herein, the (e.g., one or more) 5-HT receptor agonist is any suitable 5-HT receptor agonist, such as a 5-HT2 receptor agonist, including free bases thereof, salts thereof, prodrugs thereof, metabolites thereof, and the like. In various embodiments provided herein, the (e.g., one or more) 5-HT receptor agonist is any suitable 5-HT receptor agonist, such as a 5-HT2A receptor agonist, including free bases thereof, salts thereof, prodrugs thereof, metabolites thereof, and the like. In specific embodiments, the (e.g., one or more) 5-HT receptor agonist is psilocybin or psilocin, or a pharmaceutically acceptable salt, solvate, metabolite, derivative, or prodrug thereof.

In certain preferred embodiments, the method comprises oral administration. Other routes of administration are also contemplated herein, however, including buccal administration, nasal administration, inhalation administration, or any other suitable administration routes.

In some embodiments, the method is for treating a mental, behavioral, or neuropsychiatric condition, or the symptoms thereof. In some embodiments, the method is for managing a mental, a behavioral, or a neuropsychiatric condition, or the symptoms thereof. In some embodiments, the method is for treating and managing a mental, behavioral, or neuropsychiatric condition, or the symptoms thereof. In some embodiments, the individual is suffering from or susceptible to the mental, a behavioral, or a neuropsychiatric condition. In certain instances, the symptoms of the mental, a behavioral, or a neuropsychiatric condition are physical, behavioral, emotional, mental, or a combination thereof.

In some embodiments, the mental, the behavioral, or the neuropsychiatric condition is any disease or disorder provided herein. In some embodiments, the mental, the behavioral, or the neuropsychiatric condition is a symptom (e.g., provided herein) of any disease or disorder provided herein. In some embodiments, the mental, the behavioral, or the neuropsychiatric condition is a chronic condition.

In some embodiments, the mental, the behavioral, or the neuropsychiatric condition is a Diagnostic and Statistical Manual of Mental Disorders (DSM-5) category disease or disorder). In some embodiments, the mental, the behavioral, or the neuropsychiatric condition is a non-DSM-5 category disease or disorder.

In some embodiments, the mental, the behavioral, or the neuropsychiatric condition is an attention (e.g., an attention deficit) or a cognitive (e.g., neurocognitive) disorder or condition. In some embodiments, the mental, the behavioral, or the neuropsychiatric condition is a neurocognitive disorder or condition. In some embodiments, the mental, the behavioral, or the neuropsychiatric condition is an attention deficit disorder or condition. In some embodiments, the cognitive condition is mild cognitive impairment, dementia, or Alzheimer's disease.

In some embodiments, the mental, the behavioral, or the neuropsychiatric condition is induced by stress and/or anxiety.

In some embodiments, the mental, the behavioral, or the neuropsychiatric condition is selected from the group consisting of addiction, anxiety, apathy, attention (e.g., the lack thereof), and depression (e.g., moderate depression). In some embodiments, the mental, the behavioral, or the neuropsychiatric condition is PTSD, constructive impulsivity, a phobia, a fear, or the like. In some embodiments, the mental, the behavioral, or the neuropsychiatric condition is major depressive disorder.

In some embodiments, the individual is susceptible to or suffering from a brain disorder or condition (e.g., a mental condition, a behavioral condition, a neuropsychiatric condition, a brain state or a lack thereof (e.g., coping, motivation, stress, depression or anxiety), encephalitis, or a brain dysfunction (e.g., cognitive decline or brain fog).

In some embodiments, the condition is attention deficit hyperactivity disorder (ADHD) or attention deficit disorder (ADD).

In some embodiments, the condition is mild cognitive impairment, dementia, or Alzheimer's disease.

In some embodiments, the condition is addiction.

In various embodiments, methods provided herein are suitable for treating any suitable disorder, such as a neurological condition, such as a neurological disorder, or symptoms thereof. In specific embodiments, the neurological condition is a neurocognitive disorder. In various embodiments, methods provided herein are suitable for treating any suitable disorder or symptoms thereof including, but not limited to, feelings of distress, futility, disempowerment, despair, helplessness, hopelessness, and the like. In some embodiments, the disorder causing the feelings of distress, futility, disempowerment, despair, helplessness, hopelessness, and the like is a mood disorder. In some embodiments, the disorder causing the feelings of distress, futility, disempowerment, despair, helplessness, hopelessness and the like is a mood disorder, that clinically presents as demoralization. In some embodiments, symptoms of the neurological condition are physical, behavioral, emotional, mental or a combination thereof. In some embodiments, the neurological condition is an addictive disorder (e.g., alcohol abuse, substance abuse, smoking, or obesity). In some embodiments, the neurological condition is an eating disorder or an auditory disorder. In some embodiments, the neurological condition is pain (e.g., chronic pain). In some embodiments, the neurological condition is depression, bipolar disorder, post-traumatic stress disorder (PTSD), panic disorder, phobia, schizophrenia, psychopathy, or antisocial personality disorder. In some embodiments, the neurological condition is an impulsive disorder. In some embodiments, the impulsive disorder is attention deficit hyperactivity disorder (ADHD), Tourette's syndrome or autism. In some embodiments, the neurological condition is a compulsive disorder (e.g., obsessive compulsive disorder (OCD), gambling, or aberrant sexual behavior). In some embodiments, the neurological condition is a personality disorder (e.g., conduct disorder, antisocial personality, or aggressive behavior).

In some embodiments, administration to a subject in need thereof occurs several times per day. In some embodiments, administration to a subject in need thereof occurs no more frequently than once a day (e.g., no more frequently than once every other day, no more frequently than once every third day, no more frequently than twice a week, no more frequently than once a week, no more frequently than once every two weeks, or the like). In some embodiments, administration to a subject in need thereof occurs once a day, every alternate day, three times a week, twice a week, once a week, every other week, two weeks per month, three weeks per month, once a month, twice a month or three times per month. In specific embodiments, administration is about once a day. In other specific embodiments, administration is about every alternate day. In still other specific embodiments, administration is about once a week.

In various embodiments, administration continues for any suitable length of time, such as at least one day, at least one week, at least 1 month, 2 months, 3 months, 4 months, 5 months, 6 months, 7 months, 8 months, 9 months, 10 months, 11 months, 12 months, 18 months, 2 years, 3 years, or more.

Provided in some embodiments here is a method for monitoring the effectiveness of a 5-hydroxytryptamine (5-HT) receptor agonist (e.g., psilocybin), comprising measuring (e.g., on a network (e.g., WiFi, cloud, BLE, 3/4/5G)) one or more brain response using a node or a wearable brain imaging device (e.g., an EEG), wherein the patient listens to paired or sustained auditory stimuli (e.g., at home, at work, or at school), the auditory stimuli being delivered from an application on a (e.g., mobile) device (e.g., cellular phone, tablet, or the like).

Provided in some embodiments herein is a method for using Bayesian statistics and methods to estimate and predict confidence in the effectiveness of a psychedelic treatment or dose based on historical brain imaging data in a database for particular patient types.

Provided in some embodiments herein is a method of imaging brain activity (e.g., voltage) in an individual using a wearable device (e.g., EEG (e.g., multi-electrode EEG device)), the wearable device having two or more electrodes that make contact with the forehead of the individual for at least thirty seconds and up to twenty minutes.

Provided in some embodiments herein is a method for optically imaging brain activity in an individual (e.g., from a wearable device) to determine, track, or optimize the effectiveness of a psychedelic treatment, wherein a brain signal is identified by transmission of visible light (400-680 nanometers) or infrared light (greater than or equal to 680 nanometers) across the skin into the brain to collect reflected photons by a sensor (e.g., a photodiode or CMOS).

Provided in some embodiments is a method of acoustically imaging brain activity in an individual (e.g., from a wearable device) to determine, track, or optimize the effectiveness of a psychedelic treatment, wherein a brain signal is identified by transmission of ultrasound (e.g., having an acoustic frequency of greater than or equal to one megahertz) (e.g., across the skin and skull) into the brain to collect reflected ultrasound waves by a sensor (e.g., a piezoelectric resonant material (e.g., a PZT, a CMUT, a PMUT).

In some embodiments, the method comprises using artificial intelligence (e.g., deep convolution neural networks) algorithms and/or machine learning processes to classify, cluster, or recognize patterns or relationships amongst sensory-evoked (e.g., auditory or visual stimuli) brain activity or resting state brain activity acquired by brain imaging methods and patient reported outcomes (e.g., changes in mood, anxiety, motivation, or memory) for optimizing the dosing schedule or treatment paradigm or a patient undergoing treatment with psychedelic substances (e.g. psilocybin).

Provided in some embodiments herein is a computer-implemented method for identifying an effectiveness of a psychedelic treatment administered to an individual.

Provided in some embodiments herein is a computer-implemented method for identifying a therapeutically effective dose of a 5-HT receptor agonist administered to an individual. In some embodiments, the method comprises administering the 5-HT receptor agonist to the individual. In some embodiments, the method comprises emitting one or more (e.g., auditory or visual) to the individual. In some embodiments, the method comprises receiving, from a brain imaging device (e.g., an EEG), a brain response. In some embodiments, the method comprises identifying the therapeutically effective dose of the 5-HT receptor agonist based at least in part on the one or more (e.g., auditory or visual) stimulus and the brain response.

In some embodiments, the method further comprises receiving, from the individual, an emotional data. In some embodiments, the therapeutically effective dose of the 5-HT receptor agonist is further determined based on the emotional data. In some embodiments, the emotional data comprises a mood rating, a sleep rating, a stress rating, an anxiety rating, a memory rating, or any combination thereof.

In some embodiments, the method comprises transmitting (e.g., the effectiveness of) the therapeutically effective dose of the 5-HT receptor agonist to the individual, a caregiver, or both.

In some embodiments, two or more of any step provided herein are performed simultaneously.

In some embodiments, two or more of any step provided herein are performed sequentially.

In some embodiments, (e.g., the effectiveness of) the therapeutically effective dose of the 5-HT receptor agonist is transmitted by a mobile device (e.g., cellular phone, tablet). In some embodiments, (e.g., the effectiveness of) the therapeutically effective dose of the 5-HT receptor agonist is transmitted over a wireless network (e.g., WiFi, cloud, BLE, 3/4/5G).

In some embodiments, (e.g., the effectiveness of) the therapeutically effective dose of the 5-HT receptor agonist is determined by a machine learning algorithm.

Provided in some embodiments herein is a computer-implemented system for identifying an effectiveness of a psychedelic treatment administered to an individual.

Provided in some embodiments herein is a computer-implemented system for identifying a therapeutically effective dose of a 5-HT receptor agonist administered to an individual.

In some embodiments, the system comprises a digital processing device. In some embodiments, the digital processing device comprises at least one processor. In some embodiments, the digital processing device comprises an operating system configured to perform executable instructions. In some embodiments, the digital processing device comprises a memory. In some embodiments, the digital processing device comprises a computer program including instructions executable by the digital processing device to create an application.

Provided in some embodiment herein is a non-transitory computer-readable storage media encoded with a computer program including instructions executable by a processor to create an application for identifying an effectiveness of a psychedelic treatment administered to an individual.

Provided in some embodiment herein is a non-transitory computer-readable storage media encoded with a computer program including instructions executable by a processor to create an application for identifying a therapeutically effective dose of a 5-HT receptor agonist administered to an individual.

In some embodiments, the application is configured to receive an indicator of the 5-HT receptor agonist administered to the 5-HT receptor agonist provided to the individual. In some embodiments, the application is configured to direct a device (e.g., an auditory or visual device) to emit one or more (auditory or visual) stimulus to the individual. In some embodiments, the application is configured to receive, from a brain imaging device (e.g., EEG), a brain response. In some embodiments, the application is configured to identify the therapeutically effective dose of the 5-HT receptor agonist based at least in part on the one or more (auditory or visual) stimulus and the brain response.

In some embodiments, the application is further configured to receive, from the individual, an emotional data. In some embodiments, the application is further configured to determine the therapeutically effective dose of the 5-HT receptor agonist based on the emotional data.

In some embodiments, the emotional data comprises a mood rating, a sleep rating, a stress rating, an anxiety rating, a memory rating, or any combination thereof.

In some embodiments, the application is configured to simultaneously perform two or more of steps provided herein.

In some embodiments, the application is configured to sequentially perform two or more of steps provided herein.

In some embodiments, the application is further configured to transmit the therapeutically effective dose of the 5-HT receptor agonist to the individual, a caregiver, or both. In some embodiments, the application directs the transmission of the therapeutically effective dose of the 5-HT receptor agonist by a mobile device (e.g., cellular phone, tablet). In some embodiments, the application directs the transmission of the therapeutically effective dose of the 5-HT receptor agonist over a wireless network (e.g., WiFi, cloud, BLE, 3/4/5G). In some embodiments, the therapeutically effective dose of the 5-HT receptor agonist is determined by a machine learning algorithm.

Provided in some embodiments herein is a computer-implemented system for identifying an effectiveness of a psychedelic treatment administered to an individual.

Provided in some embodiments herein is a computer-implemented system for identifying a therapeutically effective dose of a 5-HT receptor agonist administered to an individual. in some embodiments, the system comprises a digital processing device. In some embodiments, the digital processing device comprises at least one processor. In some embodiments, the digital processing device comprises an operating system configured to perform executable instructions. In some embodiments, the digital processing device comprises a memory. In some embodiments, the digital processing device comprises a computer program including instructions executable by the digital processing device to create an application.

Provided in some embodiments is a non-transitory computer-readable storage media encoded with a computer program including instructions executable by a processor to create an application for identifying an effectiveness of a psychedelic treatment administered to an individual.

Provided in some embodiments is a non-transitory computer-readable storage media encoded with a computer program including instructions executable by a processor to create an application for identifying a therapeutically effective dose of a 5-HT receptor agonist administered to an individual.

In some embodiments, the application is configured to apply a first machine learning algorithm to a plurality of 5-HT receptor agonist doses (e.g., dose responses), a plurality of (e.g., auditory or visual) stimuli, and brain responses to identifying the therapeutically effective dose of the 5-HT receptor agonist.

In some embodiments, the application is configured to receive verified emotional data regarding an emotional effectiveness of the 5-HT receptor agonist.

In some embodiments, the application is configured to feed back the verified data to improve the first machine learning algorithm's calculation over time.

In some embodiments, the first machine learning algorithm is trained by a neural network. In some embodiments, the neural network comprises a first training module for creating a first training set comprising a set of 5-HT receptor agonist doses (e.g., dose responses), each dose associated with one (e.g., auditory or visual) stimulus, and one brain response. In some embodiments, the neural network comprises a first training module training the neural network using the first training set. In some embodiments, the neural network comprises a second training module creating a second training set for second stage training comprising the first training set and the psychedelic treatments incorrectly detected as having a positive effectiveness on the individual after the first stage of training. In some embodiments, the neural network comprises training the neural network using the second training set.

In some embodiments, the application is configured to perform administering (e.g., at least once weekly for 5 or more weeks) one or more questionnaires to the individual to acquire emotional data. In some embodiments, questionnaires are administered once, twice, three times, four times, five times, six times, seven times, eight times, nine times, ten times, eleven times, twelve times, thirteen times, or fourteen times of more weekly for at least 1, 2, 3, 4, 5, 6, 7, 8, 9, 10, 11, or 12 or more weeks, or for 4, 5, 6, 7, 8, 9, 10, 11, 12 or more months, or for 1, 2, 3, or 4 or more years.

In some embodiments, the emotional data comprises data regarding depression, anxiety, stress, coping, mood, sleep, and quality of life of the individual.

In some embodiments, the one or more questionnaires can include a Beck Depression Inventory (BDI), a Generalized Anxiety Disorder (GAD-7), a Depression Anxiety Stress Scale (DASS), a Brief-COPE, a Positive and Negative Affect Schedule (PANAS), a State Trait Anxiety Inventory (STAI), a modified version of a Russel Mood Circumplex, a modified version of a NIH Sleep Diary, a 36 Item Short Form Health Survey (SF-36), a 5D Altered state of Consciousness Scale (5d-ASC), and/or a daily sleep diary questionnaire.

In some embodiments, the application is configured to perform receiving (e.g., at least once weekly for 5 or more weeks) an individual's responses to the one or more questionnaires.

In some embodiments, the application is configured to perform scoring the individual's responses to the one or more questionnaires.

In some embodiments, identifying the therapeutically effective dose of the 5-HT receptor agonist is based at least in part on the scoring of the one or more questionnaires.

In some embodiments, the application is configured to perform receiving resting state brain activity from the individual. In some embodiments, the resting state brain activity is measured for at least thirty seconds (e.g., at least once, and up to four times, daily). In some embodiments, identifying the therapeutically effective dose of the 5-HT receptor agonist is based at least in part on changes to the resting state brain activity.

In some embodiments, receiving, from a brain imaging device (e.g., EEG), a brain response, comprises receiving amplitude and spectral power of EEG potentials measured from frontal, temporal, and parietal EEG sites in response to the one or more auditory and/or visual stimulus. In some embodiments, the one or more auditory stimulus comprises one or more auditory tasks comprising P50 paired click auditory suppression, Mismatch Negativity (MMN), and/or Auditory Steady State Response (ASSR). In some embodiments, the one of more auditory tasks is administered to the individual 1.5 hours after the 5-HT receptor agonist is administered to the individual. In some embodiments, the one or more visual stimulus comprises one or more visual tasks comprising an Emotional Flanker Task and/or a Continuous Performance Test (CPT). In some embodiments, identifying the therapeutically effective dose of the 5-HT receptor agonist is based at least in part on the brain response received to the one or more auditory tasks and/or the one or more visual tasks.

In some embodiments, identifying the therapeutically effective dose of the 5-HT receptor agonist is performed using one or more statistical methods comprising Bayesian methods, Mixed Model Repeated Measures (MMRM), repeated measures ANOVA and ANCOVA, and regression analyses.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows an ascending dose protocol for titrating a therapeutically effective dose of a 5-hydroxytryptamine (5-HT) agonist provided herein.

FIG. 2 shows a Mismatch Negativity (MMN)/Auditory Steady State Response (ASSR) protocol for titrating a therapeutically effective dose of a 5-hydroxytryptamine (5-HT) agonist provided herein.

FIG. 3 shows behavior of mice administered various doses of a 5-hydroxytryptamine (5-HT) agonist provided herein.

FIG. 4 shows the difference in ASSR brain activity (in EP (FIG. 4A) and dB EP (FIG. 4B)) of mice administered various doses of a 5-hydroxytryptamine (5-HT) agonist provided herein.

FIG. 5 shows the difference in ASSR brain activity (in PLF (FIG. 5A) and dB PLF (FIG. 5A)) of mice administered various doses of a 5-hydroxytryptamine (5-HT) agonist provided herein.

FIG. 6 shows the difference in MMN brain activity (in FC (FIG. 6A) and dB FC (FIG. 6B)) of mice administered various doses of a 5-hydroxytryptamine (5-HT) agonist provided herein.

FIG. 7 shows high frequency (FIG. 7A and FIG. 7B) brain activity of mice administered various doses of a 5-hydroxytryptamine (5-HT) agonist provided herein.

FIG. 8 shows low frequency (FIG. 8A and FIG. 8B) brain activity of mice administered various doses of a 5-hydroxytryptamine (5-HT) agonist provided herein.

FIG. 9 shows the difference in brain activity of mice stimulated with high (FIG. 9A and FIG. 9B) and low (FIG. 9C and FIG. 9D) frequency tones and administered various doses of a 5-hydroxytryptamine (5-HT) agonist provided herein.

FIG. 10 shows brain activity measured in different frequency bands.

FIG. 11 shows a Short State Trait Anxiety Inventory (STAI).

FIGS. 12A-12C show a Beck Depression Inventory (BDI).

FIGS. 13A-13B show a Depression Anxiety Stress Scale (DASS).

FIG. 14 shows a Generalized Anxiety Disorder (GAD-7) questionnaire.

FIGS. 15A-15C show a Brief-COPE questionnaire.

FIGS. 16A-16B show a Positive and Negative Affect Schedule (PANAS).

FIGS. 17A-17B show a 36-Item Short Form Health Survey (SF-36).

FIGS. 18A-18B show a mobile application as provided herein connecting users to medical and recreational marijuana.

FIG. 19 shows a mobile application as provided herein implementing artificial intelligence and machine learning.

FIG. 20 shows a morning survey provided to a user on a mobile application as provided herein.

FIG. 21 shows an evening survey including a Short State Trait Anxiety Inventory (STAI) and other questions about attention, social interactions, fatigue, and mood provided to a user on a mobile application as provided herein.

FIGS. 22A-B show the effects of psilocybin on progressive ratio (PR) test in treated rats. FIG. 22A shows number of lever presses prior to treatment with vehicle or psilocybin by high and low responder rat groups, to establish pre-test PR baseline. FIG. 22B shows number of lever presses by high and low responder rat subgroups across psilocybin doses of 0.05 mg/kg, 0.1 mg/kg and 0.2 mg/kg. High responders are defined as rats completing the highest tertile of lever presses in the baseline test; low responders are rats completing the bottom tertile of lever presses in the baseline test. Asterisk (*) indicates statistical significance between high and low responders.

FIGS. 23A-D show the effects of psilocybin on cognition in rats using the 5-choice serial reaction time task (5-CSRTT) with 5 second inter-trial interval. FIG. 23A shows pro-cognitive effects (measured as % Hit regarding nose-poke to stimulation location to collect a food reward) of psilocybin 0.05 mg/kg dose versus vehicle and 0.1 mg/kg dose across all rats. Asterisk (*) indicates statistical difference vs. vehicle. FIG. 23B shows pro-cognitive effects (measured as % Correct regarding accuracy in nose-poke) of psilocybin 0.05 mg/kg dose versus vehicle and 0.1 mg/kg dose across all rats. FIG. 23C shows pro-cognitive effect of two different doses of psilocybin on low performer subgroup (% Hit). Asterisk (*) indicates statistical difference vs, vehicle FIG. 23D shows effect of two different doses of psilocybin on low performer subgroup (% Correct). High performers are defined as rats completing the highest tertile of Hits or Correct nose-pokes in the baseline test; low performers are rats completing the bottom tertile of Hits or Correct nose-pokes in the baseline test.

FIGS. 24A-B shows the effects of psilocybin on cognition in rats using 5-choice serial reaction time task (5-CSRIT) and evaluating premature responses (PREM) and perseverative responses (PSV). FIG. 24A shows increase in PREM and PSV responses under a 5 second inter-trial interval (ITT) which establishes a baseline (Base) and a 10 second ITT across 24 animals across two doses of psilocybin (0.05 mg/kg and 0.1 mg/kg), Standard deviation is indicated by error bars; asterisks (1 indicate significance vs. vehicle (P=0,05) using T-test, FIG. 24B shows effects of psilocybin on PREM and PSV in low performer and high performer subgroups. Standard error of the mean is indicated by error bars; asterisks (*) indicate significance vs. vehicle (P<0,01) using T-test. High performers are defined as rats with the highest tertile of premature responses in the baseline test; low performers are rats with the bottom tertile of premature responses in the baseline test.

FIG. 25 shows the blood plasma levels over time of psilocin in rats dosed with psilocybin at several dose levels: 0.05 mg/kg (Cₘₐₓ psilocin ⁻6+2 ng/ml (after 30 mins)) or 0.1 mg/kg (Cmax psilocin ⁻12 3 ng/ml (after 30 mins)), 1 mg/kg (Cmax psilocin ⁻83 5 ng/ml (after 30 mins)), 10 mg/kg (Cmax psilocin ⁻1106 164 ng/ml (after 30 mins)).

FIGS. 26A-E shows the effects of psilocybin on cognition in rats. FIG. 26A shows the lowest performing tertile (N=8) low attentive and potentially representative of a low attentive endophenotype of depression. FIG. 26B shows % hit score for low attentive rats. FIG. 26D shows a slower response speed for low attentive rats. Similar to the PR test, the effect of 0.05 and 0.1 mg/kg psilocybin on accuracy (% correct and % hit) in the 5CSRTT was observed to be strongly evident in the low attentive subgroup compared with vehicle (FIG. 26C and FIG. 26E). Asterisk (*) indicates statistical significance vs. vehicle. Psilocybin 0.05 mg/kg also increased response speed in the low attentive cohort compared with vehicle (FIG. 26D).

### DETAILED DESCRIPTION

Provided in some embodiments herein are methods and compositions comprising 5-HT (5-HT _{2A}) receptor agonists, or the administration thereof (e.g., monitoring or evaluating the effect of the administration thereof (e.g., to provide a therapeutically effective dose (e.g., an optimal dose) of the 5-HT receptor agonist (e.g., over a period of time))).

### Certain Definitions

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a”, “an”, and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

The section headings used herein are for organizational purposes only and are not to be construed as limiting the subject matter disclosed.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g., constant or intermittent) of a health care worker (e.g., a doctor, a registered nurse, a nurse practitioner, a physician's assistant, an orderly or a hospice worker).

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

The terms “effective amount” or “pharmaceutically effective amount” or “therapeutically effective amount” refer to a nontoxic but sufficient amount of the agent to provide the desired biological, therapeutic, and/or prophylactic result. That result might be reduction and/or alleviation of the signs, symptoms, or causes of a disease, or any other desired alteration of a biological system. For example, an “effective amount” for therapeutic uses is the amount of a 5-HT receptor agonist or a pharmaceutically acceptable salt, solvate, metabolite, derivative, or prodrug thereof as disclosed herein per se or a composition comprising a 5-HT receptor agonist or a pharmaceutically acceptable salt, solvate, metabolite, derivative, or prodrug thereof as disclosed herein required to provide a clinically significant decrease in a disease. An appropriate effective amount in any individual case might be determined by one of ordinary skill in the art using routine experimentation.

The term “5-HT receptor agonist agent” refers to a 5-HT receptor agonist as a free base or a derivate or analog thereof. Included in the term are salts, solvates, metabolites, prodrugs, isomers, tautomers, isotopic derivatives, and the like, of a 5-HT receptor agonist. In some embodiments, the derivates, analogs, salts, solvates, metabolites, prodrugs, isomers, tautomers, isotopic derivatives, etc are pharmaceutically acceptable derivates, analogs, salts, solvates, metabolites, prodrugs, isomers, tautomers, isotopic derivatives of a 5-HT receptor agonist.

The term “pharmaceutically acceptable,” as used herein, refers a material, such as a carrier or diluent, which does not abrogate the biological activity or properties of the compound, and is relatively nontoxic, i.e., the material is administered to an individual without causing undesirable biological effects or interacting in a deleterious manner with any of the components of the composition in which it is contained.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties, Selection and Use. International Union of Pure and Applied Chemistry, Wiley-VCH 2002. S. M. Berge, L. D. Bighley, D. C. Monkhouse, J. Pharm. Sci. 1977, 66, 1-19. P. H. Stahl and C. G. Wermuth, editors, Handbook of Pharmaceutical Salts: Properties, Selection and Use, Weinheim/Zürich:Wiley-VCH/VHCA, 2002. Pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible and this capability can be manipulated as one aspect of delayed and sustained release behaviors. Also, because the salt-forming molecule can be in equilibrium with a neutral form, passage through biological membranes can be adjusted.

In some embodiments, pharmaceutically acceptable salts are obtained by reacting a compound described herein with an acid to provide a “pharmaceutically acceptable acid addition salt.” In some embodiments, the compound described herein (i.e., free base form) is basic and is reacted with an organic acid or an inorganic acid. Inorganic acids include, but are not limited to, hydrochloric acid, hydrobromic acid, metaphosphoric acid, nitric acid, phosphoric acid, and sulfuric acid. Organic acids include, but are not limited to, 1-hydroxy-2-naphthoic acid; 2,2-dichloroacetic acid; 2-hydroxyethanesulfonic acid; 2-oxoglutaric acid; 4-acetamidobenzoic acid; 4-aminosalicylic acid; acetic acid; adipic acid; ascorbic acid (L); aspartic acid (L); benzenesulfonic acid; benzoic acid; camphoric acid (+); camphor-10-sulfonic acid (+); capric acid (decanoic acid); caproic acid (hexanoic acid); caprylic acid (octanoic acid); carbonic acid; cinnamic acid; citric acid; cyclamic acid; dodecylsulfuric acid; ethane-1,2-disulfonic acid; ethanesulfonic acid; formic acid; fumaric acid; galactaric acid; gentisic acid; glucoheptonic acid (D); gluconic acid (D); glucuronic acid (D); glutamic acid; glutaric acid; glycerophosphoric acid; glycolic acid; hippuric acid; isobutyric acid; lactic acid (DL); lactobionic acid; lauric acid; maleic acid; malic acid (−L); malonic acid; mandelic acid (DL); methanesulfonic acid; monomethyl fumarate, naphthalene-1,5-disulfonic acid; naphthalene-2-sulfonic acid; nicotinic acid; oleic acid; oxalic acid; palmitic acid; pamoic acid; proprionic acid; pyroglutamic acid (−L); salicylic acid; sebacic acid; stearic acid; succinic acid; sulfuric acid; tartaric acid (+L); thiocyanic acid; toluenesulfonic acid (p); and undecylenic acid.

In some embodiments, a compound described herein is prepared as a chloride salt, sulfate salt, bromide salt, mesylate salt, maleate salt, citrate salt or phosphate salt.

In some embodiments, pharmaceutically acceptable salts are obtained by reacting a compound described herein with a base to provide a “pharmaceutically acceptable base addition salt”. In some embodiments, the compound described herein is acidic and is reacted with a base. In such situations, an acidic proton of the compound described herein is replaced by a metal ion, e.g., lithium, sodium, potassium, magnesium, calcium, or an aluminum ion. In some cases, compounds described herein coordinate with an organic base, such as, but not limited to, ethanolamine, diethanolamine, triethanolamine, tromethamine, meglumine, N-methylglucamine, dicyclohexylamine, tris(hydroxymethyl)methylamine. In other cases, compounds described herein form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, potassium carbonate, sodium hydroxide, lithium hydroxide, and the like. In some embodiments, the compounds provided herein are prepared as a sodium salt, calcium salt, potassium salt, magnesium salt, meglumine salt, N-methylglucamine salt or ammonium salt.

In additional or further embodiments, the compounds described herein are metabolized upon administration to an organism in need to produce a metabolite that is then used to produce a desired effect, including a desired therapeutic effect.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes might produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulphydryl groups. Metabolites of the compounds disclosed herein are optionally identified either by administration of compounds to a host and analysis of tissue samples from the host, or by incubation of compounds with hepatic cells in vitro and analysis of the resulting compounds.

The term “treating” and its grammatical equivalents as used herein include achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding the fact that the patient might still be afflicted with the underlying disorder. For prophylactic benefit, a method might be performed on, or a composition might be administered to a patient at risk of developing a disease, or to a patient reporting one or more of the physiological symptoms of such conditions, even though a diagnosis of the condition might not have been made.

### 5-HT (or Serotonin) RECEPTORS

The 5-HT (or serotonin) receptors area group of G protein-coupled receptors (GPCR) and ligand-gated ion channels. 5-HT is short for 5-hydroxy-tryptamine, the chemical name for serotonin,

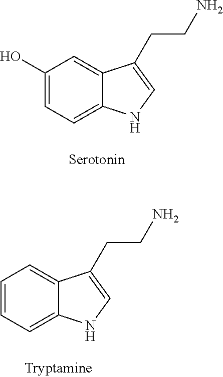 [SMILES: NCCc1c[nH]c2ccc(O)cc12 ; NCCc1c[nH]c2ccccc12]

The serotonin receptors are activated by serotonin, their natural ligand, and mediate both excitatory and inhibitory neurotransmission. They modulate the release of many neurotransmitters, including glutamate, GABA, dopamine, epinephrine norepinephrine and acetylcholine, as well as many hormones, including oxytocin, prolactin, vasopressin, cortisol, corticotropin and substance P. The serotonin receptors influence various biological and neurological processes such as aggression, anxiety, appetite, cognition, learning, memory, mood, nausea, sleep, and thermoregulation.

The 5-HT receptors are divided into 7 families of G protein-coupled receptors. 5-HT₁, 5-HT₂, 5-HT3 are the major families; the others, 5-HT₄, 5-HT₅, 5-HT₆ and 5-HT₇, for the most part, work in a similar fashion to either 5-HT₁ or 5-HT₂ receptors. The 5-HT receptors work with a G protein to modify an ion channel or membrane enzyme.

In certain instances, the 5-HT agonist of a formulation, composition, method, or the like described herein is a 5-HT₁ agonist. 5-HT₁ receptors have strong binding affinity for serotonin. Typically, when serotonin binds to a 5-HT₁ receptor, a G-protein is activated, opening an ion channel and allowing potassium ions to exit the neuron. This generally causes the neuron to become more negatively charged, making it more difficult to trigger an action potential, i.e., serotonin binding to 5-HT₁ receptors is an inhibitory effect.

In some preferred embodiments, the 5-HT agonist of a formulation, composition, method, or the like described herein is a 5-HT₂ agonist. In certain instances, the 5-HT₂ agonist has a relatively high affinity for 5-HT₂ receptors (e.g., relative to 5-HT₁ receptors and/or other 5-HT receptors, such as 5-HT₃, 5-HT₄, 5-HT s, 5-HT₆, 5-HT₇, or all or some combination thereof, such as 2×, 3×, 5×, 10×, 20×, 50×, or the like greater affinity). 5-HT₂ receptors have weaker affinity for serotonin. As such, serotonin prefers to bind 5-HT₁ receptors, typically only binding 5-HT₂ receptors once the 5-HT₁. receptors are at least partially (or wholly) saturated. Serotonin binding of 5-HT₂ receptors typically activates a G-protein closing a potassium channel resulting in potassium ion build up. This generally causes depolarization, making it easier to reach the neuron's excitation threshold. Thus, when serotonin binds to 5-I-IT 2 receptors, it typically has an excitatory effect.

| Family | Type | Mechanism | Potential |
|---|---|---|---|
| 5-HT₁ | Protein coupled | Decreasing cellular levels of cAMP | Inhibitory |
| 5-HT₂ | Protein coupled | Increasing cellular levels of IP₃ and | Excitatory |
| | | DAG | |
| 5-HT₃ | Ligand-gated Na⁺ and K⁺ | Depolarizing plasma membrane | Excitatory |
| | cation channel | | |
| 5-HT₄ | Protein coupled | Increasing cellular levels of cAMP | Excitatory |
| 5-HT₅ | Protein coupled | Decreasing cellular levels of cAMP | Inhibitory |
| 5-HT₆ | Protein coupled | Increasing cellular levels of cAMP | Excitatory |
| 5-HT₇ | Protein coupled | Increasing cellular levels of cAMP | Excitatory |

The seven serotonin receptor families include fourteen receptor subtypes, distributed throughout the body as shown in the table below:

| | | Central | Peripheral | | | |
|---|---|---|---|---|---|---|
| 5HT | Blood | nervous | nervous | | | Smooth |
| Receptor | vessels | system | system | GI Tract | Platelets | Muscle |
| 1A | X | X | | | | |
| 1B | X | X | | | | |
| 1D | X | X | | | | |
| 1E | X | X | | | | |
| 1F | | X | | | | |
| 2A | X | X | X | X | X | X |
| 2B | X | X | X | X | X | X |
| 2C | X | X | X | X | X | X |
| 3 | | X | X | X | | |
| 4 | | X | X | X | | |
| 5A | | X | | | | |
| 5B | | | | | | |
| 6 | | X | | | | |
| 7 | X | X | | X | | |

### 5-HT₂ RECEPTORS

In general, 5-HT₂ receptors are characterized by having lower affinity for serotonin (and other indolealkylamines), and are linked to the G_{q}/phospholipase C pathway of signal transduction. In various instances, such receptors mediate a variety of physiological and behavioral functions via three distinct subtypes: 5-HT_{2A}, 5-HT_{2B} and 5-HT_{2C}.

| Receptor | |
|---|---|
| | Physiological/behavioral function |
| 5-HT_{2A} | Addiction, Anxiety, Apathy, Appetite, Cognition, Depression, Imagination, |
| | Learning, Memory, Mood, Perception, Sexual Behavior, Sleep, |
| | Thermoregulation, Vasoconstriction |
| 5-HT_{2B} | Anxiety, Appetite, Cardiovascular Function, GI Motility, Sleep, |
| | Vasoconstriction |
| 5-HT_{2C} | Addiction, Anxiety, Appetite, GI Motility, Locomotion, Mood, Penile Erection, |
| | Sexual Behavior, Sleep, Thermoregulation, Vasoconstriction |
| | Uses of drugs that act on this receptor |
| 5-HT_{2A} | Antipsychotics, Psychedelics, Noradrenergic and Specific Serotonergic |
| | Antidepressants (NaSSAs), Sleeping aids |
| 5-HT_{2B} | Migraines |
| 5-HT_{2C} | Antidepressant, Orexigenic, Anorectic, Antipsychotic |
| | Agonists acting on receptor |
| 5-HT_{2A} | Bufotenin, Ergonovine, Lisuride, LSD, Mescaline, Myristicin, Psilocin, |
| | Psilocybin, DMT, DOM, PNU-22394, TFMPP, 25I-NBOMe, 2C-B, 5-MeO- |
| | DMT, BZP |
| 5-HT_{2B} | Fenfluramine, MDMA, Norfenfluramine, Methylphenidate |
| | 6-APB, BW-723C86, PNU-22394, Ro60-0175 |
| 5-HT_{2C} | Aripiprazole, Ergonovine, Lorcaserin, Trazodone |
| | PNU-22394, Ro60-0175, TFMPP, YM-348, A-372,159, AL-38022A |

5-HT_{2A} is an important excitatory serotonin receptor subtype. In some instances, physiological processes mediated by the receptor include, by way of non-limiting example:
central nervous system—neuronal excitation, behavioral effects, learning, anxiety, and pro-nociception
smooth muscle contraction (in bronchi and gastrointestinal tract)
vasoconstriction/vasodilation
platelet aggregation
role in memory and learning
anti-inflammatory activity
hormone (oxytocin, prolactin, ACTH, corticosterone, renin) regulation
mood regulation (depressed patients have more 5-HT_{2A} receptors than otherwise normal individuals implying 5-HT_{2A} is involved in the pathogenesis of depression)

In some instances, agonism of 5-HT_{2A} agonism facilitates treatment or management of disorders involving cognitive function and social interaction, or the symptoms thereof, as evidenced by the extensive localization of the 5-HT_{2A} receptor in brain areas that mediate cognitive functions and social interaction. In some instances, disorders in which the 5-HT_{2A} receptor are involved include, but are not limited to schizophrenia, apathy, depression suicide (e.g., low motivation), anxiety, obsessive compulsive disorders (OCL), bipolar disorders, attention deficit hyperactivity disorder (ADHD), eating disorders such as anorexia nervosa, autism and autism spectrum disorders, Asperger's, neuropsychiatric diseases and disorders, sexual disorders such as erectile dysfunction, neurodegenerative diseases, inflammatory diseases, autoimmune diseases, metabolic diseases such as obesity and diabetes, central nervous system disorders, peripheral nervous system disorders, Alzheimer's disease, snoring, sleep apnea (obstructive sleep apnea, central sleep apnea), insomnia, sleep deprivation, restless legs syndrome, parasomnia, nightmares, night terrors, sleepwalking, hypersomnia (daytime sleepiness), narcolepsy and pain.

Any suitable 5-HT (e.g., 5-HT₂, such as 5-HT_{2A}) agonist is utilized in any composition, formulation, method, therapy, or the like described herein. In some preferred embodiments, the 5-HT agonist of a formulation, composition, method, or the like described herein is a 5-HT_{2A} agonist. In certain instances, the 5-HT_{2A} agonist has a relatively high affinity for 5-HT_{2A} receptors (e.g., relative to 5-HT₁, 5-HT₃, 5-HT₄, 5-HT₅, 5-HT₆, 5-HT₇, 5-HT_{2B}, 5-HT_{2C}, or all or some combination thereof, such as 2×, 3×, 5×, 10×, 20×, 50×, or the like greater affinity). In some instances, 5-HT_{2A} agonists increase dopamine levels in the prefrontal cortex. In certain instances, the 5-HT_{2A} agonist provided herein is one of the following classes of 5-HT_{2A} agonists: the ergolines, tryptamines and phenethylamines.

### Ergolines

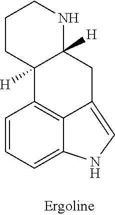 [SMILES: c1cc2c3c(c[nH]c3c1)C[C@H]1NCCC[C@H]21]

In specific embodiments, a 5-HT (e.g., 5-HT_{2A}) receptor agonist utilized herein is an ergoline. In some instances, ergonovine and ergotamine, synthetic derivatives include the oxytocic methergine, the anti-migraine drugs dihydroergotamine and methysergide, hydergine (a mixture of dihydroergotoxine mesylates, INN: ergoline mesylates), and bromocriptine. In certain instances, synthetic ergolines include pergolide and lisuride.

In certain instances, the ergoline is an ergoline derivative, such as a lysergic acid amide or a peptide alkaloid, such as described below. In some instances, the ergoline isa clavine (examples include festuclavine, fumigaclavine A, fumigaclavine B and fumigaclavine C) and other derivatives that do not fall into these categories, such as cabergoline, pergolide, lisuride.

### Lysergic Acid Amides

Exemplary lysergic acid amides include Ergine (LSA, D-lysergic acid amide), Ergonovine (ergobasine), Methergine (ME-277), Methysergide (UML-491), LSD (D-lysergic acid diethylamide), LSH (0-lysergic acid α-hydroxyethylamid. The table below summarizes their structural formula and relationships.

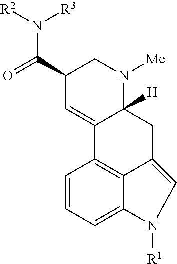

| Name | R¹ | R² | R³ |
|---|---|---|---|
| Ergine | H | H | H |
| Ergonovine | H | CH(CH₃)CH₂OH | H |
| Methergine | H | CH(CH₂CH₃)CH₂OH | H |
| Methysergide | CH₃ | CH(CH₂CH₃)CH₂OH | H |
| LSD | H | CH₂CH₃ | CH₂CH₃ |
| LSH | H | CH(OH)CH₃ | H |

### Peptide Alkaloids

Exemplary peptide alkaloids include, peptide ergot alkaloids (ergopeptines or ergopeptides), which are ergoline derivatives containing a tripeptide structure (attached at the same position as the amide group of the lysergic acid derivatives) comprising proline and two other α-amino acids. Examples include:
Ergotoxines (valine at R²)—Ergocristine, Ergocornine, α-Ergocryptine, β-Ergocryptine
Ergotamines (alanine at R²)—Ergotamine, Ergovaline, α-Ergosine, β-Ergosine.

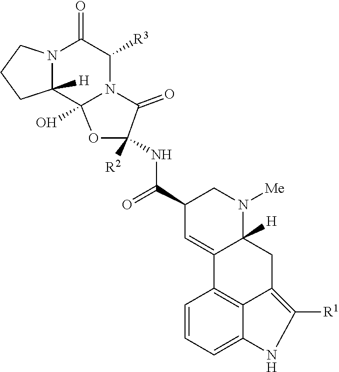

| Name | R¹ | R² | R³ | R³ Amino acid |
|---|---|---|---|---|
| Ergocristine | | CH(CH₃)₂ | benzyl | Phenylalanine |
| Ergocornine | | CH(CH₃)₂ | CH(CH₃)₂ | Valine |
| α-Ergocornine | | CH(CH₃)₂ | CH₂CH(CH₃)₂ | Leucine |
| β-Ergocryptine | | CH(CH₃)₂ | (S)-CH(CH₃)CH₂CH₃ | Isoleucine |
| Ergotamine | | CH₃ | benzyl | Phenylalanine |
| Ergovaline | | CH₃ | CH(CH₃)₂ | Valine |
| α-Ergosine | | CH₃ | CH₃CH₂(CH₃)₂ | Leucine |
| β-Ergosine | | CH₃ | (S)-CH(CH₃)CH₂CH₃ | Isoleucine |
| Bromocriptin | Br | CH(CH₃)₂ | CH₂CH(CH₃)₂ | Leucine |

### Tryptamines

Tryptamine (2-(1H-Indol-3-yl)ethanamine) comprises an indole ring, attached to an aminoethylene group; substituted tryptamines are substituted with any suitable group, such as being modified on the indole ring (R¹, R²), the ethylene chain (R³) and/or on the amino group (R⁴, R⁵), as illustrated below, and are collectively referred to herein as tryptamines. Examples of tryptamines include serotonin, melatonin, psilocybin and N,N-Dirnethyltryptarnine. Additionally, the tryptamine structure may comprise part of a more complex compound, for example: LSD, ibogaine, mitragynine, yohimbine, etc.

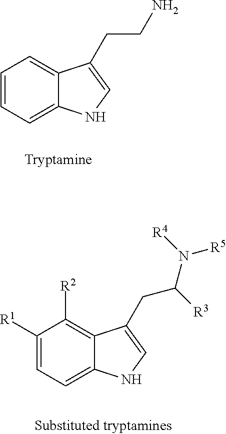

Examples of naturally occurring substituted tryptamines include, by way of non-limiting example:

| Short/Common | | | | | | |
|---|---|---|---|---|---|---|
| Name | Full Name | R¹ | R² | R³ | R⁴ | R⁵ |
| Tryptamine | 3-(2-aminoethyl)indole | H | H | H | H | H |
| | 2-(1H-indol-3- | | | | | |
| | yl)ethanamine | | | | | |
| Bufotenin | 5-hydroxy-N,N- | OH | H | H | CH₃ | CH₃ |
| | dimethyltryptamine | | | | | |
| Nω- | 5-hydroxy-N- | OH | H | H | CH₃ | H |
| Methylserotonin | methyltryptamine | | | | | |
| (norbufotenin) | | | | | | |
| Serotonin | 5-hydroxytryptamine | OH | H | H | H | H |
| NMT | N-methyltryptamine | H | H | H | H | CH₃ |
| 5-MeO-NMT | 5-methoxy-N- | OCH₃ | H | H | CH₃ | H |
| | methyltryptamine | | | | | |
| DMT | N,N-dimethyltryptamine | H | H | H | CH₃ | CH₃ |
| 5-Bromo-DMT | 5-bromo-N,N- | Br | H | H | CH₃ | CH₃ |
| | dimethyltryptamine | | | | | |
| 5-MeO-DMT | 5-methoxy-N,N- | OCH₃ | H | H | CH₃ | CH₃ |
| | dimethyltryptamine | | | | | |
| Melatonin | 5-methoxy-N- | OCH₃ | H | H | C(O)CH₃ | H |
| | acetyltryptamine | | | | | |
| N- | 5-hydroxy-N- | OH | H | H | C(O)CH₃ | H |
| Acetylserotonin | acetyltryptamine | | | | | |
| Norbaeocystin | 4-phosphoryloxy- | H | OPO₃H₂ | H | H | H |
| | tryptamine | | | | | |
| Baeocystin | 4-phosphoryloxy-N- | H | OPO₃H₂ | H | CH₃ | H |
| | methyl-tryptamine | | | | | |
| Psilocybin | 4-phosphoryloxy-N,N- | H | PO₄ | H | CH₃ | CH₃ |
| | dimethyltryptamine | | | | | |
| Psilocin | 4-hydroxy-N,N- | H | OH | H | CH₃ | CH₃ |
| | dimethyltryptamine | | | | | |
| Tryptophan | α-carboxyltryptamine | H | H | COOH | H | H |

Examples of synthetic substituted tryptamines include, by way of non-limiting example:

| Short | | | | | | |
|---|---|---|---|---|---|---|
| Name | Name | R¹ | R² | R³ | R⁴ | R⁵ |
| αET | α-ethyltryptamine | H | H | CH₂CH₃ | H | H |
| αMT | α-methyltryptamine | H | H | CH₃ | H | H |
| DALT | N,N-diallyltryptamine | H | H | H | H₂C═CH—CH₂ | H₂C═CH—CH₂ |
| DET | N,N-diethyltryptamine | H | H | H | CH₂CH₃ | CH₂CH₃ |
| DiPT | N,N- | H | H | H | CH(CH₃)₂ | CH(CH₃)₂ |
| | diisopropyltryptamine | | | | | |
| DPT | N,N-dipropyltryptamine | H | H | H | CH₂CH₂CH₃ | CH₂CH₂CH₃ |
| 5-MeO-αMT | 5-methoxy-α- | OCH₃ | H | CH₃ | H | H |
| | methyltryptamine | | | | | |
| 5-MeO-DALT | 5-methoxy-N,N- | OCH₃ | H | H | H₂C═CH—CH₂ | H₂C═CH—CH₂ |
| | diallyltryptamine | | | | | |
| 5-MeO-MALT | 5-methoxy-N-Methyl-N- | OCH₃ | H | H | H₂C═CH—CH₂ | CH₃ |
| | allyltryptamine | | | | | |
| 4-HO-DET | 4-hydroxy-N,N- | H | OH | H | CH₂CH₃ | CH₂CH₃ |
| | diethyltryptamine | | | | | |
| 4-AcO-DMT | 4-acetoxy-N,N- | H | OCOCH₃ | H | CH₃ | CH₃ |
| | dimethyltryptamine | | | | | |
| 4-HO-MET | 4-hydroxy-N-methyl-N- | H | OH | H | CH₃ | CH₂CH₃ |
| | ethyltryptamine | | | | | |
| 4-HO-DIPT | 4-hydroxy-N,N- | H | OH | H | CH(CH₃)₂ | CH(CH₃)₂ |
| | diisopropyltryptamine | | | | | |
| 5-MeO-DIPT | 5-methoxy-N,N- | OCH₃ | H | H | CH(CH₃)₂ | CH(CH₃)₂ |
| | diisopropyltryptamine | | | | | |
| 5-MeO-MiPT | 5-methoxy-N,N- | OCH₃ | H | H | CH₃ | CH(CH₃)₂ |
| | methylisopropyltryptamine | | | | | |
| 4-HO-MiPT | 4-hydroxy-N-isopropyl-N- | H | OH | H | CH(CH₃)₂ | CH₃ |
| | methyltryptamine | | | | | |
| Sumatriptan | 5-(methylamino | CH₂SO₂NHCH₃ | H | H | CH₃ | CH₃ |
| | sulfonylmethylene)-N,N- | | | | | |
| | dimethyltryptamine | | | | | |
| Zolmitriptan | 5-(4-(S)-1,3-oxazolidin-2- | CHNHC(O)OCH₂ | H | H | CH₃ | CH₃ |
| | one)-N,N- | | | | | |
| | dimethyltryptamine | | | | | |

### Phenethylamines

Phenethylamine comprises a phenyl ring attached to an aminoethylene group; substituted phenethylamines are optionally substituted in any suitable manner, such as they are optionally modified by substitution on the phenyl ring (R¹, R², R³, and/or R⁵), the ethylene chain (R⁶ and/or R⁷) and/or on the amino group (R⁸ and/or R⁹), such as illustrated below.

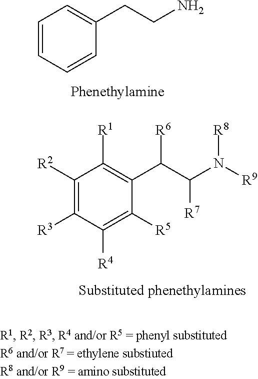

Examples of phenethylamines include, but are not limited those presented in the table below:

| | | Substitution | | | Biological |
|---|---|---|---|---|---|
| Short Name | Full Name | Amino | Ethylene | Phenyl | activity |
| Amphetamine | α- | | X | | Stimulant |
| | methylphenethylamine | | | | |
| β-Methyl | β- | | X | | Stimulant |
| phenethylamine | methylphenethylamine | | | | |
| Mephedrone | 4-methylmethcathinone | X | X | X | Stimulant |
| Ethcathinone | N-ethylcathinone | X | X | | Stimulant |
| Ephedrine/ | N-methyl-β- | X | X | | Stimulant; |
| Pseudoephedrine | hydroxyamphetamine | | | | decongestant |
| Methamphetamine | N-methylamphetamine | X | X | | Stimulant; |
| | | | | | neurotoxin |
| Phentermine | α-methylamphetamine | | X | | Stimulant, |
| | | | | | anorectic |
| Ortetamine | 2-methylamphetamine | | X | X | Stimulant, |
| | | | | | anorectic |
| Amfepramone | N-diethyl-β- | X | X | | Anorectic |
| (diethylpropion) | ketoamphetamine | | | | |
| Phenylephrine | β,3-dihydroxy-N- | X | X | X | Decongestant |
| | methylphenethylamine | | | | |
| Methylphenidate | N,α-butylene-β- | X | X | | Stimulant; NDRI |
| | methoxy | | | | |
| | carbonylphenethylamine | | | | |
| Dopamine | 3,4- | | | X | Catecholamine |
| | dihydroxyphenethylamine | | | | neurotransmitter |
| 6- | 2,4,5-trihydroxy | | | X | Neurotoxic |
| Hydroxydopamine | phenethylamine | | | | agent |
| Epinephrine | β-3,4-trihydroxy-N- | X | X | X | Catecholamine |
| (Adrenaline) | methylphenethylamine | | | | neurotransmitter |
| Norepinephrine | β-3,4-trihydroxy | | X | X | Catecholamine |
| (Noradrenaline) | phenethylamine | | | | neurotransmitter |
| para-Octopamine | β-4-dihydroxy | | X | X | Trace aminergic |
| | phenethylamine | | | | α-adrenoceptor |
| | | | | | agonist |
| Salbutamol | β-4-dihydroxy-3- | X | X | X | Short-action β2- |
| | hydroxymethyl-N-tert- | | | | adrenergic |
| | butylphenethylamine | | | | agonist |
| N-Methyl | N- | X | | | Amphetamine |
| phenethylamine | methylphenethylamine | | | | isomer |
| Cathine | d-β- | | X | | Releasing agent |
| | hydroxyamphetamine | | | | |
| Cathinone | β-ketoamphetamine | | X | | Releasing agent |
| Methcathinone | N-methylcathinone | X | X | | Releasing agent |
| Bupropion | 3-chloro-N-tert-butyl-β- | X | X | X | NDRI |
| | ketoamphetamine | | | | |
| Norfenfluramine | 3-trifluoromethyl- | | X | X | SSRA |
| | amphetamine | | | | |
| Fenfluramine | 3-trifluoromethyl-N- | X | X | X | SSRA |
| | ethylamphetamine | | | | |
| Mescaline | 3,4,5-trimethoxy | | | X | Psychedelic |
| | phenethylamine | | | | |
| Proscaline | 2-(3,5-dimethoxy-4- | | | X | Psychedelic |
| | propoxyphenyl)ethanamine | | | | |
| Metaescaline | 2-(3-ethoxy-4,5- | | | X | Psychedelic |
| | dimethoxyphenyl)ethanamine | | | | |
| Allylescaline | 4-Allyloxy-3,5- | | | X | Psychedelic |
| | dimethyloxyphenylethylamine | | | | |
| Methallylescaline | 4-Methallyloxy-3,5- | | | X | Psychedelic |
| | dimethoxyphenethylamine | | | | |
| Asymbescaline | 3,4-Diethoxy-5- | | | X | Psychedelic |
| | methoxyphenethylamine | | | | |

In some embodiments, described herein are pharmaceutical compositions, comprising 5-HT receptor agonist, or pharmaceutically acceptable salt, solvate, metabolite, derivative, or prodrug thereof, such agents being collectively referred to herein as 5-HT receptor agonist agents. In some instances, the pharmaceutical formulations of 5-HT receptor agonist, or pharmaceutically acceptable salt, solvate, metabolite, derivative, or prodrug thereof, have enhanced bioavailability and efficacy, have a lower administration dose, a lower cytotoxicity, and/or have decreased side effects.

In various embodiments provided herein, any suitable route of administration is contemplated. In specific embodiments, the composition is formulated for oral, buccal, nasal or inhalation administration. In some embodiments, the composition is an oral, buccal, nasal or inhalation composition.

In specific embodiments, the composition further comprises any suitable (e.g., pharmaceutically acceptable) excipients and/or additives, such as surfactants, preservatives, flavoring agents, sweetening agents, or anti-foaming agents.

Any suitable composition, formulation, or dosage form is contemplated herein. In some embodiments, the composition, formulation, or dosage form is an oral composition, formulation or dosage form. In some specific embodiments, the pharmaceutically acceptable excipient is selected from the group consisting of fillers, binders, suspending agents, disintegrants, lubricants, and combinations thereof.

Provided herein are methods for managing disorders or conditions, comprising administering one or more 5-HT receptor agonists, or pharmaceutically acceptable salts, solvates, metabolites, derivatives, or prodrugs thereof.

Further provided herein are methods for treating symptoms of disorders or conditions, comprising administering one or more 5-HT receptor agonists, or pharmaceutically acceptable salts, solvates, metabolites, derivatives, or prodrugs thereof.

Further provided herein is a method of treating any disorder or condition treatable by 5-HT receptor agonism.

Provided in some embodiments herein is a method for treating or reducing the incidence of a brain disorder, comprising administering one or more 5-HT receptor agonists, or pharmaceutically acceptable salts, solvates, metabolites, derivatives, or prodrugs thereof.

Provided in some embodiments herein is a method for treating or reducing the incidence of a brain disorder, comprising titrating one or more 5-HT receptor agonists, or pharmaceutically acceptable salts, solvates, metabolites, derivatives, or prodrugs thereof. In some embodiments, titrating comprises (e.g., sequentially) adjusting the amount(s) of one or more 5-HT receptor agonist, or pharmaceutically acceptable salt, solvate, metabolite, derivative, or prodrug thereof administered to an individual, such as, until a desired (e.g., neurological) effect (e.g., neurological effect as measured by one or more brain evaluation (e.g., one or more EEG)) is established in the individual. In some embodiments, titrating comprises (e.g., sequentially) adjusting the amount(s) of one or more 5-HT receptor agonist, or pharmaceutically acceptable salt, solvate, metabolite, derivative, or prodrug thereof administered to an individual, such as, until a therapeutically effective dose is established. In some embodiments, titrating comprises (e.g., sequentially) adjusting the amount(s) of one or more 5-HT receptor agonist, or pharmaceutically acceptable salt, solvate, metabolite, derivative, or prodrug thereof administered to an individual, such as, until a desired plasma level or profile of one or more 5-HT receptor agonist, or pharmaceutically acceptable salt, solvate, metabolite, derivative, or prodrug thereof is established in the individual. In some embodiments, titrating comprises (e.g., sequentially) adjusting the amount(s) of one or more 5-HT receptor agonist, or pharmaceutically acceptable salt, solvate, metabolite, derivative, or prodrug thereof administered to an individual, such as, until a desired clinical outcome is established for the individual. In some embodiments, the amount(s) of one or more 5-HT receptor agonist, or pharmaceutically acceptable salt, solvate, metabolite, derivative, or prodrug thereof is adjusted as an ascending dose or a descending dose (e.g., a combination of ascending or descending doses). In some embodiments, the amount(s) of one or more 5-HT receptor agonist, or pharmaceutically acceptable salt, solvate, metabolite, derivative, or prodrug thereof remains the same for two or more (e.g., sequential) doses. In some embodiments, of the amount of the one or more 5-HT receptor agonists, or pharmaceutically acceptable salts, solvates, metabolites, derivatives, or prodrugs thereof administered (e.g., titrated) in the individual is (at least in part) determined by a measurable neurological effect, such as, by performing one or more brain evaluation (e.g. EEG) (e.g., throughout the titration period).

Provided in some embodiments herein is a method for treating or reducing the incidence of a mental, a behavioral, or a neuropsychiatric condition, or the symptoms thereof, comprising titrating one or more 5-HT receptor agonists, or pharmaceutically acceptable salts, solvates, metabolites, derivatives, or prodrugs thereof.

Provided in some embodiments herein is a method for treating or reducing the incidence of a mental, a behavioral, or a neuropsychiatric condition, and/or the symptoms thereof, in an individual (e.g., in need thereof), comprising:
a) identifying or measuring a first brain activity of the individual by performing a brain evaluation (e.g., an electroencephalogram (EEG)) on the individual;
b) administering to the individual a first (e.g., therapeutically effective) amount of one or more 5-hydroxytryptamine (5-HT) receptor agonist (e.g., psilocybin), or a pharmaceutically acceptable salt, solvate, metabolite, derivative, or prodrug thereof;
c) identifying or measuring a second brain activity by performing another brain evaluation (e.g., another electroencephalogram (EEG)) on the individual subsequent to administering the first amount of the one or more 5-HT receptor agonist (e.g., psilocybin), or a pharmaceutically acceptable salt, solvate, metabolite, derivative, or prodrug thereof; and
d) administering to the individual a second (e.g., therapeutically effective) amount of one or more 5-HT receptor agonist (e.g., psilocybin), or a pharmaceutically acceptable salt, solvate, metabolite, derivative, or prodrug thereof (e.g., thereby treating or managing the mental, the behavioral, or the neuropsychiatric condition, and/or the symptoms thereof, in the individual).

Provided in some embodiments herein is a method for improving (e.g., psychological) coping (e.g., improving stress recovery rate) in an individual (e.g., in need thereof) comprising:
a) identifying or measuring a first brain activity of the individual by performing a brain evaluation (e.g., an electroencephalogram (EEG)) on the individual;
b) administering to the individual a first (e.g., therapeutically effective) amount of one or more 5-hydroxytryptamine (5-HT) receptor agonist (e.g., psilocybin), or a pharmaceutically acceptable salt, solvate, metabolite, derivative, or prodrug thereof;
c) identifying or measuring a second brain activity by performing another brain evaluation (e.g., another electroencephalogram (EEG)) on the individual subsequent to administering the first amount of the one or more 5-HT receptor agonist (e.g., psilocybin), or a pharmaceutically acceptable salt, solvate, metabolite, derivative, or prodrug thereof; and
d) administering to the individual a second (e.g., therapeutically effective) amount of the one or more 5-HT receptor agonist (e.g., psilocybin), or the pharmaceutically acceptable salt, solvate, metabolite, derivative, or prodrug thereof (e.g., thereby improving (e.g., psychological) coping (e.g., improving stress recovery rate) in the individual).

In some embodiments, the individual is suffering from or susceptible to stress.

Provided in some embodiments herein is a method for increasing motivation in an individual (e.g., in need thereof), comprising:
a) identifying or measuring a first brain activity of the individual by performing a brain evaluation (e.g., an electroencephalogram (EEG)) on the individual;
b) administering to the individual a first (e.g., therapeutically effective) amount of one or more 5-hydroxytryptamine (5-HT) receptor agonist (e.g., psilocybin), or a pharmaceutically acceptable salt, solvate, metabolite, derivative, or prodrug thereof;
c) identifying or measuring a second brain activity by performing another brain evaluation (e.g., another electroencephalogram (EEG)) on the individual subsequent to administering the first amount of the one or more 5-HT receptor agonist (e.g., psilocybin), or the pharmaceutically acceptable salt, solvate, metabolite, derivative, or prodrug thereof; and
d) administering to the individual a second (e.g., therapeutically effective) amount of the one or more 5-HT receptor agonist (e.g., psilocybin), or a pharmaceutically acceptable salt, solvate, metabolite, derivative, or prodrug thereof (e.g., wherein the second amount of the one or more 5-HT receptor agonist (e.g., psilocybin) is sufficient to maintain or elicit a (e.g., measurable (e.g., significant)) change in brain activity (e.g., between the first brain activity measurement and the second brain activity measurement) (e.g., thereby improving motivation in the individual).

In some embodiments, the individual is suffering from or susceptible to low motivation (e.g., low motivation induced by stress or anxiety), anxiety, apathy, fear, phobia, constructive impulsivity, or depression). In some embodiments, the individual is suffering from or susceptible to addiction.

In some embodiments, the method provided herein is a method for treating attention deficit hyperactivity disorder (ADHD) or attention deficit disorder (ADD) in an individual (e.g., in need thereof).

In some embodiments, the method provided herein is a method for treating mild cognitive impairment, dementia, or Alzheimer's disease in an individual (e.g., in need thereof).

In some embodiments, the method provided herein is a method for treating addiction (e.g., kleptomania, pyromania, addictive disorders, substance dependence, substance abuse, alcoholism, drug addiction, opioid addiction, cocaine addiction, gambling addiction, tobacco dependence, food addiction, or the like) in an individual (e.g., in need thereof).

Provided in some embodiments herein is a method for treating brain inflammation (e.g., encephalitis) or brain fog (e.g., associated with concussion, traumatic brain injury (TBI) (e.g., mild TBI (mTBI) (e.g., following (natural) insult (e.g., injury or stroke)), in an individual (e.g., in need thereof), comprising:
a) identifying or measuring a first brain activity of the individual by performing a brain evaluation (e.g., an electroencephalogram (EEG)) on the individual;
b) administering to the individual a first (e.g., therapeutically effective) amount of one or more 5-hydroxytryptamine (5-HT) receptor agonist (e.g., psilocybin), or a pharmaceutically acceptable salt, solvate, metabolite, derivative, or prodrug thereof;
c) identifying or measuring a second brain activity by performing another brain evaluation (e.g., another electroencephalogram (EEG)) on the individual subsequent to administering the first amount of the one or more 5-HT receptor agonist (e.g., psilocybin), or a pharmaceutically acceptable salt, solvate, metabolite, derivative, or prodrug thereof; and
d) administering to the individual a second (e.g., therapeutically effective) amount of one or more 5-HT receptor agonist (e.g., psilocybin), or a pharmaceutically acceptable salt, solvate, metabolite, derivative, or prodrug thereof (e.g., thereby treating brain inflammation (e.g., encephalitis) or brain fog, in the individual).

In some embodiments, the individual is suffering from or susceptible to brain inflammation (e.g., encephalitis) or brain fog.

In some embodiments, the method further comprises subjecting the individual to one or more stimulus.

Provided in some embodiments herein is a method for identifying or measuring a therapeutically effective dose of a 5-hydroxytryptamine (5-HT) receptor agonist (e.g., psilocybin) administered to an individual (e.g., in need thereof), comprising:
a) identifying or measuring a first brain activity of the individual by performing a brain evaluation (e.g., an electroencephalogram (EEG)) on the individual;
b) administering to the individual one or more 5-hydroxytryptamine (5-HT) receptor agonist (e.g., psilocybin), or a pharmaceutically acceptable salt, solvate, metabolite, derivative, or prodrug thereof;
c) subjecting the individual to one or more stimulus (e.g., one or more auditory stimulus or one or more a visual stimulus); and
d) identifying or measuring a second brain activity by performing another brain evaluation (e.g., another electroencephalogram (EEG)) on the individual) subsequent to administering the one or more 5-HT receptor agonist (e.g., psilocybin), or a pharmaceutically acceptable salt, solvate, metabolite, derivative, or prodrug thereof (e.g., wherein a change in brain activity between the first brain activity and the second brain activity indicates that the therapeutically effective dose of the one or more 5-HT agonist has been administered to the individual).

In some embodiments, the individual is subjected to a stimulus before administration of the one or more 5-HT receptor agonist (e.g., psilocybin). In some preferred embodiments, the individual is subjected to a stimulus after administration of the one or more 5-HT receptor agonist (e.g., psilocybin). In some preferred embodiments, the method further comprises identifying or measuring a second brain activity after administering the one or more 5-HT receptor agonist (e.g., psilocybin) and subjecting the individual to a stimulus.

In some embodiments, an increase in brain activity (e.g., in certain regions of the brain) between the first brain activity and the second brain activity indicates that the therapeutically effective dose of the one or more 5-HT agonist has been administered to the individual.

In some embodiments, a decrease in brain activity (e.g., in certain regions of the brain) between the first brain activity and the second brain activity indicates that the therapeutically effective dose of the one or more 5-HT agonist has been administered to the individual.

In some embodiments, the method further comprises administering a therapeutically effective amount of one or more 5-HT receptor agonist (e.g., psilocybin), or a pharmaceutically acceptable salt, solvate, metabolite, derivative, or prodrug thereof, to the individual subsequent to identifying or measuring the second brain activity.

Provided in some embodiments herein is a method for treating a mental, a behavioral, or a neuropsychiatric condition, and/or symptoms thereof, in an individual (e.g., in need thereof), comprising:
a) identifying or measuring a first brain activity of the individual by performing a brain evaluation (e.g., an electroencephalogram (EEG)) on the individual;
b) administering to the individual one or more 5-hydroxytryptamine (5-HT) receptor agonist (e.g., psilocybin), or a pharmaceutically acceptable salt, solvate, metabolite, derivative, or prodrug thereof;
c) subjecting the individual to one or more stimulus (e.g., one or more auditory stimulus or one or more a visual stimulus); and
d) identifying or measuring a second brain activity by performing another brain evaluation (e.g., another electroencephalogram (EEG)) on the individual subsequent to administering the one or more 5-HT receptor agonist (e.g., psilocybin), or a pharmaceutically acceptable salt, solvate, metabolite, derivative, or prodrug thereof (e.g., wherein the one or more 5-HT agonist administered to the individual is a therapeutically effective amount of the one or more 5-HT agonist (e.g., as determined by a change in brain activity between the first brain activity and the second brain activity)).

In some embodiments, the brain evaluation is a brain imaging test. In some embodiments, the brain evaluation is selected from the group consisting of an electroencephalogram (EEG), a magnetoencephalogram (MEG), functional near-infrared spectroscopy (fNIRS), PET, and a transcranial functional ultrasound imaging. In some embodiments, the other brain evaluation is selected from the group consisting of an electroencephalogram (EEG), a magnetoencephalogram (MEG), functional near-infrared spectroscopy (fNIRS), PET, and a transcranial functional ultrasound imaging. In some embodiments, the brain evaluation and the other brain evaluation are the same type of brain test. In some embodiments, the brain evaluation and the other brain evaluation are each a different type of brain test. In some embodiments, the brain evaluation is an EEG. In some embodiments, the other brain evaluation is an EEG. In some embodiments, the brain evaluation is an EEG and the other brain evaluation is an EEG.

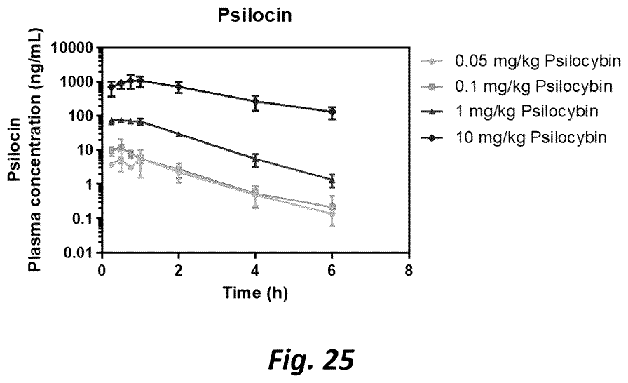

In some embodiments, the PET or fNRIS is used to identify target receptor occupancy in the brain. In some embodiments, the target receptor is one or more 5HT receptor. In some embodiments, the PET or fNRIS is used to identify high and low responding individuals. Identification of high and low responding individuals has been shown in animal studies (FIGS. 24-28). In some embodiments, the PET or fNRIS are used in accordance with the methods disclosed herein to provide personalized care to patients that are either high or low responding individuals.

In some embodiments, the brain evaluation is an initial (e.g., baseline) brain imaging test performed prior to subjecting the individual to the one or more stimulus.

In some embodiments, the one or more stimulus comprises one or more auditory stimulus. In some embodiments, the one or more stimulus is one or more auditory stimulus.

In some embodiments, the one or more auditory stimulus comprises a first auditory stimulus and a second auditory stimulus. In some embodiments, the one or more auditory stimulus is a paired auditory stimuli.

In some embodiments, the first auditory stimulus and the second auditory stimulus are successive.

In some embodiments, the one or more auditory stimulus has a frequency of at least 20 Hertz (Hz) (e.g., 50 or more, 100 Hz or more, 200 Hz or more, 300 Hz or more, 400 Hz or more, 500 Hz or more, 600 Hz or more, 700 Hz or more, 800 Hz or more, 900 Hz or more, 1,000 Hz or more, or 5,000 Hz or more). In some embodiments, the one or more auditory stimulus has a frequency of at most 10,000 Hertz (Hz) (e.g., 10,000 Hz or less, 5,000 Hz or less, 1,000 Hz or less, 900 Hz or less, 800 Hz or less, 700 Hz or less, 600 Hz or less, 500 Hz or less, 400 Hz or less, 300 Hz or less, 200 Hz or less, or 100 Hz or less). In some embodiments, the one or more auditory stimulus has a frequency of 20 Hz to 10,000 Hz. In some embodiments, the one or more auditory stimulus has a frequency of 100 Hz to 1,000 Hz. In some embodiments, the one or more auditory stimulus has a frequency of 300 Hz to 700 Hz. In some embodiments, the one or more auditory stimulus has a frequency of 300 Hz. In some embodiments, the one or more auditory stimulus has a frequency of 700 Hz.

In some embodiments, the first auditory stimulus and the second auditory stimulus have the same acoustic frequency (e.g., 300 Hertz (Hz) and 300 Hz). In some embodiments, the one or more auditory stimulus comprises matched paired tones. In some embodiments, the first auditory stimulus and the second auditory stimulus have a different acoustic frequency (e.g., 300 Hz and 700 Hz). In some embodiments, the one or more auditory stimulus comprises mismatched paired tones.

In some embodiments, the second auditory stimulus is at least 10 milliseconds (ms) (e.g., 20 ms or more, 30 ms or more, 50 ms or more, 100 ms or more, 200 ms or more, 300 ms or more, 400 ms or more, 500 ms or more, 600 ms or more, 700 ms or more, 800 ms or more, 900 ms or more, 1,000 ms or more, 5,000 ms or more, 10,000 ms or more, or 100,000 ms or more) after the first auditory stimulus. In some embodiments, the second auditory stimulus is at most 10,000 ms (e.g., 10,000 ms or less, 5,000 ms or less, 1,000 ms or less, 900 ms or less, 800 ms or less, 700 ms or less, 600 ms or less, 500 ms or less, 400 ms or less, 300 ms or less, 200 ms or less, 100 ms or less, 50 ms or less, or 10 ms or less) after the first auditory stimulus. In some embodiments, the second auditory stimulus is 10,000 ms to 10 ms after the first auditory stimulus. In some embodiments, the second auditory stimulus is 1,000 ms to 100 ms after the first auditory stimulus.

In some embodiments, the time period between the first auditory stimulus and the second auditory stimulus is fixed. In some embodiments, the time period between the first auditory stimulus and the second auditory stimulus is random.

In some embodiments, the one or more auditory stimulus is one or more sustained auditory stimulus.

In some embodiments, the one or more sustained auditory stimulus occurs for at least 1 ms (e.g., 1 ms or more, 10 ms or more, 50 ms or more, 100 ms or more, 200 ms or more, 300 ms or more, 400 ms or more, 500 ms or more, 600 ms or more, 700 ms or more, 800 ms or more, 900 ms or more, 1,000 ms or more, 5,000 ms or more, 10,000 ms or more, or 50,000 ms or more). In some embodiments, the one or more sustained auditory stimulus occurs for at most 60,000 ms (e.g., 50,000 ms or less, 10,000 ms or less, 5,000 ms or less, 1,000 ms or less, 900 ms or less, 800 ms or less, 700 ms or less, 600 ms or less, 500 ms or less, 400 ms or less, 300 ms or less, 200 ms or less, 100 ms or less, 50 ms or less, 10 ms or less, or 1 ms or less). In some embodiments, the one or more sustained auditory stimulus occurs for 1 ms to 60,000 ms. In some embodiments, the one or more sustained auditory stimulus occurs for 100 ms to 1,000 ms. In some embodiments, the one or more sustained auditory stimulus occurs for 500 ms.

In some embodiments, the one or more sustained auditory stimulus has a fixed acoustic frequency (e.g., occurring for a period of time of greater than or equal to about five hundred ms).

In some embodiments, the one or more sustained auditory stimulus has a variable acoustic frequency (e.g., occurring for a period of time of greater than or equal to about five hundred ms).

In some embodiments, the one or more stimulus comprises one or more visual stimulus. In some embodiments, the one or more stimulus is one or more visual stimulus.

In some embodiments, the one or more visual stimulus is an image, a series of images, a movie (e.g., a .GIF), or a contrast. In some embodiments, the one or more visual stimulus is a shape, a pattern, an emotional face, a color, or the like. In some embodiments, the one or more visual stimulus is a series of shapes, a series of patterns, a series of emotional faces, or the like. In some embodiments, the one or more visual stimulus is a flash. In some embodiments, the one or more visual stimulus is a video.

In some embodiments, the one or more visual stimulus comprises a first visual stimulus and a second visual stimulus. In some embodiments, the one or more visual stimulus is a paired visual stimuli.

In some embodiments, the first visual stimulus and the second visual stimulus are successive.

In some embodiments, the one or more visual stimulus occurs for at least 10 milliseconds (ms) (e.g., 20 ms or more, 30 ms or more, 50 ms or more, 100 ms or more, 200 ms or more, 300 ms or more, 400 ms or more, 500 ms or more, 600 ms or more, 700 ms or more, 800 ms or more, 900 ms or more, 1 second (s) or more, 5 s or more, 10 s or more, 30 s or more, 1 minute (min) or more, 5 mins or more, 10 mins or more, 20 mins or more, 30 mins or more, or 1 hour or more). In some embodiments, the one or more visual stimulus occurs for at most 1 hour (e.g., 30 min or less, 20 min or less, 10 min or less, 5 min or less, 1 min or less, 50 s or less, 40 s or less, 30 s or less, 20 s or less, 10 s or less, 5 s or less, 1 s or less, 900 ms or less, 800 ms or less, 700 ms or less, 600 ms or less, 500 ms or less, 400 ms or less, 300 ms or less, 200 ms or less, 100 ms or less, 50 ms or less, 20 ms or less, or 10 ms or less). In some embodiments, the one or more visual stimulus occurs for 1 hour to 10 ms. In some embodiments, the one or more visual stimulus occurs for 10 ms to 1 s. In some embodiments, the one or more visual stimulus occurs for 1 min to 10 mins.

In some embodiments, the second visual stimulus is at least 10 milliseconds (ms) (e.g., 20 ms or more, 30 ms or more, 50 ms or more, 100 ms or more, 200 ms or more, 300 ms or more, 400 ms or more, 500 ms or more, 600 ms or more, 700 ms or more, 800 ms or more, 900 ms or more, 1,000 ms or more, 5,000 ms or more, 10,000 ms or more, or 100,000 ms or more) after the first visual stimulus. In some embodiments, the second visual stimulus is at most 10,000 ms (e.g., 10,000 ms or less, 5,000 ms or less, 1,000 ms or less, 900 ms or less, 800 ms or less, 700 ms or less, 600 ms or less, 500 ms or less, 400 ms or less, 300 ms or less, 200 ms or less, 100 ms or less, 50 ms or less, or 10 ms or less) after the first visual stimulus. In some embodiments, the second visual stimulus is 10,000 ms to 10 ms after the first visual stimulus. In some embodiments, the second visual stimulus is 1,000 ms to 100 ms after the first visual stimulus.

In some embodiments, the time period between the first visual stimulus and the second visual stimulus is fixed. In some embodiments, the time period between the first auditory stimulus and the second auditory stimulus is random.

In some embodiments, the one or more visual stimulus is one or more sustained visual stimulus.

In some embodiments, the one or more sustained auditory stimulus occurs for at least 1 ms (e.g., 1 ms or more, 10 ms or more, 50 ms or more, 100 ms or more, 200 ms or more, 300 ms or more, 400 ms or more, 500 ms or more, 600 ms or more, 700 ms or more, 800 ms or more, 900 ms or more, 1,000 ms or more, 5,000 ms or more, 10,000 ms or more, or 50,000 ms or more). In some embodiments, the one or more sustained auditory stimulus occurs for at most 60,000 ms (e.g., 50,000 ms or less, 10,000 ms or less, 5,000 ms or less, 1,000 ms or less, 900 ms or less, 800 ms or less, 700 ms or less, 600 ms or less, 500 ms or less, 400 ms or less, 300 ms or less, 200 ms or less, 100 ms or less, 50 ms or less, 10 ms or less, or 1 ms or less). In some embodiments, the one or more sustained auditory stimulus occurs for 1 ms to 60,000 ms. In some embodiments, the one or more sustained auditory stimulus occurs for 100 ms to 1,000 ms. In some embodiments, the one or more sustained auditory stimulus occurs for 500 ms.

In some embodiments, the first visual stimulus and the second visual auditory stimulus comprise shapes, patterns, emotional faces, and/or colors, and have the same contrast.

In some embodiments, the first visual stimulus and the second visual auditory stimulus comprise shapes, patterns, emotional faces, and/or colors, and have a different contrast.

In some embodiments, the individual is subjected to the one or more brain imaging test for at least once per day (e.g., twice per day or more, thrice per day or more, and so on). In some embodiments, the individual is subjected to the one or more brain imaging test for at least 1 s per day (e.g., 1 s per day or more, 10 s per day or more, 30 s per day or more, 1 min per day or more, 5 min per day or more, 10 min per day or more, or 30 min per day or more). In some embodiments, the individual is subjected to the one or more brain imaging test for 30 s per day to 30 mins per day. In some embodiments, the individual is subjected to the one or more brain imaging test for 1 min per day to 10 mins per day.

In some embodiments, the individual is subjected to the one or more brain imaging test at least one day (e.g., two days, three days, four days, five days, six days, or seven days) per week (e.g., for at least one week (e.g., for one week or more, two weeks or more, four weeks or more, and so on)).

In some embodiments, the brain evaluation is an initial (e.g., baseline) brain imaging test performed prior to administering the one or more 5-HT receptor agonist (e.g., psilocybin) to the individual.

In some embodiments, the first amount of the one or more 5-HT receptor agonist is administered to the individual subsequent to identifying or measuring the first brain activity of the individual.

In some embodiments, the first amount of the one or more 5-HT receptor agonist (e.g., psilocybin), or a pharmaceutically acceptable salt, solvate, metabolite, derivative, or prodrug thereof, is an initial dose (e.g., that is not a therapeutically effective dose (e.g., that does not provide a sustained or a therapeutic effect to the individual)) of the one or more 5-HT receptor agonist (e.g., psilocybin), or a pharmaceutically acceptable salt, solvate, metabolite, derivative, or prodrug thereof. In some embodiments, the first amount of the one or more 5-HT receptor agonist (e.g., psilocybin), or a pharmaceutically acceptable salt, solvate, metabolite, derivative, or prodrug thereof, is not a therapeutically effective amount of the one or more 5-HT receptor agonist (e.g., psilocybin), or a pharmaceutically acceptable salt, solvate, metabolite, derivative, or prodrug thereof. In some embodiments, the first amount of the one or more 5-HT receptor agonist (e.g., psilocybin), or a pharmaceutically acceptable salt, solvate, metabolite, derivative, or prodrug thereof, produces a significant change in brain activity of the individual. In some embodiments, the first amount of the one or more 5-HT receptor agonist (e.g., psilocybin), or a pharmaceutically acceptable salt, solvate, metabolite, derivative, or prodrug thereof, produces a significant change in brain activity of the individual but does not produce a sustained effect or a therapeutic effect in the individual. In some embodiments, the first amount of the one or more 5-HT receptor agonist (e.g., psilocybin), or a pharmaceutically acceptable salt, solvate, metabolite, derivative, or prodrug thereof, is a therapeutically effective amount of the one or more 5-HT receptor agonist (e.g., psilocybin), or a pharmaceutically acceptable salt, solvate, metabolite, derivative, or prodrug thereof.

In some embodiments, the second brain activity of the individual is identified or measured subsequent to identifying or measuring the first brain activity of the individual. In some embodiments, the second brain activity of the individual is identified or measured subsequent to identifying or measuring the first brain activity of the individual and administering the first amount of the one or more 5-HT receptor agonist (e.g., psilocybin), or a pharmaceutically acceptable salt, solvate, metabolite, derivative, or prodrug thereof.

In some embodiments, the first amount of the one or more 5-HT receptor agonist (e.g., psilocybin) is not sufficient to elicit a significant change in brain activity (e.g., from the first brain activity measurement to the second brain activity measurement) of the individual. In some embodiments, the first amount of the one or more 5-HT receptor agonist (e.g., psilocybin) is sufficient to elicit a measurable (e.g., significant) change in brain activity (e.g., the change in brain activity being from the first brain activity measurement to the second brain activity measurement) of the individual.

In some embodiments, the second amount of the one or more 5-HT receptor agonist is the same 5-HT receptor agonist as the first amount of the one or more 5-HT receptor agonist. In some embodiments, the second amount of the one or more 5-HT receptor agonist is a different 5-HT receptor agonist as the first amount of the one or more 5-HT receptor agonist. In some embodiments, the first amount of the one or more 5-HT receptor agonist is psilocybin and the second amount of the one or more 5-HT receptor agonist is psilocybin.

In some embodiments, the second amount of the one or more 5-HT receptor agonist is administered subsequent to identifying or measuring the first brain activity of the individual. In some embodiments, the second amount of the one or more 5-HT receptor agonist is administered subsequent to administering to the individual the first amount of the one or more 5-HT receptor agonist (e.g., psilocybin), or a pharmaceutically acceptable salt, solvate, metabolite, derivative, or prodrug thereof. In some embodiments, the second amount of the one or more 5-HT receptor agonist is administered subsequent to identifying or measuring the second brain activity. In some embodiments, the second amount of the one or more 5-HT receptor agonist is administered subsequent to identifying or measuring the first brain activity of the individual and subsequent to administering to the individual the first amount of the one or more 5-HT receptor agonist (e.g., psilocybin), or a pharmaceutically acceptable salt, solvate, metabolite, derivative, or prodrug thereof. In some embodiments, the second amount of the one or more 5-HT receptor agonist is administered subsequent to identifying or measuring the first brain activity of the individual and subsequent to identifying or measuring the second brain activity.

In some embodiments, the second amount of the one or more 5-HT receptor agonist (e.g., psilocybin) is not sufficient to maintain a significant change in brain activity (e.g., from the first brain activity measurement to the second brain activity measurement) of the individual. In some embodiments, the second amount of the one or more 5-HT receptor agonist (e.g., psilocybin) is sufficient to maintain a measurable (e.g., significant) change in brain activity (e.g., the change in brain activity being from the first brain activity measurement to the second brain activity measurement) of the individual.

In some embodiments, the second amount of the one or more 5-HT receptor agonist (e.g., psilocybin), or a pharmaceutically acceptable salt, solvate, metabolite, derivative, or prodrug thereof, produces a significant change in brain activity of the individual. In some embodiments, the second amount of the one or more 5-HT receptor agonist (e.g., psilocybin), or a pharmaceutically acceptable salt, solvate, metabolite, derivative, or prodrug thereof, produces a significant change in brain activity of the individual but does not produce a sustained effect in the individual. In some embodiments, the second amount of the one or more 5-HT receptor agonist (e.g., psilocybin), or a pharmaceutically acceptable salt, solvate, metabolite, derivative, or prodrug thereof, is a therapeutically effective amount of the one or more 5-HT receptor agonist (e.g., psilocybin), or a pharmaceutically acceptable salt, solvate, metabolite, derivative, or prodrug thereof.

In some embodiments, the first amount of the one or more 5-HT receptor agonist (e.g., psilocybin) is the same amount as the second amount of the one or more 5-HT receptor agonist (e.g., psilocybin).

In some embodiments, the first amount of the one or more 5-HT receptor agonist (e.g., psilocybin) is a different amount than the second amount of the one or more 5-HT receptor agonist (e.g., psilocybin). In some embodiments, the first amount of the one or more 5-HT receptor agonist (e.g., psilocybin) is a smaller amount than the second amount of the one or more 5-HT receptor agonist (e.g., psilocybin). In some embodiments, the first amount of the one or more 5-HT receptor agonist (e.g., psilocybin) is a larger amount than the second amount of the one or more 5-HT receptor agonist (e.g., psilocybin).

In some embodiments, there is a significant change in brain activity (e.g., from the first brain activity measurement and the second brain activity measurement) of the individual, and the individual is administered the second amount of the one or more 5-HT receptor agonist, the second amount being the same dose or a lower dose than the first amount of the one or more 5-HT receptor agonist.

In some embodiments, there is a lack of brain activity (e.g., compared to the first brain activity measurement). In some embodiments, the lack of brain activity is a resting state brain activity. In some embodiments, the resting state brain activity is measured across two conditions. In some embodiments, the two conditions are eyes open and eyes closed.

In some embodiments, the resting state brain activity is measured for at least 1 second (e.g., 1 s or more, 10 s or more, 20 s or more, 30 s or more, 40 s or more, 50 s or more, 1 min or more, 5 min or more, or 10 min or more). In some embodiments, the resting state brain activity is measured for at most 10 minutes (e.g., 10 min or less, 5 min or less, 1 min or less, 50 s or less, 40 s or less, 30 s or less, 20 s or less, 10 s or less, or 1 s or less). In some embodiments, the resting state brain activity is measured for 1 s to 10 min. In some embodiments, the resting state brain activity is measured for 1 s to 1 min. In some embodiments, the resting state brain activity is measured for 30 s.

In some embodiments, the resting state brain activity is measured daily, twice daily, thrice daily, every other day, three times per week, at least once per week, or the like. In some embodiments, the resting state brain activity is measured for one day, for one week, for one month, for one year, or the like.

In some embodiments, the resting state brain activity is measured while the one or more 5-Ht agonist is administered to the individual. In some embodiments, the resting state brain activity is measured after the one or more 5-HT agonist is administered to the individual.

In some embodiments, there is a lack of a significant change in brain activity (e.g., from the first brain activity measurement and the second brain activity measurement) of the individual, and the individual is administered the second amount of the one or more 5-HT receptor agonist, the second amount being the same or a higher dose than the first amount of the one or more 5-HT receptor agonist.

In some embodiments, the second amount of one or more 5-HT receptor agonist (e.g., psilocybin), or the pharmaceutically acceptable salt, solvate, metabolite, derivative, or prodrug thereof, is administered to the individual subsequent to identifying or measuring a significant change in brain activity of the individual. In some embodiments, the significant change in brain activity of the individual occurs subsequent to a first brain activity measurement and a second brain activity measurement. In some embodiments, the significant change in brain activity of the individual occurs between a first brain activity measurement and a second brain activity measurement (e.g., the change being from a baseline brain activity and the first brain activity measurement).

In some embodiments, the second amount of one or more 5-HT receptor agonist (e.g., psilocybin), or the pharmaceutically acceptable salt, solvate, metabolite, derivative, or prodrug thereof, is administered to the individual subsequent to identifying or measuring a lack of a significant change in brain activity of the individual. In some embodiments, the lack of the significant change in brain activity of the individual occurs subsequent to a first brain activity measurement and a second brain activity measurement. In some embodiments, the lack of the significant change in brain activity of the individual occurs between a first brain activity measurement and a second brain activity measurement (e.g., the change being from a baseline brain activity and the first brain activity measurement).

In some embodiments, a change in the resting state brain activity (e.g., of the prefrontal cortex of the individual) indicates that the therapeutically effective dose of the one or more 5-HT agonist has been administered to the individual.

In some embodiments, the second amount of the one or more 5-HT receptor agonist (e.g., psilocybin) is administered at least 10 minutes (e.g., ten minutes or more, twenty minutes or more, thirty minutes or more, forty minutes or more, fifty minutes or more, one hour or more, two hours or more, three hours or more, four hours or more, or the like) after the first amount of the one or more 5-HT receptor agonist (e.g., psilocybin). In some embodiments, the first amount of the one or more 5-HT receptor agonist (e.g., psilocybin) and the second amount of the one or more 5-HT receptor agonist (e.g., psilocybin) are administered within a 24 hour period (e.g., within a third minute period, within an hour period, within a 4 hour period, within a 6 hour period, within an 8 hour period, within a 12 hour period, or the like) of each other. In some embodiments, the first amount of the one or more 5-HT receptor agonist (e.g., psilocybin) and the second amount of the one or more 5-HT receptor agonist (e.g., psilocybin) are administered on or after a 24 hour period (e.g., the second amount is administered more than or equal to 24 hours after the first amount, the second amount is administered more than or equal to 48 hours after the first amount, or the like).

In some embodiments, the second brain activity is identified or measured at least five minutes (e.g., five minutes or more, ten minutes or more, twenty minutes or more, thirty minutes or more, forty minutes or more, fifty minutes or more, one hour or more, two hours or more, three hours or more, four hours or more, or the like) after the first brain activity is identified or measured. In some embodiments, the first brain activity and the second brain activity are identified or measured within a 24 hour period (e.g., within a 5 minute period, within a 10 minute period, within a thirty minute period, within a 1 hour period, within a 4 hour period, within a 6 hour period, within an 8 hour period, within a 12 hour period, or the like) of each other. In some embodiments, the first brain activity and the second brain activity are measured or identified on or after a 24 hour period (e.g., the second brain activity is measured or identified more than or equal to 24 hours after the first brain activity, the second brain activity is measured or identified more than or equal to 48 hours after the first brain activity, or the like).

In some embodiments, the brain activity or the lack thereof is measured in a time domain (e.g., as time-locked changes) to a paired auditory stimuli as a voltage (e.g., evoked potentials), a magnetic field, or a hemodynamic signal.

In some embodiments, the brain activity or the lack thereof is measured in a frequency domain as a voltage, a magnetic field, or a hemodynamic signal.

In some embodiments, the hemodynamic signal is a flux, a flow, a velocity, or an oxygenation level.

In some embodiments, the voltage is power in alpha bands, beta bands, gamma bands, delta bands, or theta bands.

The Delta band can represent low frequency brain activity associated with resting states, deep concentration, or sleep. When activity is high (warmer colors) in this band, the brain is in a resting or drowsy state. When activity in this band is low (cooler colors) then the brain is more active and alert.

The Theta band can be associated with a relaxed state of mind that is most active when one is conscious, but not engaging in learning or a specific task. For example, theta brain wave activity is higher when attention is inwardly focused like during meditation. When the brain is actively engaged in learning, problem solving, or outwardly focused then theta activity is lower.

The Alpha band can be associated with an active level of thought during which one is not actively processing information. Alpha consists of the everyday thoughts that flow through the mind when not concentrating. It is considered a learning state when one are alert but not actively processing.

The Beta band can be associated with active thought and information processing when one is focused on a task or outcome. High levels of Beta can reflect a busy mind or actively engaged brain state, whereas lower levels of Beta can reflect a calmer mind.

The Gamma band can reflect a deeper focused brain state where attention is locked on a task, deep concentration, or problem solving. High gamma activity can also reflect synchronous processing of multiple streams of information in the brain. Lower levels of gamma activity can reflect that the brain is not locked into working on a specific task or solving a specific problem at that moment.

In some embodiments, the second amount of the one or more 5-HT receptor agonist (e.g., psilocybin) is not sufficient to elicit a significant change in brain activity (e.g., from the second brain activity measurement to another brain activity measurement (e.g., the first brain activity measurement, a third brain activity measurement, a fourth brain activity measurement, etc.)) of the individual. In some embodiments, the second amount of the one or more 5-HT receptor agonist (e.g., psilocybin) is sufficient to maintain a measurable (e.g., from the second brain activity measurement to another brain activity measurement (e.g., the first brain activity measurement, a third brain activity measurement, a fourth brain activity measurement, etc.)) of the individual.

In some embodiments, the other brain activity measurement is the first brain activity measurement, a third brain activity measurement, a fourth brain activity measurement, or so on.

In some embodiments, the method further comprises identifying or measuring another brain activity by performing a third (e.g., a fourth, a fifth, a six, or more) brain evaluation (e.g., a third (e.g., a fourth, a fifth, a six, or more) electroencephalogram (EEG)) on the individual subsequent to administering the second amount (e.g., a third amount, a fourth amount, a fifth amount, or more) of the one or more 5-HT receptor agonist (e.g., psilocybin), or a pharmaceutically acceptable salt, solvate, metabolite, derivative, or prodrug thereof.

In some embodiments, a third (e.g., therapeutically effective) amount of one or more 5-HT receptor agonist (e.g., psilocybin) is administered to the individual. In some embodiments, the third amount is administered between the first and second amount as in intervening dose. In some embodiments, the third amount is administered after the second amount as a subsequent dose.

In some embodiments, the therapeutically effective amount of 5-HT receptor agonist or a pharmaceutically acceptable salt, solvate, metabolite, derivative, or prodrug thereof administered to the individual is determined based on the (e.g., change or lack of change in brain activity determined by a) brain evaluation of the individual. For example, in some instances, subsequent to measuring a significant change in brain activity (e.g., between a first brain activity measurement and a second brain activity measurement), the second amount of one or more 5-HT receptor agonist (e.g., psilocybin), or the pharmaceutically acceptable salt, solvate, metabolite, derivative, or prodrug thereof, is administered to the individual at the same dose or a lower dose than the first amount. In other instances, subsequent to measuring a lack of significant change in brain activity (e.g., between a first brain activity measurement and a second brain activity measurement), the second amount of one or more 5-HT receptor agonist (e.g., psilocybin), or the pharmaceutically acceptable salt, solvate, metabolite, derivative, or prodrug thereof, is administered to the individual at a higher dose than the first amount.

In some embodiments, a (e.g., maintenance) dose of the one or more 5-HT receptor agonist (e.g., psilocybin) is administered to the individual subsequent to each identification or measurement of a brain activity (e.g., obtained subsequent to performing a brain evaluation) of the individual. In some embodiments, it takes several doses (e.g., two or more, three to more, five or more, or six or more) and measurements (e.g., changes in brain activity (e.g., two or more, three or more, four or more, five or more, or six or more)) to establish a therapeutic dose of the one or more 5-HT receptor agonist (e.g., psilocybin). In some embodiments, the one or more 5-HT receptor agonist (e.g., psilocybin) is titrated in the individual until the therapeutic dose is established.

In some embodiments, the method further comprises administering to the individual an (e.g., therapeutically effective) amount of one or more 5-HT receptor agonist, or a pharmaceutically acceptable salt, solvate, metabolite, derivative, or prodrug thereof a third time, wherein the third time is a time between the first time and the second time or after the second time.

In some embodiments, the method further comprises administering to the individual an (e.g., therapeutically effective) amount of one or more 5-HT receptor agonist, or a pharmaceutically acceptable salt, solvate, metabolite, derivative, or prodrug thereof a fourth time. In some embodiments, the fourth time is a time between the first and third time. In some embodiments, the fourth time is a time between the first and second time. In some embodiments, the fourth time is a time after the first time. In some embodiments, the fourth time is a time after the second time. In some embodiments, the fourth time is a time after the third time.

In some embodiments, the method further comprises performing one or more heart test (e.g., to determine heart rate or cardiovascular activity) on the individual. In some embodiments, the heart test is performed for an amount of time sufficient to determine the cardiovascular activity (e.g., heart rate) of the individual (e.g., for at least 30 seconds).

In some embodiments, the method further comprises performing one or more test to measure skin conductance or impedance.

In some embodiments, the method further comprises integrating electronic patient reported outcomes or subjective mood, affect, coping, sleep quality, stress, anxiety, memory, and/or other emotional or functional data with brain imaging or brain activity levels.

In some embodiments, the method further comprises providing data (e.g., patterns, clusters, classifications, amplitudes, frequencies, or magnitude) on a device (e.g., a phone, tablet, or computer) to the individual and/or a caregiver (e.g., physicians, counselors, psychologist, or spiritual leader). In some embodiments, the data comprises visual representations of brain activity data and/or survey or patient reported outcomes (e.g., to help convey the effectiveness or lack thereof of the administration of a 5-HT receptor agonist provided herein).

In some embodiments, the (e.g., one or more) 5-HT receptor agonist is a 5-HT2 receptor agonist. In some embodiments, the 5-HT2 receptor agonist is a 5-HT_{2A} receptor agonist, a 5-HT2B receptor agonist and/or a 5-HT2C receptor agonist. In some embodiments, the (e.g., one or more) 5-HT receptor agonist is psilocin or psilocybin or a pharmaceutically acceptable salt, solvate, metabolite, derivative, or prodrug thereof. In some embodiments, the therapeutically effective amount of the 5-HT receptor agonist or a pharmaceutically acceptable salt, solvate, metabolite, derivative, or prodrug thereof is provided to the subject in need thereof in an amount insufficient to provide a hallucinogenic experience.

In some embodiments, the one or more 5-HT receptor agonist, or a pharmaceutically acceptable salt, solvate, metabolite, derivative, or prodrug thereof is a 5HT2A receptor agonist.

In some embodiments, the one or more 5-HT receptor agonist is psilocybin or psilocin, or a pharmaceutically acceptable salt, solvate, metabolite, derivative, or prodrug thereof. In some embodiments, the one or more 5-HT receptor agonist is an active form of a 5-HT receptor agonist provided herein. In some embodiments, the active form of the 5-HT receptor agonist is psilocin.

In some embodiments, the therapeutically effective amount of the 5HT receptor agonist or a pharmaceutically acceptable salt, solvate, metabolite, derivative, or prodrug thereof is provided to a subject in need thereof in an amount and/or formulation to provide a maximum plasma concentration (Cₘₐₓ) of (e.g. active form of the) 5HT receptor agonist or a pharmaceutically acceptable salt, solvate, metabolite, derivative, or prodrug thereof of at least 0.001 ng/mL (e.g., 0.001 ng/mL or more, 0.01 ng/mL or more, 0.1 ng/mL or more, ng/mL or more, 10 ng/mL or more, or 100 ng/mL or more). In some embodiments, the therapeutically effective amount of the 5HT receptor agonist or a pharmaceutically acceptable salt, solvate, metabolite, derivative, or prodrug thereof is provided to a subject in need thereof in an amount and/or formulation to provide a maximum plasma concentration (Cₘₐₓ) of (e.g. active form of the) 5HT receptor agonist or a pharmaceutically acceptable salt, solvate, metabolite, derivative, or prodrug thereof of at most 100 ng/mL (e.g., 100 ng/mL or less, 10 ng/mL or less, 1 ng/mL or less, 0.1 ng/mL or less, 0.01 ng/mL or less, or 0.001 ng/mL or less). In some embodiments, the therapeutically effective amount of the 5HT receptor agonist or a pharmaceutically acceptable salt, solvate, metabolite, derivative, or prodrug thereof is provided to a subject in need thereof in an amount and/or formulation to provide a maximum plasma concentration (Cₘₐₓ) of (e.g. active form of the) 5HT receptor agonist or a pharmaceutically acceptable salt, solvate, metabolite, derivative, or prodrug thereof of about 0.001 ng/mL to about 100 ng/mL (e.g., about 0.01 ng/mL to about 100 ng/mL, about 0.01 ng/mL to about 50 ng/mL, or about 0.1 ng/mL to about 50 ng/mL).

In some embodiments, the Cₘₐₓ is a therapeutically effective concentration. In some embodiments, the Cₘₐₓ is not a therapeutically effective concentration (e.g., before an optimal dose is determined by the analysis of a brain evaluation of the individual).

In some embodiments, the method comprises administering to the individual the therapeutically effective amount of one or more 5-HT receptor agonist, or a pharmaceutically acceptable salt, solvate, metabolite, derivative, or prodrug thereof, active 5-HT receptor agonist at a dose sufficient to provide a maximum plasma concentration (Cₘₐₓ) of the active 5-HT receptor agonist below the hallucinogenic effective threshold of the active 5-HT receptor agonist and a minimum plasma concentration (Cₘᵢₙ) of the active 5-HT receptor agonist of at least the therapeutically effective threshold of the active 5-HT receptor agonist in the individual. In some embodiments, the level of the active 5-HT receptor agonist is maintained in the individual (e.g., in a biological sample (e.g., serum, plasma, or whole blood) of the individual) above the therapeutically effective threshold and below the hallucinogenic effective threshold of the active 5-HT receptor agonist.

In some embodiments, the therapeutically effective amount of the one or more 5-HT receptor agonist, or a pharmaceutically acceptable salt, solvate, metabolite, derivative, or prodrug thereof, is administered to the individual in need thereof in an amount and/or formulation to provide a plasma concentration of the active 5-HT receptor agonist of at least 0.1 ng/mL (e.g., at least 0.2 ng/mL, at least 0.3 ng/mL, at least 0.5 ng/mL, or the like) in the individual for at least 2 hours (e.g., at least 12 hours, at least 24 hours, at least 36 hours, at least 48 hours, at least 72 hours, at least 96 hours, at least 120 hours, at least 144 hours, or the like).

In some embodiments, the therapeutically effective amount of the one or more 5-HT receptor agonist, or a pharmaceutically acceptable salt, solvate, metabolite, derivative, or prodrug thereof, is administered to the individual in need thereof over an extended period of time (e.g., daily for a week or more, twice per day for a week or more, three times per day for a week or more, every other day for a week or more, two times a week, once a week, bi-weekly for a month or more, or the like).

In some embodiments, the one or more 5-HT receptor agonist, or the pharmaceutically acceptable salt, solvate, metabolite, derivative, or prodrug thereof, is a hallucinogenic compound (e.g., wherein the hallucinogenic compound produces a hallucinogenic effect (e.g., an adverse event, a clinically important effect (e.g., clinically important impairment of the individual, altered (e.g., visual, auditory, body, time and space) perception, altered cognition, impaired attention, drowsiness, and/or confusion)) in the individual in need thereof at or above the hallucinogenic threshold (e.g., at or above a Cₘₐₓ above the hallucinogenic effective threshold)).

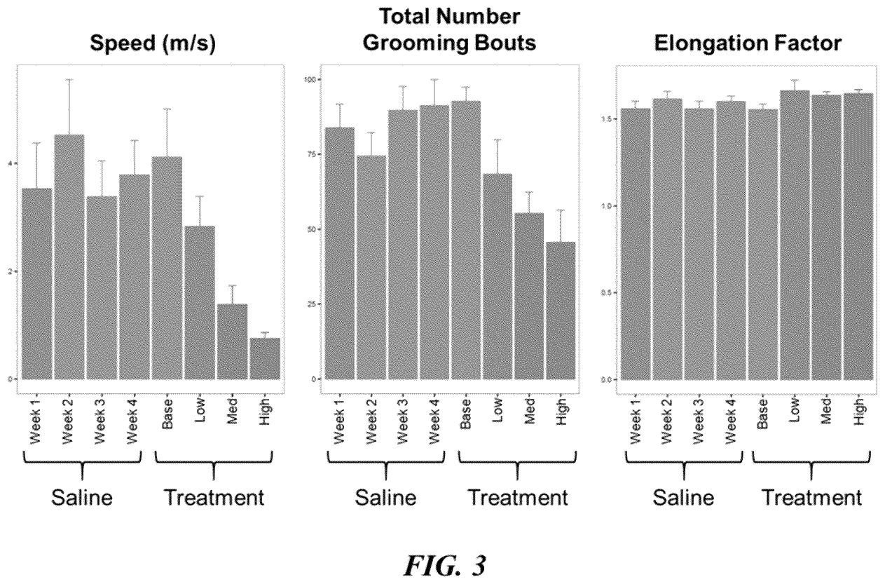

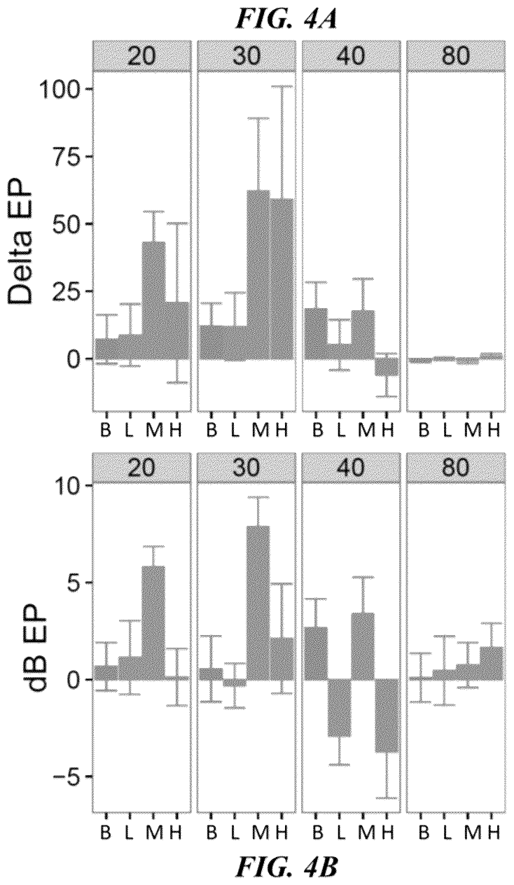

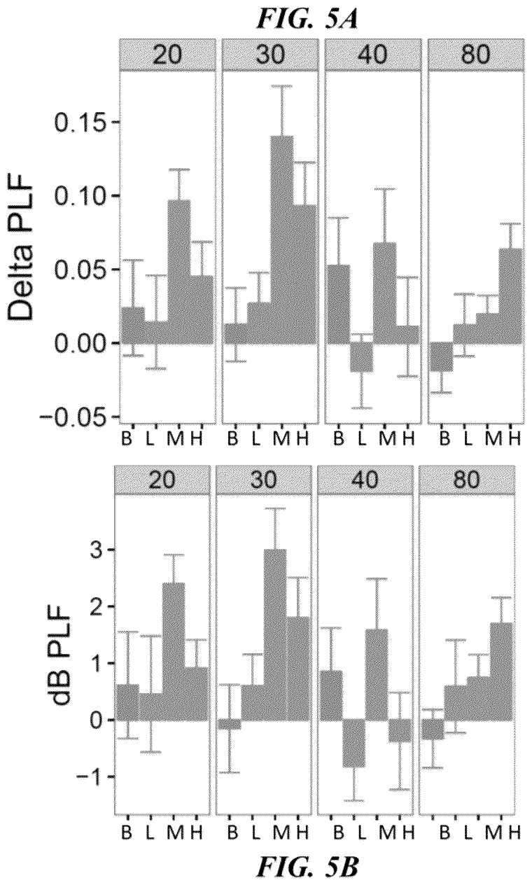

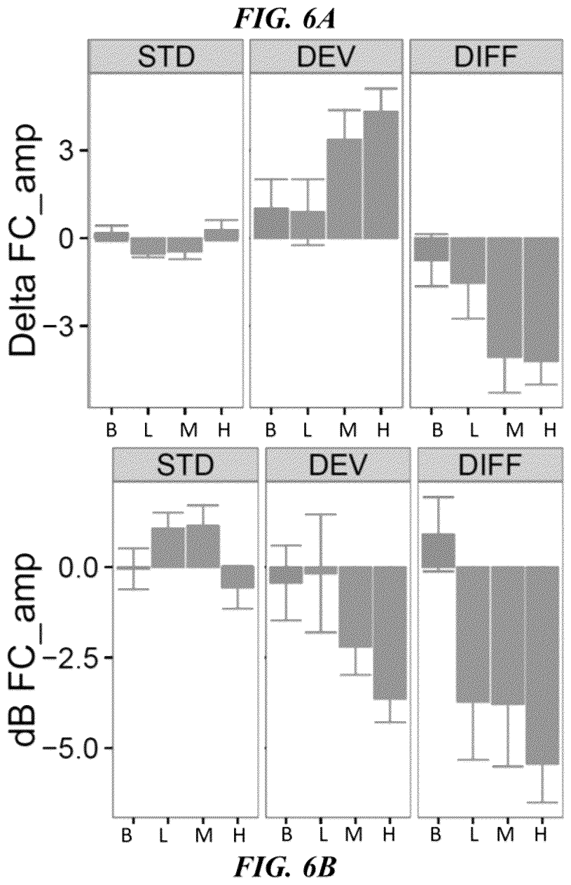

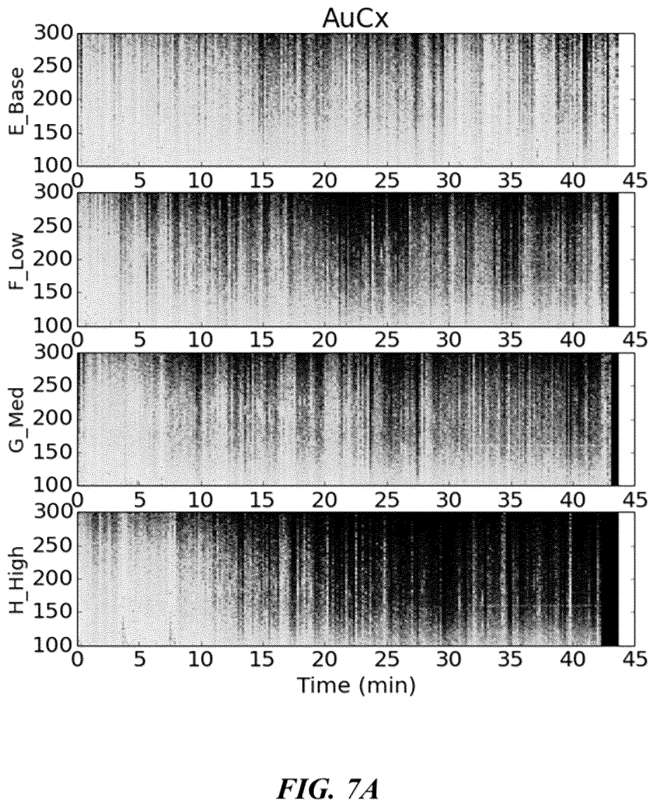

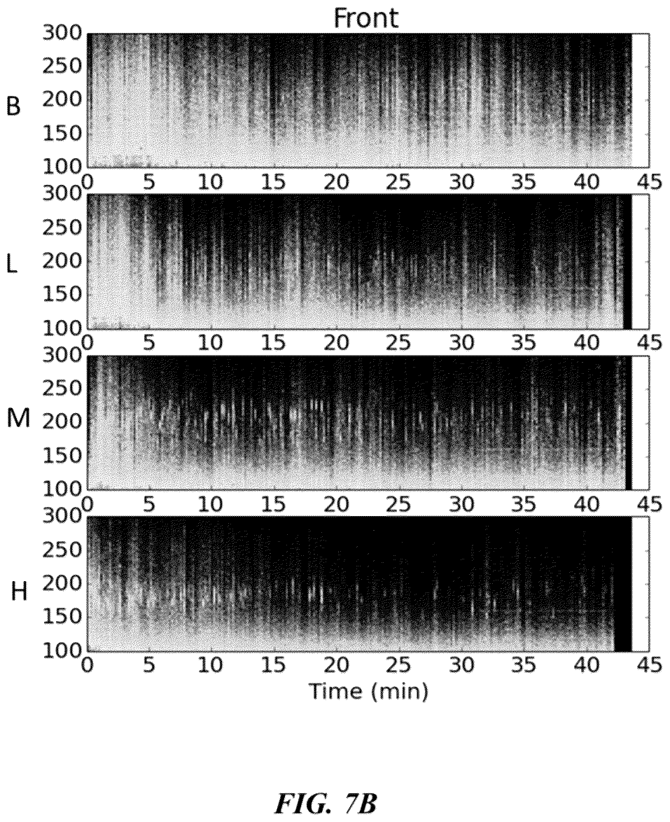

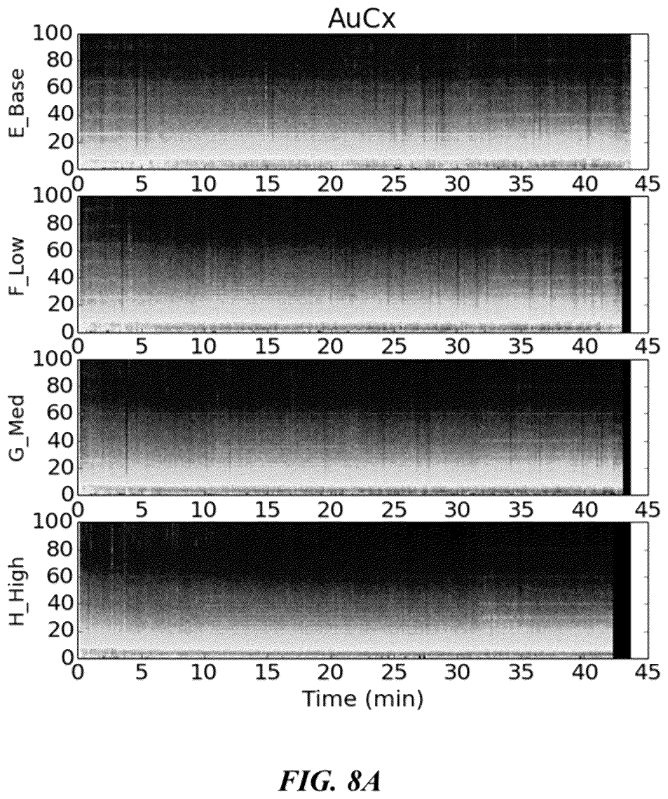

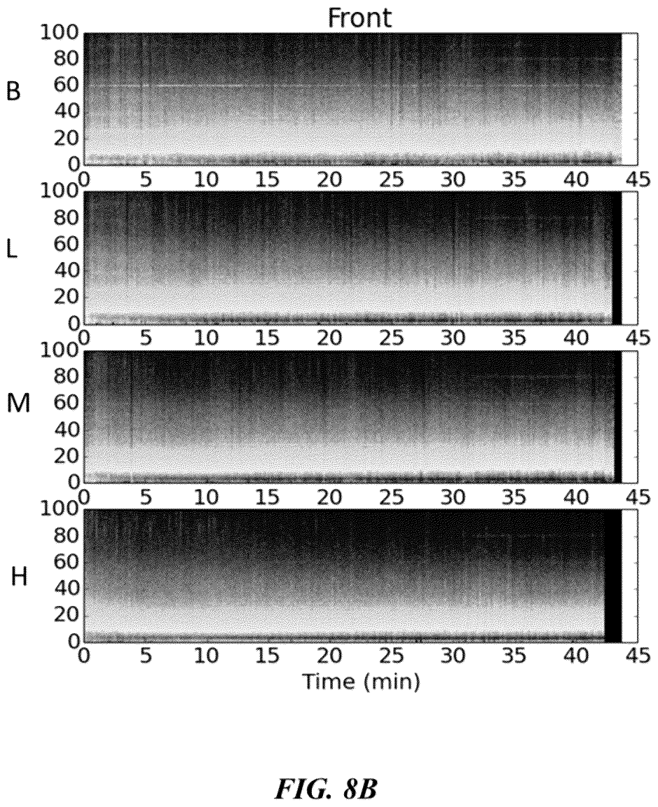

In some instances, behavioral responses observed subsequent to a brain evaluation corresponds well with (and suggest validation of) the electroencephalogram (EEG) results provided herein (e.g., see FIG. 3). In some instances, brain activity is determined from a difference between a standard tone waveform and a deviant tone waveform. In some instances, a larger difference between the standard tone waveform and the deviant form waveform suggests an asynchronization (e.g., a stabilization and/or a normalization) in brain activity. In some instances, a higher dose (e.g., than another (e.g., a first) dose) of psilocybin provides an asynchronization (e.g., a stabilization and/or a normalization) in brain activity in the individual (FIG. 6A and FIG. 6B). In some instances, a therapeutically effective dose (e.g., an optimal dose, a maintenance dose, an initial dose, or the like) of a compound provided herein (e.g., psilocybin) is determined based on a difference between a standard tone waveform and a deviant form waveform. In some instances, brain activity is determined from a difference between a control evoked power recording or a phase locking factor recording and a compound evoked power recording or a phase locking factor recording (e.g., at each frequency (20 Hz, 30 Hz, 40 Hz, and 8 Hz) for control (saline) and compound (psilocybin)). In some instances, the change in the difference between the control and compound recording across a particular frequency indicates the dose at which brain activity becomes asynchronized, for example, which indicates the therapeutically effective dose has been administered to the individual (FIG. 4A, FIG. 4B, FIG. 5A, and FIG. 5B). In some instances, brain activity is determined from an ongoing spectral power recording and calculation (e.g., as a function of time over each daily session before and between stimulation periods). In some instances, for example, as shown in FIG. 7A, FIG. 7B, FIG. 8A, and FIG. 8B, the darker regions of the ongoing spectral recording are indicative of a decrease in general brain activity, for example which suggests a stabilization of general brain activity. In some instances, a higher dose corresponds with a decrease in general brain activity (which are darker regions), such as, indicating a loss in spectral power, such as, suggesting brain activity asynchronization (e.g., normalization of brain function). In some instances, any one of or a combination of the methods provided herein are used to determine a therapeutically effective dose (e.g., an optimal dose, a maintenance dose, an initial dose, or the like) to be administered to or administered to an individual (e.g., in need thereof). In some instances, any one of or a combination of the methods provided herein are used to titrate the dose of a compound provided herein (e.g., psilocybin) in an individual (e.g., in need thereof).

Provided herein are methods for managing or treating disorders or conditions, or treating symptoms of disorders or conditions, in an individual (e.g., in need thereof), comprising administering one or more 5-HT receptor agonists, or pharmaceutically acceptable salts, solvates, metabolites, derivatives, or prodrugs thereof.

In some embodiments, the individual is in need of a treatment for the brain disorder or condition. In some embodiments, the individual is in need of a treatment or a reduction in the incidence of the mental, the behavioral, or the neuropsychiatric condition, and/or the symptoms thereof. In some embodiments, the individual is susceptible to or suffering from a brain disorder or condition (e.g., a mental condition, a behavioral condition, a neuropsychiatric condition, a brain state or a lack thereof (e.g., coping, motivation, stress, depression or anxiety), encephalitis, or a brain dysfunction (e.g., cognitive decline or brain fog). In some embodiments, the individual is susceptible to or suffering from the mental, behavioral, or neuropsychiatric condition, and/or the symptoms thereof. In some embodiments, the individual is suffering from or susceptible to stress. In some embodiments, the individual is suffering from or susceptible to depression, anxiety, cognitive decline, or the like.

In some embodiments, the disorders or conditions are neurological disorders or conditions. In some embodiments, the disorders or conditions are neurocognitive disorders or conditions. In some embodiments, the disorders or conditions are neurodegenerative disorders or conditions. In some embodiments, the symptoms of the neurological condition are physical, behavioral, emotional, mental, or a combination thereof.

In some embodiments, the mental, the behavioral, or the neuropsychiatric condition is an attention condition or a cognitive (e.g., neurocognitive) condition. In some embodiments, the mental, the behavioral, or the neuropsychiatric condition (e.g., a Diagnostic and Statistical Manual of Mental Disorders (DSM-5) category or non-DSM-5 category disease or disorder) is selected from the group consisting of addiction, anxiety (e.g., post-traumatic stress disorder (PTSD), constructive impulsivity, a phobia, or fear), apathy, and depression (e.g., major depressive disorder).

In some preferred embodiments, the attention condition is attention deficit hyperactivity disorder (ADHD) or attention deficit disorder (ADD).

In some embodiments, the symptoms of the mental, a behavioral, or a neuropsychiatric condition are physical, behavioral, emotional, mental, or a combination thereof.

Provided in some instances herein is a method for increasing motivation in an individual. In some embodiments, the individual is suffering from or susceptible to low motivation (e.g., as a symptom of a neurocognitive or neurodevelopmental disorder), apathy, fear, phobia, constructive impulsivity, attention (e.g., or the lack thereof), cognitive conditions, or depression, comprising administering one or more 5-HT receptor agonists, or pharmaceutically acceptable salts, solvates, metabolites, derivatives, or prodrugs thereof.

In certain preferred embodiments, the cognitive condition is mild cognitive impairment, dementia, or Alzheimer's disease.

Provided herein are methods for managing or treating disorders, conditions or symptoms including but not limited to addiction (disorders), such as but not limited to alcohol abuse, substance abuse, smoking, or eating disorders. Provided herein are methods for managing or treating disorders, conditions or symptoms including but not limited to eating disorders and auditory disorders. Provided herein are methods for managing or treating disorders, conditions or symptoms including but not limited to pain, such as but not limited to chronic pain. Provided herein are methods for managing or treating disorders, conditions or symptoms including but not limited to depression, bipolar disorder, post-traumatic stress disorder (PTSD), panic disorder, phobia, schizophrenia, psychopathy, or antisocial personality disorder. Provided herein are methods for managing or treating disorders, conditions or symptoms including but not limited to impulse disorders, such as but not limited to attention deficit hyperactivity disorder (ADHD), Tourette's syndrome or autism. Provided herein are methods for managing or treating disorders, conditions or symptoms including but not limited to compulsive disorder, such as but not limited to obsessive compulsive disorder (OCD), gambling, or aberrant sexual behavior. Provided herein are methods for managing or treating disorders, conditions or symptoms including but not limited to personality disorders, such as but not limited to conduct disorder, antisocial personality, or aggressive behavior.

Further examples of the disorders, conditions and symptoms which may be managed or treated include, by way of non-limiting example:

Neurodevelopmental Disorders, such as but not limited to attention-deficit/hyperactivity disorder (ADHD), autism spectrum disorder, learning disorders and the like.

Schizophrenia Spectrum and other Psychotic Disorders, including but not limited to detachment from reality, delusions, hallucinations, and disorganized thinking and speech.

Bipolar and Related Disorders which may involve episodes of mania (periods of excessive excitement, activity, and energy) alternating with periods of depression.

Depressive Disorders which may involve feelings of extreme sadness, reduced interest in previously enjoyable activities, including but not limited to depression, severe depression, major depressive disorder (MDD), premenstrual dysphoric disorder (PMDD) and the like.

Anxiety Disorders which may involve worrying excessively about potential bad things or situations. Examples include generalized anxiety disorder (GAD), panic disorder and phobias (irrational fears of specific things) and the like.

Obsessive-Compulsive and Related Disorders which may involve repeated, unwanted urges, thoughts, or images (obsessions) and feeling driven to taking repeated actions in response to them (compulsions). Non-limiting examples include obsessive-compulsive disorder (OCD), hoarding disorder, extreme nail biting, and hair-pulling disorder (trichotillomania).

Trauma and Stressor-Related Disorders which may develop during or after stressful or traumatic life events. Non-limiting examples include posttraumatic stress disorder (PTSD) and acute stress disorder.

Dissociative Disorders wherein the sense of self is may be disrupted, such as but not limited to dissociative identity disorder, dissociative amnesia and the like.

Somatic Symptom and Related Disorders which may involve distressing and incapacitating physical symptoms with no clear medical cause. Non-limiting examples include illness anxiety disorder, somatic symptom disorder (hypochondriasis), factitious disorder and the like.

Feeding and Eating Disorders which may involve disturbances related to eating, such as but not limited to anorexia nervosa, bulimia nervosa, and binge eating disorder.

Elimination Disorders which may involve inappropriate elimination (release) of urine or stool by accident or deliberately, such as but not limited to bedwetting (enuresis).

Sleep-Wake Disorders which may involve severe sleep disorders, including but not limited to insomnia disorder, nightmare disorder, sleep apnea, and restless legs syndrome.

Disruptive, Impulse-Control, and Conduct Disorders which may involve difficulty with emotional and/or behavioral self-control, such as but not limited to kleptomania (repeated stealing), pyromania, and intermittent explosive disorder.

Substance Related addiction Disorders which may involve problems associated with excessive use of substances such as alcohol (alcohol dependence, alcoholism), tobacco products, drugs, opioids (for example, cocaine, oxycodone, morphine and the like), recreational drugs, hallucinogens and the like.

Addictive Disorders which may involve problems associated with excessive use of particular behaviors or fixations, such as but not limited to gambling disorder.

Neurocognitive Disorders which may affect the ability to think and reason, such as but not limited to traumatic brain injury (TBI), Alzheimer's disease and the like.

Personality Disorders which may involve enduring patterns of emotional instability and unhealthy behaviors that disrupt daily living and relationships. Examples include but are not limited to borderline, antisocial, and narcissistic personality disorders.

Gender Dysphoria which may involve distress caused by a person's desire to be a different gender.

Sexual Dysfunctions such as but not limited to premature ejaculation, erectile disorder, and female orgasmic disorder.

Paraphilic Disorders (sexual perversion, sexual deviation) which may involve sexual interest in atypical objects, situations, fantasies, behaviors, or individuals. Examples include but are not limited to sexual sadism disorder, voyeuristic disorder, and pedophilic disorder.

Further examples of the disorders, conditions and symptoms which may be managed or treated include by way of non-limiting examples Fragile X syndrome, Downs's syndrome, migraine headache, cluster headache, psychiatric disorders, neurodevelopmental disorders, attention-deficit/hyperactivity disorder (ADHD), autism spectrum disorder, learning disorders, schizophrenia spectrum, psychotic disorders, bipolar disorders, depression, severe depression, major depressive disorder (MDD), premenstrual dysphoric disorder (PMDD), suicidality, mood related disorders, panic disorder, panic attack, phobias, agoraphobia, selective mutism, obsessive-compulsive disorder (OCD), hoarding disorder, hair-pulling disorder (trichotillomania), excoriation (skin-picking) disorder, substance-/medication-induced obsessive-compulsive disorder, trauma related disorders, traumatic brain injury (TBI), posttraumatic stress disorder (PTSD), acute stress disorder, dissociative disorders, dissociative identity disorder, dissociative amnesia, anxiety, anxiety disorders, generalized anxiety disorder (GAD), social anxiety disorder, separation anxiety disorder, illness anxiety disorders, somatic disorders and diseases, somatic symptom disorder (hypochondriasis), factitious disorder, feeding disorders, eating disorders, anorexia, anorexia nervosa, bulimia nervosa, binge eating disorder, elimination disorders, enuresis, sleep disorders, insomnia, nightmare disorder, sleep apnea, central sleep apnea, narcolepsy, obstructive sleep apnea, hypopnea, and sleep-related hypoventilation, restless legs syndrome, jet lag, sexual dysfunction, premature ejaculation, erectile disorder, female orgasmic disorder, gender identity disorder, gender dysphoria, disruptive disorders, impulse-control disorders, conduct disorders, disruptive conduct disorders, impulse-control disorders, oppositional defiant disorder (ODD), aggression, kleptomania, pyromania, addictive disorders, substance dependence, substance abuse, alcoholism, drug addiction, opioid addiction, cocaine addiction, gambling addiction, tobacco dependence, food addiction, other forms of addiction to substances and behaviors, obesity, cognitive disorders, memory related disorders, learning related disorders, neurocognitive disorders, Alzheimer's disease, personality disorders, narcissistic personality disorders, Asperger syndrome, Tourette syndrome, Huntington's disease, Parkinson's disease, Lewy body disease, amyotrophic lateral sclerosis (ALS), Friedreich's ataxia, muscular atrophy, prion disease, dementia, vascular dementia, dementia/neurocognitive issues due to infection, dementia due to substance abuse or exposure to toxins, frontotemporal degeneration, mood disorders, delirium, aphasia, apraxia, agnosia, concussion, amnesia, anterograde amnesia, retrograde amnesia, body dysmorphic disorder, reactive attachment disorder, Fragile X syndrome, Down syndrome, migraines, migraine headache, cluster headache, cardiovascular disease, inflammatory conditions, fibromyalgia and pain.

In some embodiments, any pharmaceutical composition or formulation or 5-HT receptor agonist agent disclosed herein is administered for therapeutic application. In some embodiments, the pharmaceutical composition or formation or 5-HT receptor agonist agent is administered once per day, twice per day, three times per day or more. In certain instances, the pharmaceutical composition or formulation or 5-HT receptor agonist agent is administered daily, every day, every alternate day, five days a week, once a week, every other week, two weeks per month, three weeks per month, once a month, twice a month, three times per month, or more. In some embodiments, the pharmaceutical composition or formulation or 5-HT receptor agonist agent is administered for at least 1 month, 2 months, 3 months, 4 months, 5 months, 6 months, 7 months, 8 months, 9 months, 10 months, 11 months, 12 months, 18 months, 2 years, 3 years, or more.

In some embodiments, one or more pharmaceutical compositions are administered simultaneously, sequentially, or at an interval period of time. In some embodiments, one or more pharmaceutical compositions are administered simultaneously. In some cases, one or more pharmaceutical compositions are administered sequentially. In additional cases, one or more pharmaceutical compositions are administered at an interval period of time (e.g., the first administration of a first pharmaceutical composition is on day one followed by an interval of at least 1, 2, 3, 4, 5, or more days prior to the administration of at least a second pharmaceutical composition).

In some embodiments, two or more different pharmaceutical compositions are co-administered. In some instances, the two or more different pharmaceutical compositions are co-administered simultaneously. In some cases, the two or more different pharmaceutical compositions are co-administered sequentially without a gap of time between administrations. In other cases, the two or more different pharmaceutical compositions are co-administered sequentially with a gap of about 0.5 hour, 1 hour, 2 hour, 3 hour, 12 hours, 1 day, 2 days, or more between administrations.

In some embodiments, the amount of a given agent that corresponds to such an amount varies depending upon factors such as the particular compound, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day.

in some embodiments, described herein are compositions and formulations comprising a 5-HT receptor agonist active ingredient. In some embodiments, the formulation comprising 5-HT receptor agonist, or pharmaceutically acceptable salt, solvate, metabolite, derivative, or prodrug thereof, has: an enhanced bioavailability and efficacy, a lower administration dose, a lower cytotoxicity, decreased side effects, or the like.

In some embodiments, the formulation comprises one or more 5-HT receptor agonist, or a pharmaceutically acceptable salt, solvate, metabolite, derivative, or prodrug thereof. In some embodiments, the one or more 5-HT receptor agonist, or a pharmaceutically acceptable salt, solvate, metabolite, derivative, or prodrug thereof is a 5HT_{2A} receptor agonist.

In some embodiments, the one or more 5-HT receptor agonist, or the pharmaceutically acceptable salt, solvate, metabolite, derivative, or prodrug thereof, is a hallucinogenic compound (e.g., wherein the hallucinogenic compound produces a hallucinogenic effect (e.g., an adverse event, a clinically important effect (e.g., clinically important impairment of the individual, altered (e.g., visual, auditory, body, time and space) perception, altered cognition, impaired attention, drowsiness, and/or confusion)) in the individual in need thereof at or above the hallucinogenic threshold (e.g., at or above a Cₘₐₓ above the hallucinogenic effective threshold)).

In some embodiments, the one or more 5-HT receptor agonist is psilocybin or psilocin, or a pharmaceutically acceptable salt, solvate, metabolite, derivative, or prodrug thereof. In some embodiments, the one or more 5-HT receptor agonist is psilocin.

In some embodiments, provided herein is a formulation configured to maintain a level of an active 5-HT receptor agonist at or above a minimum therapeutically effective threshold of the active 5-HT receptor agonist in an individual (e.g., in a biological sample (e.g., serum, plasma, or whole blood) of the individual) for more than or equal to two hours (e.g., more than 2 hours, more than 12 hours, more than 1 day, more than 7 days, or more than 14 days). In some embodiments, provided herein is a formulation configured to maintain a level of an active 5-HT receptor agonist below a hallucinogenic threshold (e.g., below a threshold that produces an adverse event (e.g., a clinically important effect (e.g., clinically important impairment of the individual), altered (e.g., visual, auditory, body, time and space) perception, altered cognition, impaired attention, drowsiness, confusion, or the like) of the active 5-HT receptor agonist in an individual (e.g., in a biological sample (e.g., serum, plasma, or whole blood) of the individual) for more than or equal to two hours (e.g., more than 2 hours, more than 12 hours, more than 1 day, more than 7 days, or more than 14 days). In some embodiments, provided herein is a formulation configured to maintain a level of an active 5-HT receptor agonist (i) at or above a minimum therapeutically effective threshold and (ii) below a hallucinogenic threshold (e.g., below a threshold that produces an adverse event (e.g., a clinically important effect (e.g., clinically important impairment of the individual), altered (e.g., visual, auditory, body, time and space) perception, altered cognition, impaired attention, drowsiness, confusion, or the like) of the active 5-HT receptor agonist in the individual (e.g., in a biological sample (e.g., serum, plasma, or whole blood) of the individual) for more than or equal to two hours (e.g., more than 2 hours, more than 12 hours, more than 1 day, more than 7 days, or more than 14 days).

In some embodiments, provided herein is a formulation configured to release the active 5-HT receptor agonist at a dose sufficient to provide a Cₘₐₓ below the hallucinogenic effective threshold of the active 5-HT receptor agonist and a Cₘᵢₙ of at least the therapeutically effective threshold of the active 5-HT receptor agonist in the individual.

In some embodiments, provided herein is a formulation configured to provide a maximum plasma concentration (Cₘₐₓ) of the active 5-HT receptor agonist of about 0.1 ng/mL to about 20 ng/mL (e.g., about 0.5 ng/mL to about 20 ng/mL, about 1 ng/mL to about 15 ng/mL, about 2 ng/mL to about 12 ng/mL, or the like) in the individual.

In some embodiments, provided herein is a formulation configured to provide a plasma concentration of the active 5-HT receptor agonist of at least 0.1 ng/mL (e.g., at least 0.2 ng/mL, at least 0.3 ng/mL, at least 0.5 ng/mL, or the like) in the individual for at least 3 hours (e.g., at least 12 hours, at least 24 hours, at least 36 hours, at least 48 hours, at least 72 hours, at least 96 hours, at least 120 hours, at least 144 hours, or the like).

In some embodiments, the composition or formulation is an oral formulation. In some instances, the oral formulation comprising 5-HT receptor agonist, or pharmaceutically acceptable salt, solvate, metabolite, derivative, or prodrug thereof, has: an enhanced bioavailability and efficacy, a lower administration dose, a lower cytotoxicity, and decreased side effects.

In some embodiments, the pharmaceutically acceptable excipient is selected from the group consisting of fillers, binders, suspending agents, disintegrants, lubricants, and combinations thereof.

In some embodiments, 5HT receptor agonists or pharmaceutical compositions or formulations described herein are administered to a subject by multiple administration routes, including but not limited to, oral, parenteral (e.g., intravenous, subcutaneous, intramuscular), intranasal, inhalation, buccal, topical, rectal, or transdermal administration routes.

=In some embodiments, the one or more 5-HT receptor agonist, or a pharmaceutically acceptable salt, solvate, metabolite, derivative, or prodrug thereof, is administered to the individual in need thereof as an oral formulation, an intravenous formulation, a dermal formulation, a buccal formulation, a nasal formulation, or an inhalation formulation.

In some embodiments, pharmaceutical compositions described herein, which include 5HT receptor agonist, or pharmaceutically acceptable salt, solvate, metabolite, derivative, or prodrug thereof, are formulated into any suitable dosage form.

In some embodiments, the pharmaceutical composition for oral use is a tablet, a pill, a powder, a capsule, solid dispersion, solid solution, bioerodible dosage form, pellets, granules, or an aerosol, or the like.

For oral administration, the pharmaceutical compositions disclosed herein are, in some instances, formulated readily by combining the active compound(s) with pharmaceutically acceptable carriers. In some instances, such formulations comprise pharmaceutically acceptable carriers including solid diluents or fillers, sterile aqueous media and various non-toxic organic solvents. In some instances, the compositions disclosed herein are present at concentration levels of at least about 0.5% by weight of the total composition of oral dosage forms, in an amount sufficient to provide a desired unit of dosage.

In some embodiments, the amount of 5HT receptor agonist, or pharmaceutically acceptable salt, solvate, metabolite, derivative, or prodrug thereof in a pharmaceutical composition provided herein is at least 1 mg/mL (e.g., 1 mg/mL or more, 5 mg/mL or more, 10 mg/mL or more, 15 mg/mL or more, 20 mg/mL or more, 25 mg/mL or more, or 30 mg/mL or more). In some embodiments, the amount of 5HT receptor agonist, or pharmaceutically acceptable salt, solvate, metabolite, derivative, or prodrug thereof in a pharmaceutical composition provided herein is at most 30 mg/ml (e.g., 30 mg/mL or less, 25 mg/mL or less, 20 mg/mL or less, 15 mg/mL or less, 10 mg/mL or less, 5 mg/mL or less, or 1 mg/mL or less). In some embodiments, the amount of 5HT receptor agonist, or pharmaceutically acceptable salt, solvate, metabolite, derivative, or prodrug thereof in a pharmaceutical composition provided herein is 1 mg/mL to 30 mg/mL. In some embodiments, the amount of 5HT receptor agonist, or pharmaceutically acceptable salt, solvate, metabolite, derivative, or prodrug thereof in a pharmaceutical composition provided herein is about 0.1 mg/ml to about 10 mg/ml.

In some embodiments, the amount of 5HT receptor agonist, or pharmaceutically acceptable salt, solvate, metabolite, derivative, or prodrug thereof in a pharmaceutical composition provided herein is at least about 0.001 mg (e.g., 0.001 mg or more, 0.01 mg or more, 0.1 mg or more, 1 mg or more, 10 mg or more, or 100 mg or more) In some embodiments, the amount of 5HT receptor agonist, or pharmaceutically acceptable salt, solvate, metabolite, derivative, or prodrug thereof in a pharmaceutical composition provided herein is at most about 100 mg (e.g., 100 mg or less, 10 mg or less, 1 mg or less, 0.1 mg or less, 0.01 mg or less, or 0.001 mg or less). In some embodiments, the amount of 5HT receptor agonist, or pharmaceutically acceptable salt, solvate, metabolite, derivative, or prodrug thereof in a pharmaceutical composition provided herein is about 0.001 mg to about 100 mg. In some embodiments, the amount of 5HT receptor agonist, or pharmaceutically acceptable salt, solvate, metabolite, derivative, or prodrug thereof in a pharmaceutical composition provided herein is about 0.005 mg to about 15 mg. In some embodiments, the amount of 5HT receptor agonist, or pharmaceutically acceptable salt, solvate, metabolite, derivative, or prodrug thereof in a pharmaceutical composition provided herein is about 0.01 mg to about 5 mg. In some embodiments, the amount of 5HT receptor agonist, or pharmaceutically acceptable salt, solvate, metabolite, derivative, or prodrug thereof in a pharmaceutical composition provided herein is about 0.05 mg to about 2.5 mg.

In some embodiments, the amount of 5HT receptor agonist, or pharmaceutically acceptable salt, solvate, metabolite, derivative, or prodrug thereof in a pharmaceutical composition provided herein is at least about 0.001 mg/kg (e.g., 0.001 mg/kg or more, 0.01 mg/kg or more, 0.1 mg/kg or more, 1 mg/kg or more, 10 mg/kg or more, or 100 mg/kg or more) In some embodiments, the amount of 5HT receptor agonist, or pharmaceutically acceptable salt, solvate, metabolite, derivative, or prodrug thereof in a pharmaceutical composition provided herein is at most about 100 mg/kg (e.g., 100 mg/kg or less, 10 mg/kg or less, 1 mg/kg or less, 0.1 mg/kg or less, 0.01 mg/kg or less, or 0.001 mg/kg or less). In some embodiments, the amount of 5HT receptor agonist, or pharmaceutically acceptable salt, solvate, metabolite, derivative, or prodrug thereof in a pharmaceutical composition provided herein is about 0.001 mg/kg to about 100 mg/kg. In some embodiments, the amount of 5HT receptor agonist, or pharmaceutically acceptable salt, solvate, metabolite, derivative, or prodrug thereof in a pharmaceutical composition provided herein is about 0.005 mg/kg to about 10 mg/kg. In some embodiments, the amount of 5HT receptor agonist, or pharmaceutically acceptable salt, solvate, metabolite, derivative, or prodrug thereof in a pharmaceutical composition provided herein is about 0.01 mg/kg to about 5 mg/kg. In some embodiments, the amount of 5HT receptor agonist, or pharmaceutically acceptable salt, solvate, metabolite, derivative, or prodrug thereof in a pharmaceutical composition provided herein is about 0.05 mg/kg to about 1 mg/kg.

In some embodiments, the amount of 5-HT receptor agonist, or pharmaceutically acceptable salt, solvate, metabolite, derivative, or prodrugs thereof in a pharmaceutical composition provided herein is at least about 1% w/w (e.g., 1% or more, 10% or more, 20% or more, 30% or more, 40% or more, or 50% or more) (e.g., of the solids in an oral formulation). In some embodiments, the amount of 5-HT receptor agonist, or pharmaceutically acceptable salt, solvate, metabolite, derivative, or prodrugs thereof in a pharmaceutical composition provided herein is at most about 50% w/w (e.g., 50% w/w or less, 40% w/w or less, 30% w/w or less, 20% w/w or less, 10% w/w or less, or 1% w/w or less) (e.g., of the solids in an oral formulation).

In some embodiments, a brain evaluation (e.g., a brain test) provided herein is performed using a wearable brain imaging device (e.g., on the individual).

In some embodiments, one or more brain activity or the lack thereof is measured with the wearable brain imaging device and recorded to a local memory or over a network using a mobile device (e.g., through a mobile application).

In some embodiments, one or more cardiovascular activity (e.g., heart rate) is measured with an electrode sensitive voltage or sensor (e.g., a photodiode) sensing an optical measure from visible light or infrared light. In some embodiments, the electrode sensitive voltage or sensor is a separate electrode sensitive voltage or sensor (e.g., from the brain activity measuring device).

Provided in some embodiments herein is a method for monitoring the effectiveness of a 5-HT receptor agonist provided herein (e.g., psilocybin), comprising measuring (e.g., on a network (e.g., WiFi, cloud, BLE, 3/4/5G)) one or more brain response using a node or a wearable brain imaging device (e.g., an EEG). In some embodiments, the patient listens to paired or sustained auditory stimuli (e.g., at home, at work, or at school). In some embodiments, the auditory stimuli being delivered from an application on a (e.g., mobile) device (e.g., cellular phone, tablet, or the like).

Provided in some embodiments herein is a method for using Bayesian statistics and methods to estimate and predict confidence in the effectiveness of a treatment or dose of a psychedelic compound provided herein based on historical brain imaging data in a database for particular patient types.

Provided in some embodiments herein is a method of imaging brain activity (e.g., voltage) in an individual using a wearable device (e.g., EEG (e.g., multi-electrode EEG device)), the wearable device having two or more electrodes that make contact with the forehead of the individual for at least thirty seconds and up to twenty minutes.

Provided in some embodiments herein is a method for optically imaging brain activity in an individual (e.g., from a wearable device) to determine, track, or optimize the effectiveness of a psychedelic compound, wherein a brain signal is identified by transmission of visible light (400-680 nanometers) or infrared light (greater than or equal to 680 nanometers) across the skin into the brain to collect reflected photons by a sensor (e.g., a photodiode or CMOS).

Provided in some embodiments herein is a method of acoustically imaging brain activity in an individual (e.g., from a wearable device) to determine, track, or optimize the effectiveness of a psychedelic compound, wherein a brain signal is identified by transmission of ultrasound (e.g., having an acoustic frequency of greater than or equal to one megahertz) (e.g., across the skin and skull) into the brain to collect reflected ultrasound waves by a sensor (e.g., a piezoelectric resonant material (e.g., a PZT, a CMUT, a PMUT).

In some embodiments, the method further comprises using artificial intelligence (e.g., deep convolution neural networks) algorithms and/or machine learning processes to classify, cluster, or recognize patterns or relationships amongst sensory-evoked (e.g., auditory or visual stimuli) brain activity or resting state brain activity acquired by brain imaging methods and patient reported outcomes (e.g., changes in mood, anxiety, motivation, or memory) for optimizing the dosing schedule or treatment paradigm or a patient undergoing treatment with psychedelic substances (e.g. psilocybin).

Provided in some embodiments herein is a computer-implemented method for identifying a therapeutically effective dose of a 5-HT receptor agonist administered to an individual, the method comprising:
a) administering the 5-HT receptor agonist to the individual;
b) emitting one or more (e.g., auditory or visual) to the individual;
c) receiving, from a brain imaging device (e.g., an EEG), a brain response; and
d) identifying the therapeutically effective dose of the 5-HT receptor agonist based at least in part on the one or more (e.g., auditory or visual) stimulus and the brain response.

In some embodiments the method further comprises receiving, from the individual, an emotional data, and wherein the therapeutically effective dose of the 5-HT receptor agonist is further determined based on the emotional data. In some embodiments, the emotional data comprises a mood rating, a sleep rating, a stress rating, an anxiety rating, a memory rating, or any combination thereof.

In some embodiments, two or more of (a), (b), and (c) are performed simultaneously.

In some embodiments, two or more of (a), (b), and (c) are performed sequentially.

In some embodiments, the method further comprises transmitting the therapeutically effective dose of the 5-HT receptor agonist to the individual, a caregiver, or both.

In some embodiments, the therapeutically effective dose of the 5-HT receptor agonist is transmitted by a mobile device (e.g., cellular phone, tablet). In some embodiments, the therapeutically effective dose of the 5-HT receptor agonist is transmitted over a wireless network (e.g., WiFi, cloud, BLE, 3/4/5G). in some embodiments, the therapeutically effective dose of the 5-HT receptor agonist is determined by a machine learning algorithm.

Provided in some embodiments herein is a computer-implemented system for identifying a therapeutically effective dose of a 5-HT receptor agonist administered to an individual, the system comprising: a digital processing device comprising: at least one processor, an operating system configured to perform executable instructions, a memory, and a computer program including instructions executable by the digital processing device to create an application configured to perform at least the following:
a) receiving an indicator of the 5-HT receptor agonist administered to the 5-HT receptor agonist provided to the individual;
b) directing a device (e.g., an auditory or visual device) to emit one or more (auditory or visual) stimulus to the individual;
c) receiving, from a brain imaging device (e.g., EEG), a brain response; and
d) identifying the therapeutically effective dose of the 5-HT receptor agonist based at least in part on the one or more (auditory or visual) stimulus and the brain response.

In some embodiments, the application is further configured to perform receiving, from the individual, an emotional data, and wherein the therapeutically effective dose of the 5-HT receptor agonist is further determined based on the emotional data. In some embodiments, the emotional data comprises a mood rating, a sleep rating, a stress rating, an anxiety rating, a memory rating, or any combination thereof. In some embodiments, the application is configured to simultaneously perform two or more of steps (a), (b), and (c). In some embodiments, the application is configured to sequentially perform two or more of steps (a), (b), and (c).

In some embodiments, the application is further configured to transmit the therapeutically effective dose of the 5-HT receptor agonist to the individual, a caregiver, or both. In some embodiments, the application directs the transmission of the therapeutically effective dose of the 5-HT receptor agonist by a mobile device (e.g., cellular phone, tablet). In some embodiments, the application directs the transmission of the therapeutically effective dose of the 5-HT receptor agonist over a wireless network (e.g., WiFi, cloud, BLE, 3/4/5G). In some embodiments, the therapeutically effective dose of the 5-HT receptor agonist is determined by a machine learning algorithm.

Provided in some embodiments herein is non-transitory computer-readable storage media encoded with a computer program including instructions executable by a processor to create an application for identifying a therapeutically effective dose of a 5-HT receptor agonist administered to an individual, the application configured to perform at least the following:
a) receiving an indicator of the 5-HT receptor agonist administered to the individual;
b) directing a device (e.g., an auditory device or a visual device) to emit one or more (e.g., auditory or visual) stimulus to the individual;
c) receiving, from a brain imaging device (e.g., EEG), a brain response; and
d) identifying the therapeutically effective dose of the 5-HT receptor agonist based at least in part on the auditory stimulus and the brain response.

In some embodiments, the application is further configured to perform receiving, from the individual, an emotional data, and wherein the therapeutically effective dose of the 5-HT receptor agonist is further determined based on the emotional data. In some embodiments, the emotional data comprises a mood rating, a sleep rating, a stress rating, an anxiety rating, a memory rating, or any combination thereof.

In some embodiments, the application is configured to simultaneously perform two or more of steps (a), (b), and (c). In some embodiments, the application is configured to sequentially perform two or more of steps (a), (b), and (c).

In some embodiments, the application is further configured to transmit the therapeutically effective dose of the 5-HT receptor agonist to the individual, a caregiver, or both. In some embodiments, the application directs the transmission of the therapeutically effective dose of the 5-HT receptor agonist by a mobile device (e.g., cellular phone, tablet). In some embodiments, the application directs the transmission of the therapeutically effective dose of the 5-HT receptor agonist over a wireless network (e.g., WiFi, cloud, BLE, 3/4/5G). In some embodiments, the therapeutically effective dose of the 5-HT receptor agonist is determined by a machine learning algorithm.

Provided in some embodiments herein is a computer-implemented system for identifying a therapeutically effective dose of a 5-HT receptor agonist administered to an individual, the system comprising: a digital processing device comprising: at least one processor, an operating system configured to perform executable instructions, a memory, and a computer program including instructions executable by the digital processing device to create an application configured to perform at least the following:
a) applying a first machine learning algorithm to a plurality of 5-HT receptor agonist doses (e.g., dose responses), a plurality of (e.g., auditory or visual) stimuli, and brain responses to identifying the therapeutically effective dose of the 5-HT receptor agonist;
b) receiving verified emotional data regarding an emotional effectiveness of the 5-HT receptor agonist; and
c) feeding back the verified data to improve the first machine learning algorithm's calculation over time.

In some embodiments, the first machine learning algorithm is trained by a neural network comprising:
a) a first training module for creating a first training set comprising a set of 5-HT receptor agonist doses (e.g., dose responses), each dose associated with one (e.g., auditory or visual) stimulus, and one brain response;
b) a first training module training the neural network using the first training set;
c) a second training module creating a second training set for second stage training comprising the first training set and the psychedelic treatments incorrectly detected as having a positive effectiveness on the individual after the first stage of training; and
d) training the neural network using the second training set.

Provided in some embodiments herein is a non-transitory computer-readable storage media encoded with a computer program including instructions executable by a processor to create an application for identifying a therapeutically effective dose of a 5-HT receptor agonist administered to an individual, the application configured to perform at least the following:
a) applying a first machine learning algorithm to a plurality of 5-HT receptor agonist doses (e.g., dose responses), a plurality of (e.g., auditory or visual) stimuli, and brain responses to identify the therapeutically effective dose of the 5-HT receptor agonist;
b) receiving verified emotional data regarding an emotional effectiveness of the 5-HT receptor agonist; and
c) feeding back the verified data to improve the first machine learning algorithm's calculation over time.

In some embodiments, the first machine learning algorithm is trained by a neural network comprising:
a) a first training module creating a first training set comprising a set of 5-HT receptor agonist doses (e.g., dose responses), each dose associated with one (e.g., auditory or visual) stimulus, and one brain response;
b) a first training module training the neural network using the first training set; c) a second training module creating a second training set for second stage training comprising the first training set and the 5-HT receptor agonist doses (e.g., dose responses) incorrectly detected as having a positive effectiveness on the individual after the first stage of training; and
d) training the neural network using the second training set.

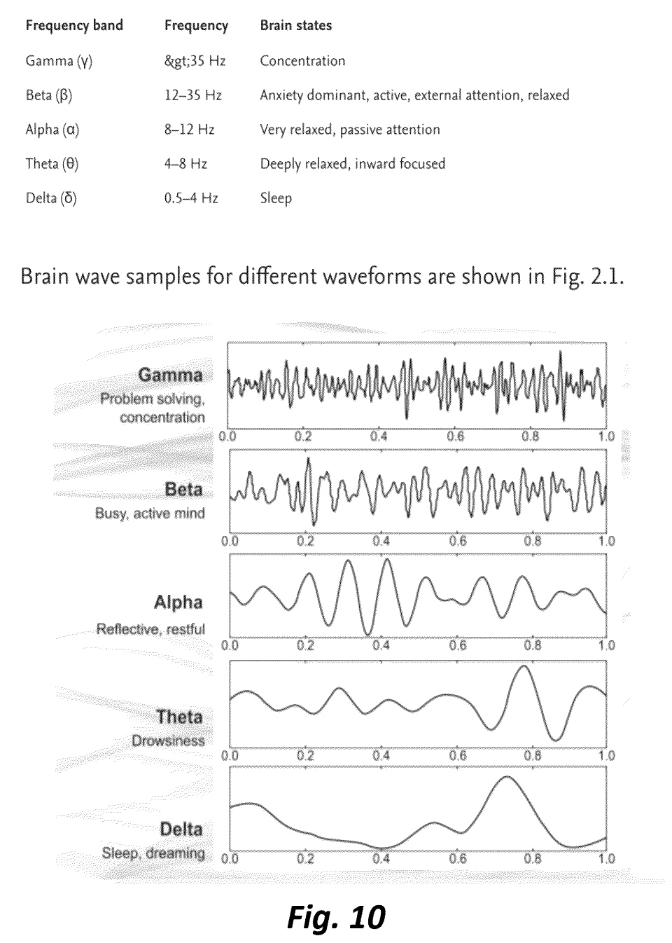

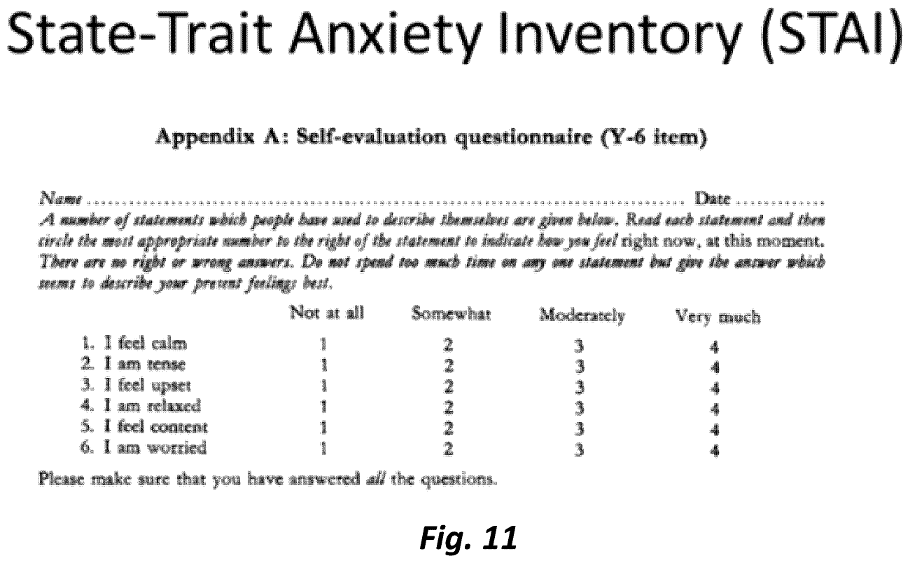

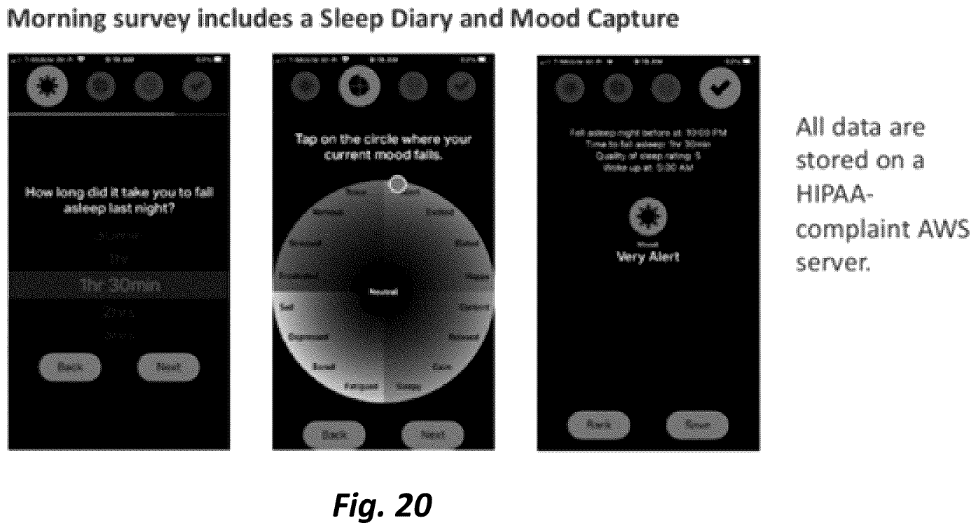

In some embodiments, the application can be used to monitor brain activity of subjects. The application can pair to a subject's device (e.g., EEG device). A subject can log onto the application using their subject ID. The application can be designed to acquire data regarding stress and anxiety using a modified version of the Short State Trait Anxiety Inventory (STAI-6) once per day. An example of a STAI-6 is shown in FIG. 11. The application can collect data on mood using a modified version of the Russel Mood Circumplex twice daily. The application can record information about sleep time and quality, fatigue, and drug use in the morning and evening using a modified version of the NIH Sleep Diary. Examples of sleep diary questions are shown in FIG. 20.

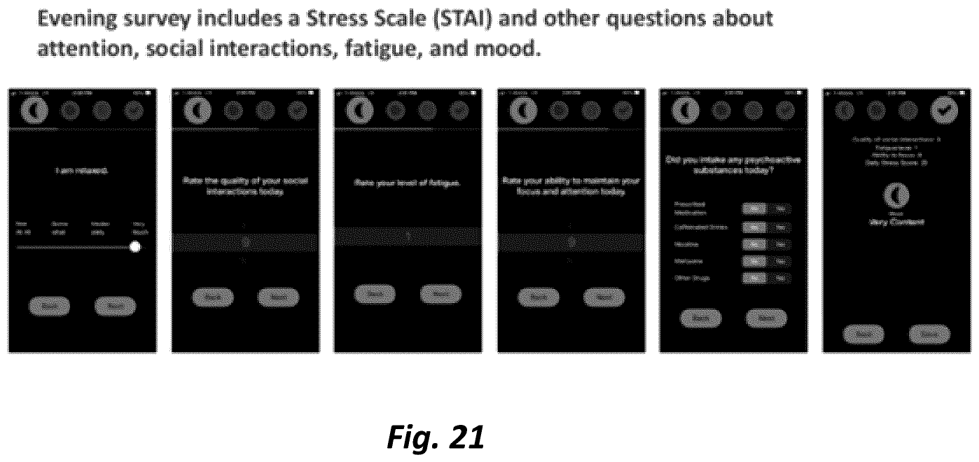

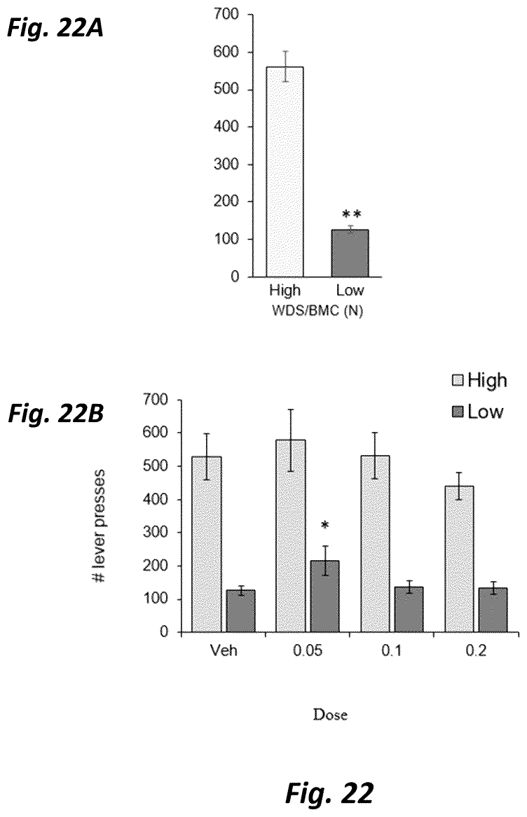

The application can work with a custom engineered, at home EEG device (e.g., a CGX, LLC device) that records activity from the prefrontal cortex using a wearable, low-risk device. The application can pair automatically to the device using Bluetooth. The rechargeable, battery-powered, wearable EEG device can use disposable, biocompatible, snap biopotential sensor electrodes. Users can record resting state at any time they wish in one-minute epochs. The option to record resting state brain activity in the morning and evening can be built into the questionnaires. Subjects may be asked to record 1-minute epochs of resting state EEG activity in the morning or evening at least 4 times per week. Subjects can record resting state EEG activity as often as desired. An example questionnaire for recording brain activity is shown in FIG. 21. Further examples of recording brain activity in the morning and evening in the application are shown in FIG. 22.

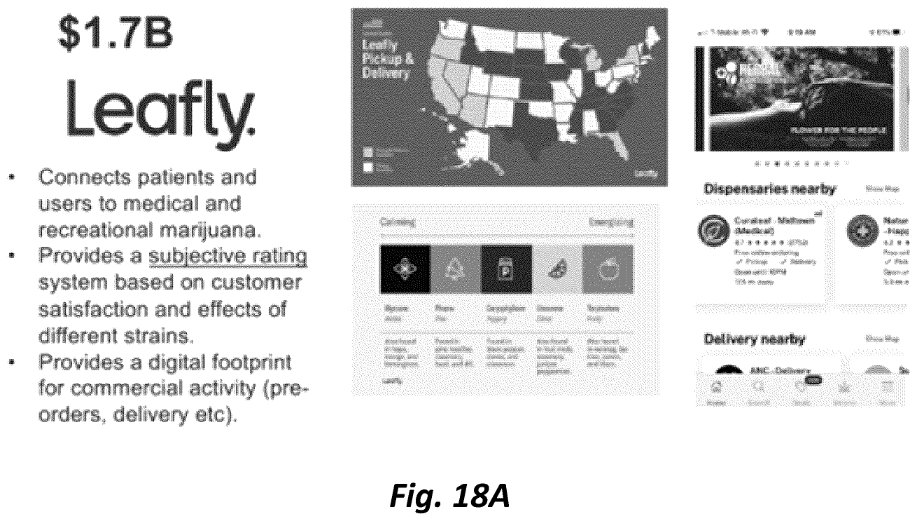

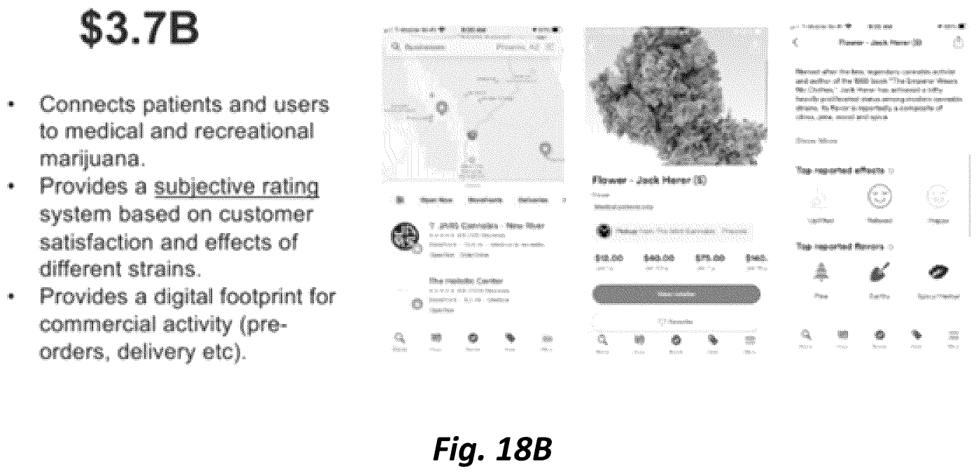

The application can connect patients and users to medical and recreational marijuana, as shown in FIGS. 18A-18B.

The application can record information about sleep time and quality, fatigue, and drug use in the morning and evening using a modified version of the NIH Sleep Diary. Examples of sleep diary questions are shown in FIG. 20.

The application can also record information about coping. For example, the application can utilize an Approach-Avoid metric to measure coping.

In some embodiments, auditory stimuli comprises auditory tasks. Auditory tasks may comprise P50 paired click auditory suppression, Mismatch Negativity (MMN), and/or Auditory Steady State Response (ASSR). The P50 task can involve the subject listening to 120 paired auditory clicks (1 msec each) occurring 500 msec apart. Each pairing can occur 10 sec apart. The estimated time to complete the passive P50 task is 20 min. The ASSR task can require subjects to listen to a series of tones using a 500 or 1000 Hz carrier frequency modulated at 20 or 40 Hz for 1 second each. Each 1 sec tone can be separated by a three second inter-stimulus interval. A total of 100 tones at 20 and 40 Hz each can be delivered. The ASSR task can be expected to last 15 minutes. The MMN task can require subjects to listen to 500 tones (1 msec) spaced 1 second apart. There can be 400 tones at a standard frequency (750 Hz) and 100 randomly spaced odd-ball tones (1500 Hz). The MMN task can be expected to take 10 minutes. Subjects can also undergo a final resting state period of EEG collection where they lay down with their eyes closed for 10 minutes. EEG recordings will also include other biosensors for recording heart rate activity during testing.

In some embodiments, visual stimuli may include one or more visual tasks comprising an Emotional Flanker Task and/or a Continuous Performance Test (CPT). Following EEG assessments following auditory tasks as described above, subjects can undergo a series of computerized cognitive attention and emotional tasks while recording EEG. Subjects can first complete an Emotional Flanker Test that uses neutral faces or an angry face as a background distractor on classic flanker stimuli assessing congruent, incongruent, and neutral conditions. A stimulus from each condition can be presented 50 times and a total of 250 stimuli can be presented over 10 minutes. Subjects can then take a Conner's Continuous Performance Test that lasts 14 minutes to assess attention and cognitive control.

The foregoing ranges are merely suggestive, as the number of variables in regard to an individual treatment regime is large, and considerable excursions from these recommended values are not uncommon. Such dosages is altered depending on a number of variables, not limited to the activity of the compound used, the disease or condition to be treated, the mode of administration, the requirements of the individual subject, the severity of the disease or condition being treated, and the judgment of the practitioner.

### EXAMPLES

These examples are provided for illustrative purposes only and not to limit the scope of the claims provided herein.

### Example 1: Mismatch Negativity (MMN) and Auditory Steady State Response (ASSR)

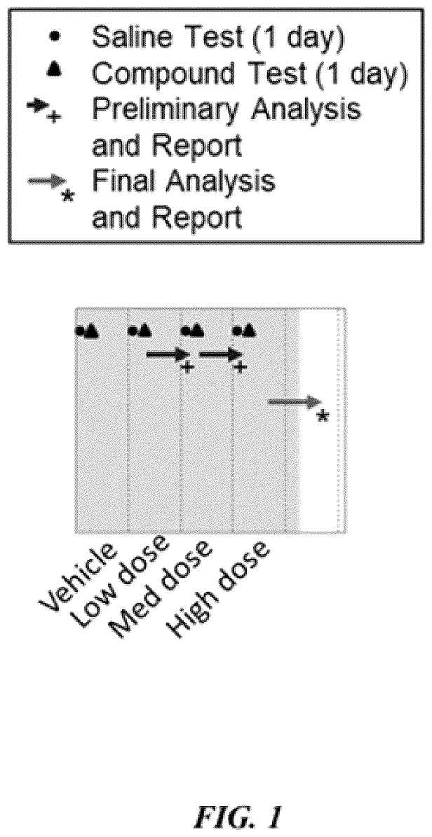

Fourteen mice with a history of testing with scopolamine and memantine, containing implanted with a frontal screw for MMN measurement (in mm: AP 1.5; ML 0.5) and an auditory screw for ASSR measurement (in mm: AP −2.8; ML 4.2), are administered an ascending dose of psilocybin. As the mice have been tested with the MMN-ASSR protocol, they are not further habituated prior to psilocybin testing. The ascending dose protocol is summarized below and in FIG. 1:
Week 1: Day 1: Saline, Day 2: Vehicle
Week 2: Day 1:Saline, Day 2: Low Dose
Week 3: Day 1: Saline, Day 2: Medium Dose
Week 4: Day 1: Saline, Day 2: High Dose

The high, medium and low doses are not intended to refer to absolute doses, but rather a dose relative to one another. For example, a larger amount of psilocybin is administered as a high dose than the amount of psilocybin is administered as medium dose, which are each a larger dose of psilocybin than the amount of psilocybin administered as a low dose.

Each animal is administered the same compound and dose each day (e.g., saline on day 1, vehicle or compound on day 2). Each animal is tested each week (or longer as necessary). Each animal is subjected to a preliminary examination each week after saline administration (day 1) to determine whether the effects (of the compound) are still present from the prior week.

In particular, on Day 1, animals are subcutaneously administered saline at t=−15 minutes prior to recording start. On day 2, animals are dosed with Vehicle or compound (depending on the week) at T=−15 minutes prior to recording start.

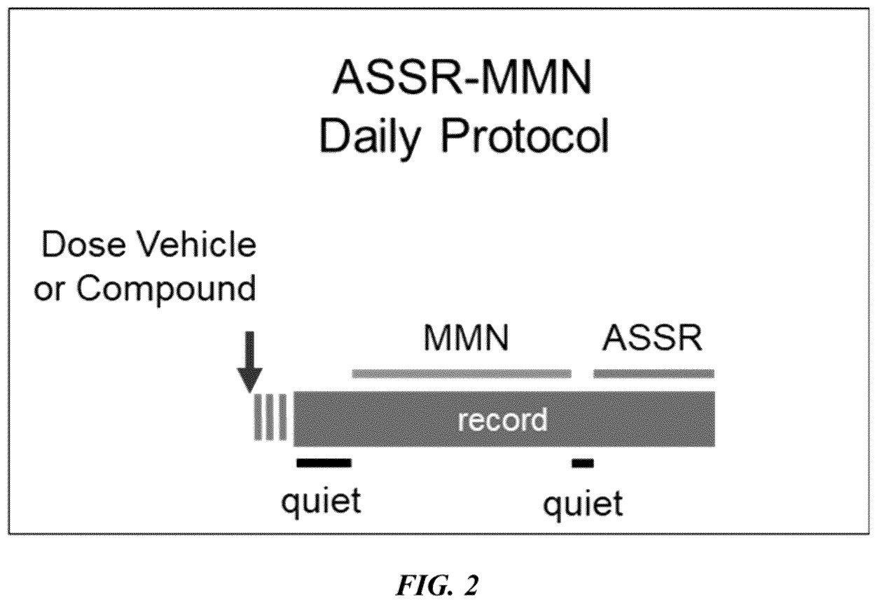

An MMN-ERP daily protocol is depicted in FIG. 2. The recording session starts at T=0 minutes, and there is a first quiescent period of 10 minutes (starting at T=0). After the first quiescent period, a MMN test (summarized below) is performed on each animal for 20 minutes. Subsequent to the MMN test, there is a second quiescent period of 2 minutes. Following the second quiescent period, a ASSR test is performed on each animal for about 11 minutes.

MMN test: the animals are subjected to auditory frequencies in a flip-flop design where both frequencies are standard and deviant (6 kilohertz (kHz) and 9 kHz tones). Each tone duration is 10 milliseconds (ms), with a 300 ms interstimulation interval (ISI). The test includes 100 trials (100 deviants, 19000 standards) where the deviant is randomly presented to each animal.

ASSR test: the animals are subjected to auditory frequencies for 500 ms in a click train design presented with an ISI of 2 seconds (s). The click train comprises 2 ms white noise clicks. Each animal is subjected to 80 trials per frequency of four different frequencies (20 hertz (Hz), 30 Hz, 40 Hz, and 80 Hz) where the typical amplitude of each frequency is 70 decibels (dB).

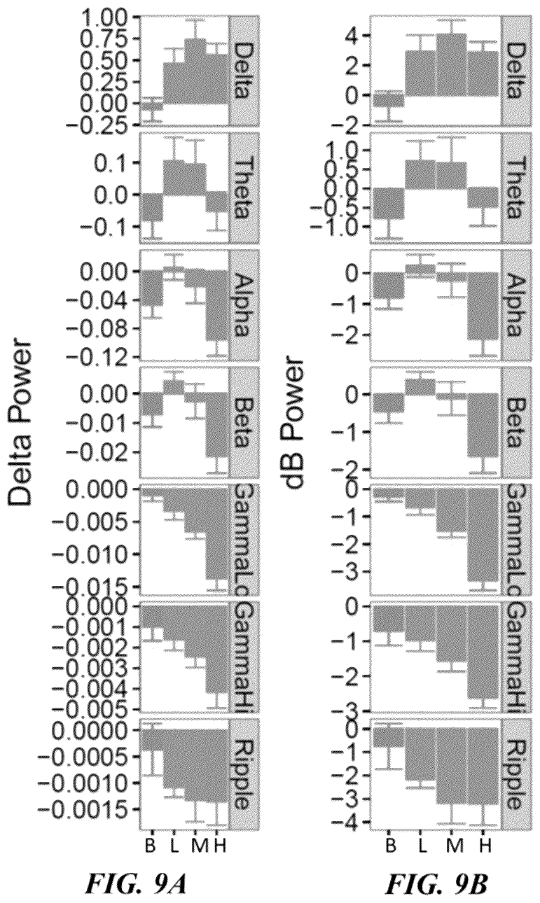

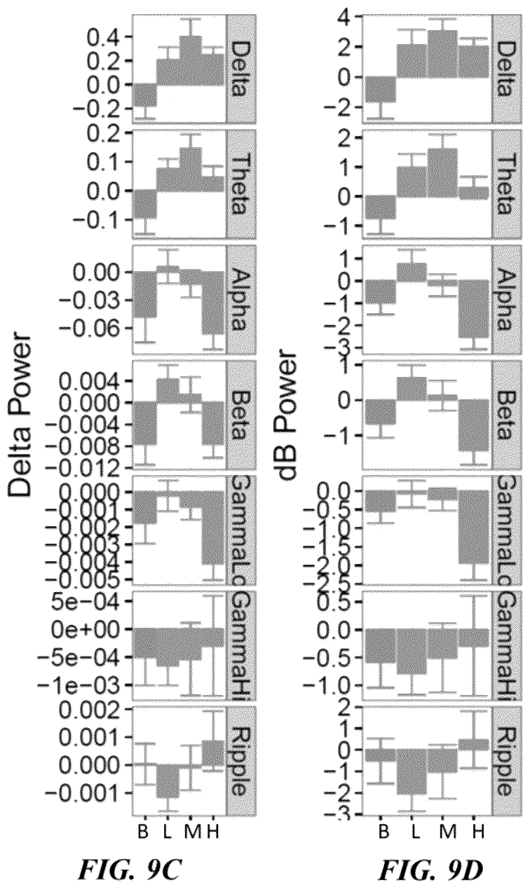

The ongoing behavior (e.g., speed, total number of grooming bouts, elongation factor, distance traveled, head bobbing, etc.) of each mouse is assessed over each daily recording session (e.g., see FIG. 3). The behavioral results (e.g., FIG. 3) correspond well with and suggest validation of the electroencephalogram (EEG) results described herein. The MMN is reported as the amplitude of the difference (DIFF) between the standard tone (STD) and deviant tone (DEV) waveforms (e.g., see FIG. 6A and FIG. 6B). The MNN is recorded for both recording regions (e.g., frontal cortex region recordings shown in FIG. 6A and FIG. 6B). The amplitude of the standard and deviant waveforms are also calculated. As shown in FIG. 6A and FIG. 6B, there is a larger difference between the standard tone waveform and the deviant form waveform with higher doses of psilocybin, suggesting a stabilization (i.e., asynchronization) in brain activity, which indicates a normalizing brain function. The ASSR is recorded as the average amplitude of both Evoked Power and Phase Locking Factor at each frequency (20 Hz, 30 Hz, 40 Hz, and 8 Hz) for control (saline) and compound (psilocybin). The difference between the control and compound recording for Evoked Power (e.g., see FIG. 4A and FIG. 4B) and Phase Locking Factor (e.g., see FIG. 5A and FIG. 5B) is determined. As shown in FIG. 4A, FIG. 4B, FIG. 5A, and FIG. 5B, the change in the difference (between the control and compound recording) across a particular frequency indicates the dose at which brain activity becomes asynchronized, which indicates the therapeutically effective dose. Ongoing spectral power (quantitative electroencephalogram (qEEG); Welch's method of power spectral density, fast Fourier transform (FFT)-based) is calculated as a function of time over each daily session before and between stimulation periods. The average power spectral density (PSD) for both time windows is visualized (e.g., see FIG. 7A, FIG. 7B, FIG. 8A, and FIG. 8B). In FIG. 7A, FIG. 7B, FIG. 8A, and FIG. 8B, the darker regions are indicative of a decrease in general brain activity, which suggests a stabilization of general brain activity. As the figures show, higher dose corresponds with a decrease in general brain activity (which are darker regions), which indicates there is a loss in spectral power, suggesting brain activity asynchronized (i.e., normalized brain function). The average power is calculated in alpha, beta, delta, gamma, theta, and ripple frequency bands and statistically analyzed for both time windows. The results are analyzed by a 1Way RM ANOVA, corrected for sphericity (where appropriate) with Bonferroni corrected post hoc tests (e.g., see FIG. 9A, FIG. 9B, FIG. 9C, and FIG. 9D).

### Example 2: Brain Activity

Measuring brain activity can include dividing the electrical brain wave patterns into several frequency bands. The common EEG frequencies include Delta, Theta, Alpha, Beta, and Gamma bands of brain activity. The amounts of activity in each EEG band provides information about brain state. FIG. 10 shows activity measured in each EEG band.

### Delta Band

The Delta band represents low frequency brain activity associated with resting states, deep concentration, or sleep. When activity is high (warmer colors) in this band, your brain is in a resting or drowsy state. When activity in this band is low (cooler colors) then your brain is more active and alert.

### Theta Band

The Theta band is associated with a relaxed state of mind that is most active when you are conscious, but not engaging in learning or a specific task. For example, theta brain wave activity is higher when attention is inwardly focused like during meditation. When your brain is actively engaged in learning, problem solving, or outwardly focused then theta activity is lower.

### Alpha Band

The Alpha band is associated with an active level of thought during which one is not actively processing information. Alpha consists of the everyday thoughts that flow through your mind when you are not concentrating. It is considered a learning state when you are alert but not actively processing.

### Beta Band

The Beta band is associated with active thought and information processing when you are focused on a task or outcome. High levels of Beta are believed to reflect a busy mind or actively engaged brain state, whereas lower levels of Beta reflect a calmer mind.

### Gamma Band

The Gamma band reflects a deeper focused brain state where attention is locked on a task, deep concentration, or problem solving. High gamma activity is also thought to reflect synchronous processing of multiple streams of information in the brain. Lower levels of gamma activity simply reflects that your brain is not locked into working on a specific task or solving a specific problem at that moment.

### Synergy Score

A Synergy map reflects the degree of similarity or levels of activity across your brain. The Synergy score is high as indicated by warmer colors when all your thoughts are in sync. A high Synergy score is ideal just prior to performing tasks or activities to achieve the best results.

### Serenity Score

A Serenity map reflects the level of efficiency in your brain activity. The brain is designed to be efficient. A high Serenity score indicated by warmer colors indicates that your brain is active and not necessarily in the most efficient state. A low Serenity score indicated by cooler colors means that you are in a resting state and your brain is performing tasks more efficiently. Low Serenity brain scores occur during an efficient performance or when you have a calm mind.

### Example 3: Mobile Application and Measurement of Brain Activity

A mobile application is used to monitor brain activity of subjects. The mobile application pairs to a subject's device. A subject logs onto the application using their subject ID, which does not contain any identifiable information. All data is securely stored remotely on a HIPAA compliant Amazon Web Services (AWS) server under an AWS Business Associate Addendum. The application is designed to acquire data regarding stress and anxiety using a modified version of the Short State Trait Anxiety Inventory (STAI-6) once per day. An example of a STAI-6 is shown in FIG. 11. The application collects data on mood using a modified version of the Russel Mood Circumplex twice daily. The application records information about sleep time and quality, fatigue, and drug use in the morning and evening using a modified version of the NIH Sleep Diary. Examples of sleep diary questions are shown in FIG. 20.

The application works with a custom engineered, at home EEG device (e.g., a CGX, LLC device) that records activity from the prefrontal cortex using a wearable, low-risk device. The application pairs automatically to the device using Bluetooth. The rechargeable, battery-powered, wearable EEG device uses disposable, biocompatible, snap biopotential sensor electrodes. Users can record resting state at any time they wish in one-minute epochs. The option to record resting state brain activity in the morning and evening is built into the questionnaires. Subjects are asked to record 1-minute epochs of resting state EEG activity in the morning or evening at least 4 times per week. Subjects can record resting state EEG activity as often as desired. An example questionnaire for recording brain activity is shown in FIG. 21. All data are stored with the subject ID and no other identifying information, such that only the study personnel can de-identify the data using the subject ID key which is kept with the PI and/or study coordinator.

Changes from baseline and placebo against active psilocybin on the symmetry and power of alpha, beta, delta, and gamma brainwave activity across prefrontal cortex brain regions are evaluated.

The risks of occurrence of skin irritation or headache is minimized through proper instructions of use documentation, training of human research subjects on proper study device use, and in the usage and design of biocompatible electrode materials for device construction.

### Example 4: Clinical Trial to Determine How Doses of Psilocybin Affect Brain Activity

### Study Objectives

The study determines how micro-doses and low-doses of psilocybin affect subcortical, thalamic, and cortical brain circuit plasticity in a manner that can predict improvements in emotional and cognitive function. The study characterizes the neurophysiological and psychological effects of micro- and low-dose psilocybin on: 1) brain activity using EEG; 2) on cognitive and emotional performance using psychophysical tasks; 3) on mood, anxiety, and sleep using clinical surveys; and 4) biochemical signaling using saliva analysis of cytokines and brain-derived neurotrophic factor.

The study is designed to begin identifying effective dose ranges using quantitative insights gained from EEG during real-time processing of sensory, emotional, and psychological stimuli. The study characterizes how micro- and low-dose psilocybin alter human stimulus-response functions using the approaches described, while subsequently tracking the duration of effects on mood, attention, social function, anxiety (stress), cytokine and neurotrophic factor signaling, and sleep.

By characterizing the influence of psilocybin on sensory processing in response to auditory stimuli, the study develops an objective, quantitative measure of hallucinogenic threshold using EEG of brain activity. More specifically, the study determines if auditory brainstem responses (ABR), paired pulse inhibition (PPI; i.e., P50 auditory suppression; sensory gating), auditory mismatch negativity (MNN), and auditory steady state responses (ASSR) are affected in a dose-response manner by micro- and low-dose psilocybin treatment. By sampling short-term plasticity using auditory stimuli in this manner the study assesses changes happening early in sensory processing streams at the level of the brainstem, higher up in thalamic relays, and even higher in the cortex at conscious levels of processing.

EEG signals have not been examined at low- or micro-doses, which have also shown to produce therapeutic benefits. Therefore, the study determines if EEG measures of auditory sensory evoked activity, cognitive control of attention and emotion can provide insights into characterizing therapeutically effective doses where hallucinogenic activation can be avoided in chronic dosing regimens.

### Study Design

The study is a within-groups, double-blinded, randomized controlled study. Each subject is scheduled to undergo four different low- and micro-dose treatments of psilocybin and a sham control (niacin) in a randomized order where one treatment occurs every 7-10 days as described below.

The four psilocybin treatment doses and one sham treatment to be used in this study as follows:
Low-dose HIGH 0.15 mg/kg (˜10 mg based on 70 kg subject weight);
Low-dose LOW 0.075 mg/kg (˜5 mg based on 70 kg subject weight);
Micro-dose HIGH 0.030 mg/kg (˜2 mg based on 70 kg subject weight);
Micro-dose LOW 0.015 mg/kg (˜1 mg based on 70 kg subject weight); and
Sham treatment consists of niacin (100 mg vitamin B3)

Psilocybin and niacin are acquired from Organix, Inc. (Waltham, MA). Organix, Inc., has supplied several investigator-initiated IND's for IRB-approved studies.

### Study Methods

The study implements a Clinical Trial Management and Electronic Data Capture (EDC) Software package from OpenClinica. This software is compliant with GCP, FDA, HIPAA, 21CFR Part 11, and other guidelines. The software provides 256-bit encryption technology for participant data protection, provides fully integrated tools for scheduling, administering, tracking, storing, and securing electronic consent (eConsent) and electronic participant reported outcomes (ePRO). The software also provides integrated solutions for participant flow management, participant monitoring and communication tools, participant scheduling, compliance monitoring, data backup, data analysis, data reporting, and data export capabilities. The study uses OpenClinica to administer electronic forms, surveys, and questionnaires, as well as electronic informed consent for all aspects of the study.

Subjects are assigned to random treatment dose schedules following informed consent and study enrollment. Each subject undergoes weekly treatment with one of four psilocybin doses and one placebo in a randomized order. Treatment dose schedules are randomly computed using a random number generator program with equal probabilities of drug dose assignment including placebo. The PI maintains the code in a secure location on OpenClinica not accessible until unblinding occurs at the end of the study. The PI, study coordinator, and study monitors, as well as subjects remain blind to the treatment assignment until unblinding following analyses. All instructions and materials provided to subjects are identical across the treatment.

The study achieves double blinding using unique 8 digit, random alpha numeric codes that correspond to the randomly generated dosing schedule. Once all data is collected, processed, and coded for statistical analyses the study is unblinded. Drugs are also blinded using an 8 digit alpha numeric code. Should there be an adverse event or medical emergency that requires unblinding for the subject then the PI or Co-PI unblinds the conditions for the subject without compromising the blind conditions of other subjects in the study.

Daily compliance monitoring is conducted throughout the study using EDC/Clinical Trial Management Software OpenClinica by study monitors and a mobile application. The mobile application can be the mobile application described in Example 3.

The application enables users to review history and maps, includes a profile view, and allows users to select between AM or PM surveys, as described below. The application uses a mobile device's camera for HRV spot-checking, and integrates nasal, box breathing, and microdose questions.

The application is the world's first database for monitoring the acute and chronic effects of psychedelics on brain activity, mood, and coping. As mentioned above, the application uses at home EEG devices for high quality brain mapping using proprietary EEG algorithms. The application connects patients and users to medical and recreational marijuana, as shown in FIGS. 18A-18B.

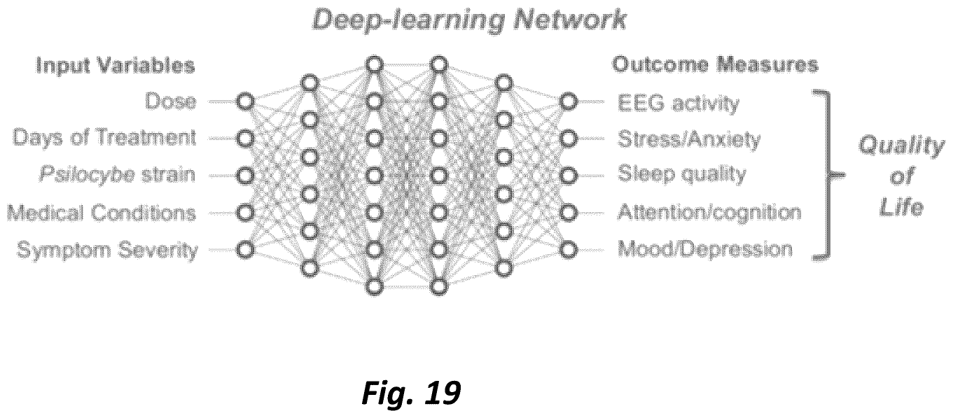

The application implements artificial intelligence and machine learning to lead psychedelic medicine, as shown in FIG. 19.

A data collection log in the EDC/Clinical Trial Management Software is used to ensure all scales and study measures are acquired for all subjects at appropriate time points and phases of the study. Study personnel conduct weekly data integrity checks to ensure the data formats and storage for all incoming data is in the proper formats, does not contain missing data, and is securely stored for later analyses.

Additional study monitoring functions are performed by qualified, experienced and independent clinical monitor(s) in compliance with applicable U.S. regulations (21 CFR Part 312 [Investigational New Drug Application], 21 CFR Part 50 [Protection of Human Subjects] and 21 CFR Part 56 [Institutional Review Boards]), Good Clinical Practice, ISO 14155:2011 and recommendations guiding physicians in biomedical research involving human subjects adopted by the 18th World Medical Assembly, Helsinki, Finland, 1964. The monitor(s) oversee progress of the investigation and work with study site personnel to ensure adherence to the study protocol and informed patient consent obligations.

The Study End Point is the completion of the 3-month follow-up surveys following treatment, withdrawal for any medical reason, or voluntary participant withdrawal from the study. Participants may withdrawal from the study at any time for any reason by notifying the PI, Co-PI, or Study Coordinator. If at any time during the study the subject's medical status changes he/she should notify the PI or Study Coordinator so a determination of potential withdraw must be made. If a subject fails to meet the exclusion criteria for a medical reason during the study then he/she should be immediately withdrawn from the study and paid subject payment. If a subject misses a treatment then he/she should resume the treatment schedule the following week if possible. Participants may be withdrawn if they miss more than one treatment over a six-week period. If any subject is withdrawn from the study the data acquired up through the withdrawal event may be included in the study with the remainder of the data points contributed to the study classified and treated as missing values. The survivability of the data is determined on a case-by-case basis. Data should not be considered for survivability unless both at least three treatment days were recorded. If subjects withdraw from the study permission is obtained to continue including their data in the study for survivability to be included in final analyses and report.

### Study Outcome Measures: Primary Outcome Measures

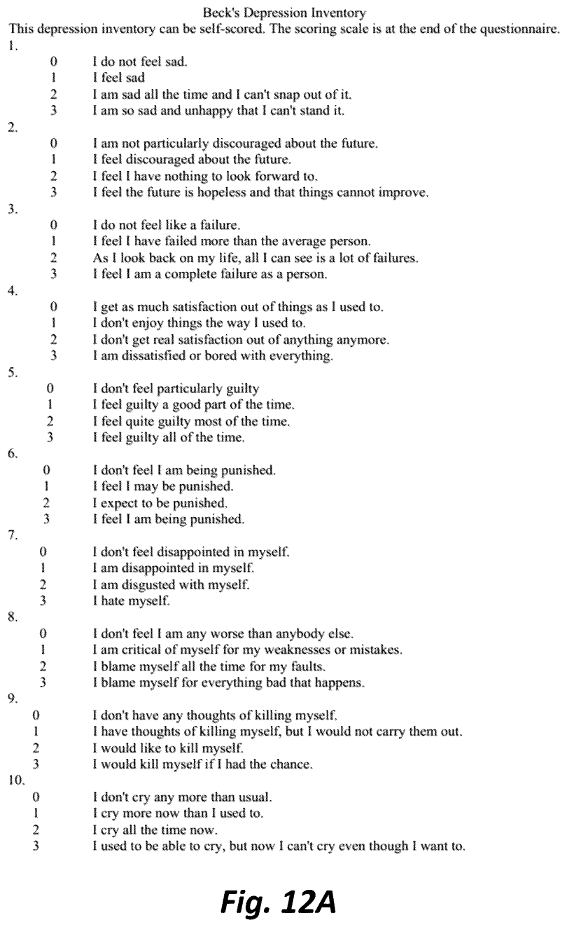

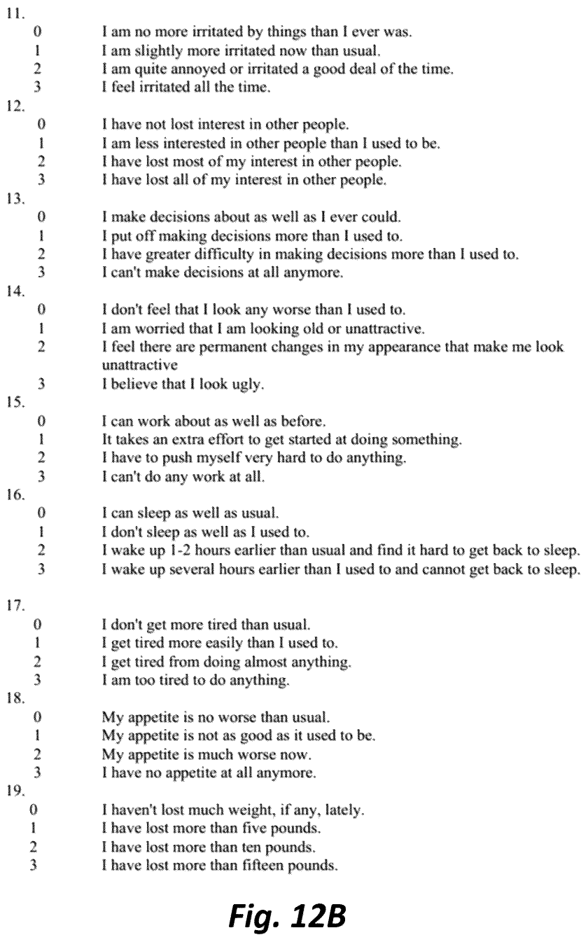

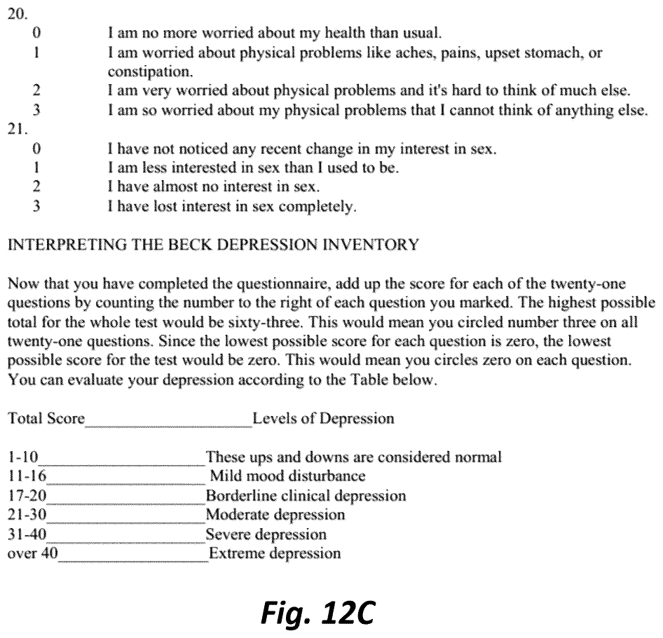

Change from baseline and placebo control in depression severity using the Beck Depression Inventory (BDI). The Beck Depression Inventory (BDI) is a 21-item, self-report rating inventory that measures characteristic attitudes and symptoms of depression. The BDI is one of the most widely used instruments to measure depression severity. It is composed of items relating to symptoms of depression such as hopelessness and irritability, cognitions such as guilt or feelings of being punished, as well as physical symptoms such as fatigue, weight loss, and lack of interest in sex. A BDI greater than or equal to 20 indicates moderate depression and is used as an inclusion-criteria for enrollment in the study. The BDI is administered at the beginning of the study (Office Visit #1, baseline) and once weekly (before each treatment) thereafter until one week following the last treatment. Changes in BDI scores are calculated and compared to baseline, time in the study, across treatment dose. FIGS. 12A-12C illustrate an example BDI.

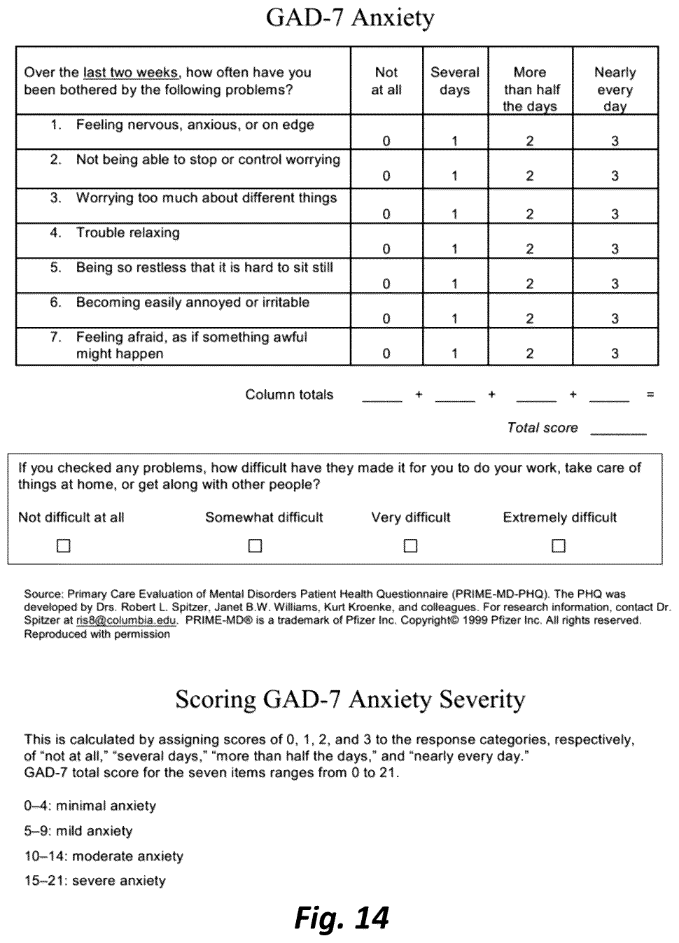

Changes from baseline and placebo treatment in anxiety symptoms using the GAD-7. The Generalized Anxiety Disorder (GAD-7) questionnaire is a seven-item, self-report anxiety questionnaire designed to assess the patient's health status. The GAD-7 inquires about the degree to which the patient has been bothered by feeling nervous, anxious or on edge, not being able to stop or control worrying, worrying too much about different things, having trouble relaxing, being so restless that it is hard to sit still, becoming easily annoyed or irritable and feeling afraid as if something might happen. A GAD-7 score greater than or equal to 10 reflects moderate anxiety and is used as an inclusion-criteria for enrollment in the study. The GAD-7 is administered at the beginning of the study (Office Visit #1, baseline) and once weekly (before each treatment) thereafter until one week following the last treatment. Changes in GAD-7 scores are calculated and compared to baseline, time in the study, across treatment dose. An example GAD-7 questionnaire is shown in FIG. 14.

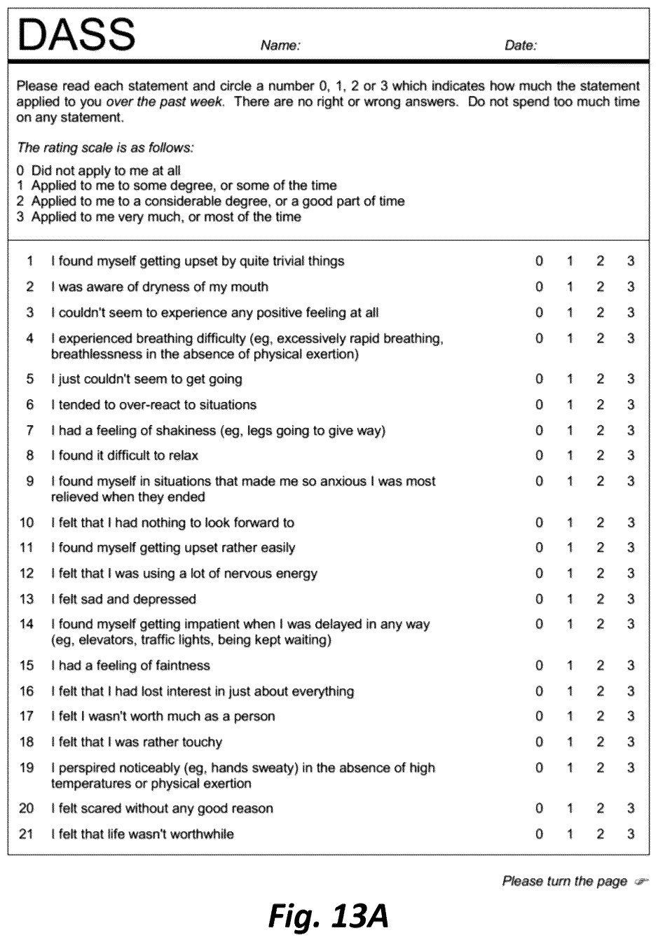

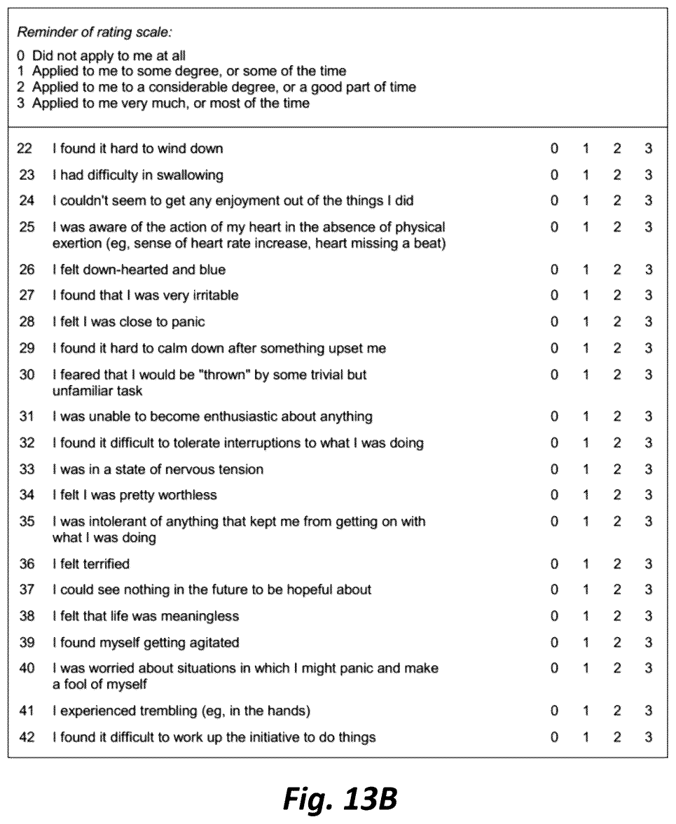

Changes from baseline and placebo treatment in depression, anxiety, and stress severity using the DASS-42. The Depression Anxiety Stress Scale (DASS) is a 42-item self-report instrument designed to assess depression, anxiety, and stress, indexed to the past week. Each subscale is comprised of 14-items. Each item is assessed on a 4-point scale, ranging from 0-did not apply to me at all to 3-applied to me very much or most of the time. The DASS has been shown to have high internal consistency. The DASS is administered at the beginning of the study (Office Visit #1, baseline) and once weekly (before each treatment) thereafter. A DASS depression score greater than or equal to 14 indicates moderate depression and is used as an inclusion-criteria for enrollment in the study. A DASS anxiety score greater than or equal to 10 indicates moderate anxiety and is used as an inclusion-criteria for enrollment in the study. Subjects are asked to provide DASS responses based on their past week. Changes in DASS scores are calculated and compared to baseline, time in the study, across treatment dose. An example DASS is shown in FIGS. 13A-13B.

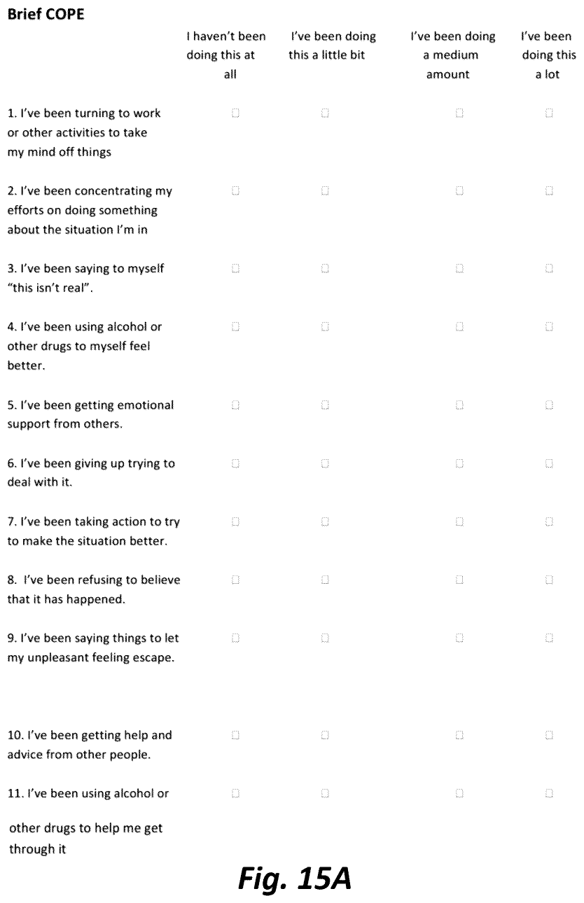

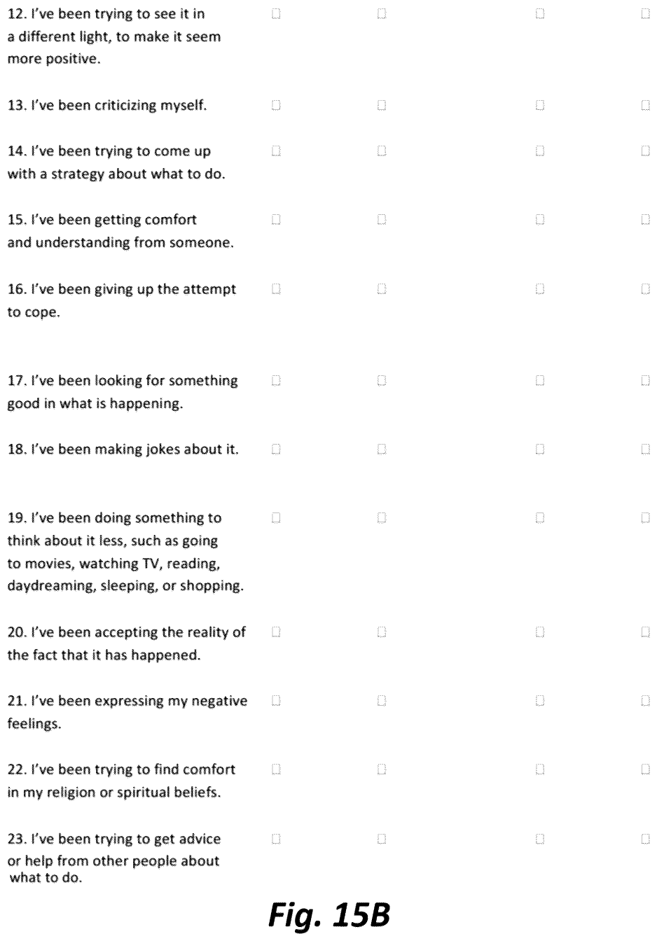

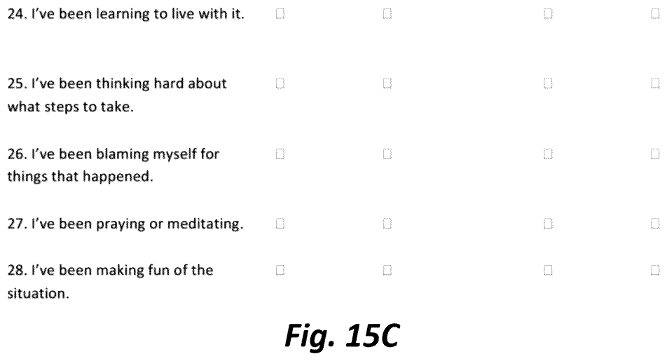

Changes in coping strategies and coping approaches from baseline and placebo treatment using the brief COPE scale. The Brief-COPE is a 28 item self-report questionnaire designed to measure effective and ineffective ways to cope with stressful life events. “Coping” is defined broadly as an effort used to minimize distress associated with negative life experiences. The Brief-COPE scale measures coping across two major domains: approach and avoidance coping. The test has been shown to be valid and have high specificity. The brief-COPE scale is administered at the beginning of the study (Office Visit #1, baseline) and once weekly (before each treatment) thereafter. Subjects are asked to provide brief-COPE scale responses based on their past week. Changes in brief-COPE scale scores are calculated and compared to baseline, time in the study, across treatment dose. An example Brief-COPE is shown in FIGS. 15A-15C.

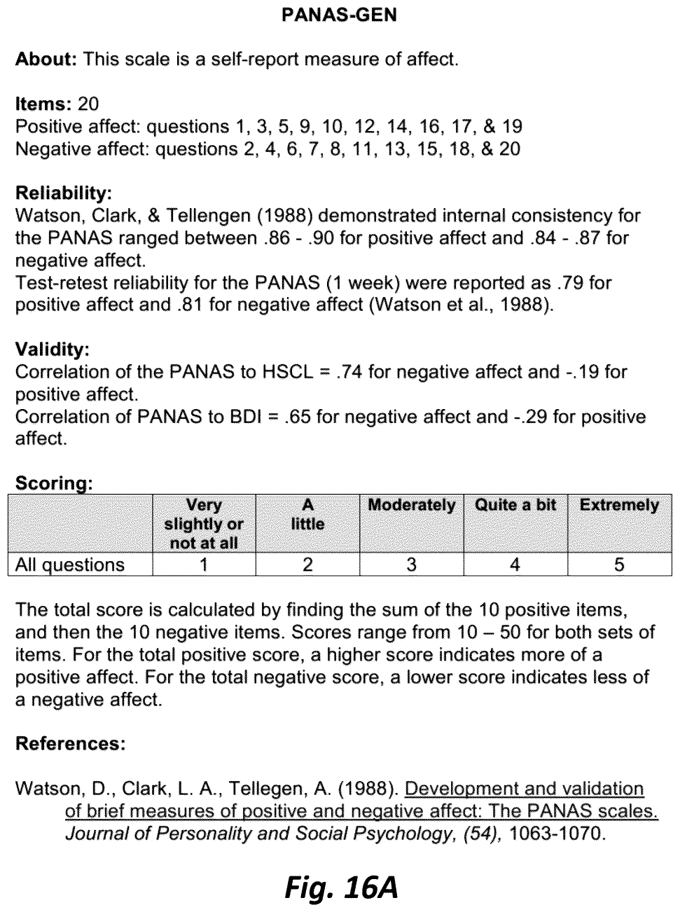

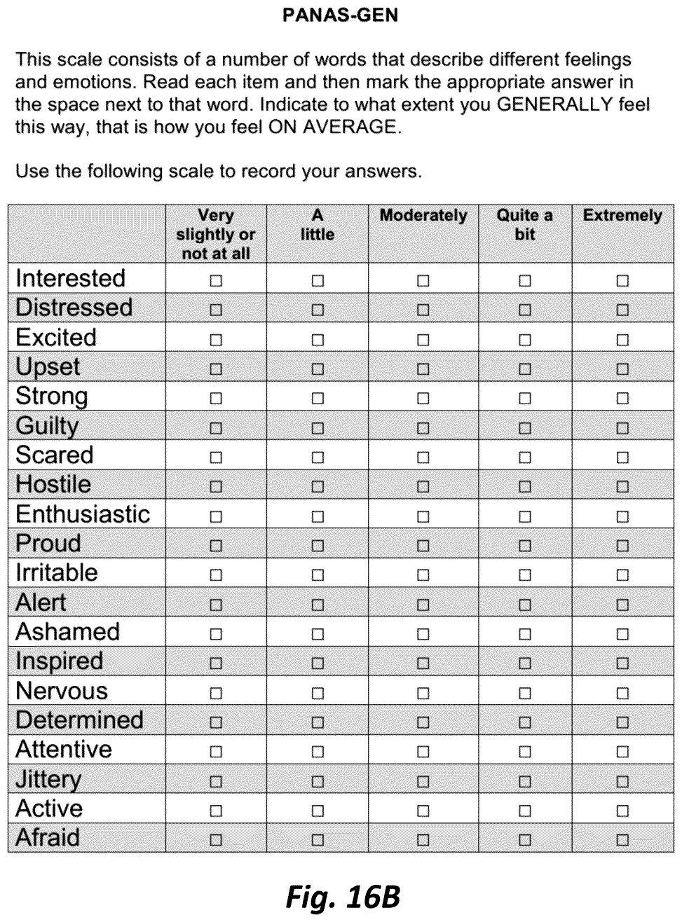

Changes in mood and affect from baseline and placebo treatment using the PANAS. The Positive and Negative Affect Schedule (PANAS) is a self-report measure of weekly affectivity, with positive affect and negative affects subscale each being 10 items. The scales have demonstrated high internal consistency, are largely uncorrelated, and remain stable at appropriate levels over a 2-month time period. The scale is re-indexed to assess affect over the prior week and is administered at baseline (Office Visit #1) and weekly thereafter (before each treatment). Changes in PANAS scores are calculated and compared to baseline, time in the study, across treatment dose. An example PANAS is shown in FIGS. 16A-16B.

### Study Outcome Measures: Secondary Outcome Measures

Change in amplitude and spectral power of EEG potentials during P50 paired click auditory suppression tasks. Changes in the amplitudes and power of EEG auditory potentials recorded to paired stimuli provide insights about brain activity occurring at sub-thalamic levels. These changes are believed to reflect sensory gating processes. The study examines the amplitude and spectral power of EEG potentials from frontal, temporal, and parietal EEG sites using 10-20 configuration. The study compares changes in the amplitude and power of EEG potentials recorded during placebo treatment with active psilocybin treatments. Changes in amplitudes of P50 are correlated to depression, anxiety, and mood survey data.

Change in amplitude and spectral power of EEG potentials during auditory Mismatch Negativity (MMN). Changes in the amplitudes and power of EEG auditory potentials recorded to oddballs provide insights about brain activity occurring at sub-thalamic and cortical levels. These changes are believed to reflects changes in bottom-up sensory processing, as well as top-down cognitive (attentive) control of sensory responses. The study examines the amplitude and spectral power of EEG potentials from frontal, temporal, and parietal EEG sites using 10-20 configuration. The study compares changes in the amplitude and power of EEG potentials recorded during placebo treatment with active psilocybin treatments. Changes in amplitudes of MMN potentials are correlated to depression, anxiety, and mood survey data.

Change in amplitude and spectral power of EEG Auditory Steady State Responses (ASSR). Changes in the amplitudes and power of EEG auditory potentials recorded to steady state stimuli provide insights about brain activity occurring at sub-thalamic and cortical levels. These changes are believed to reflects sensory adaptation processes. The study examines the amplitude and spectral power of EEG potentials from frontal, temporal, and parietal EEG sites using 10-20 configuration. The study compares changes in the amplitude and power of EEG ASSR potentials recorded during placebo treatment with active psilocybin treatments. Changes in amplitudes of ASSR EEG signals are correlated to depression, anxiety, and mood survey data.

Change in the accuracy and reaction times on the Emotional Flanker Task. The study implements a modified Erikson Flanker Task administered on a computer. Neutral or angry faces are used as a background distractor on foreground Flanker test stimuli. Subjects respond to congruent, incongruent, and neutral Flanker stimuli on neutral and angry faces. Changes in reaction time and accuracy reflect several aspects of impulsivity, cognitive control, emotional sensitivity, and attention performance. Changes in reaction time and accuracy are compared for placebo control against the different doses of psilocybin.

Change in the accuracy and reaction times on the Continuous Performance Test (CPT). The study implements the Connor's CPT administered on a computer. Subjects respond to congruent, incongruent, and neutral stimuli using target cues. Changes in reaction time and accuracy reflect several aspects of impulsivity, cognitive control, emotional sensitivity, and attention performance. Changes in reaction time and accuracy are compared for placebo control against the different doses of psilocybin.

Change in the amplitude and spectral power of EEG error potentials during emotional flanker and CPT tasks. Detected by EEG during some psychophysical tasks like the CPT and Flanker Task, a potential emerges when errors are made that occurs within 100 milliseconds of stimulus response. The brain automatically processes whether an error is made and this is believed to be the recognition of conflict processing in the brain similar to that which occurs during MMN or oddball tasks. The study evaluates changes in the amplitude and spectral makeup of EEG error potentials detected during CPT and Flanker Tasks for placebo treatment against active psilocybin.

Changes in the levels of daily anxiety from baseline and placebo using daily STAT-6 surveys. The State Trait Anxiety Inventory is a 6-item, self-report survey that is designed to assess anxiety levels. Subjects use the application to log daily STAI-6 responses. An example of STAI-6 questions are shown in FIG. 21. The study begins recording STAI-6 data one week prior to the first treatment and continues daily recordings until one week following the last treatment. Data are compared from baseline against week-to-week and day-to-day changes as a function of psilocybin dose or sham. An example of a STAI-6 survey is shown in FIG. 11.

Changes in sleep quality and efficiency from baseline and placebo using a daily Sleep Diary. Each morning during the study subjects use an application to respond to a sleep diary assessing sleep latency, sleep quality, sleep time, wake after sleep onset, number of awakenings, and morning fatigue. Examples of sleep diary questions are shown in FIG. 20. The diary also includes entries to log any drugs or naps taken throughout the day. Subjective sleep diaries have been shown to be a reliable measure of sleep behavior and they have been validated against EEG polysomnography. The study begins recording sleep diary data one week prior to the first treatment and continues daily recordings until one week following the last treatment. Data are compared from baseline against week-to-week and day-to-day changes as a function of psilocybin dose or sham. Changes from baseline or sham values are computed for the measures captured in the sleep diary as described above.

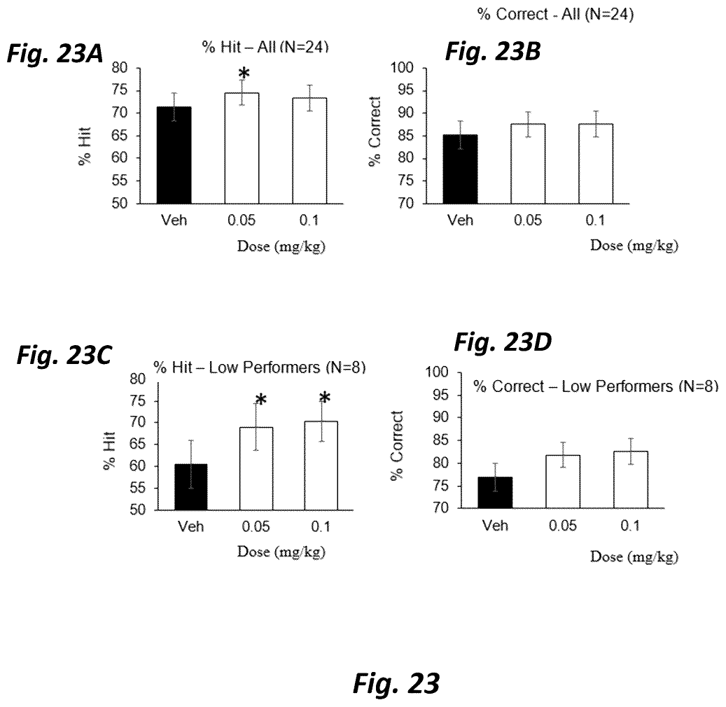

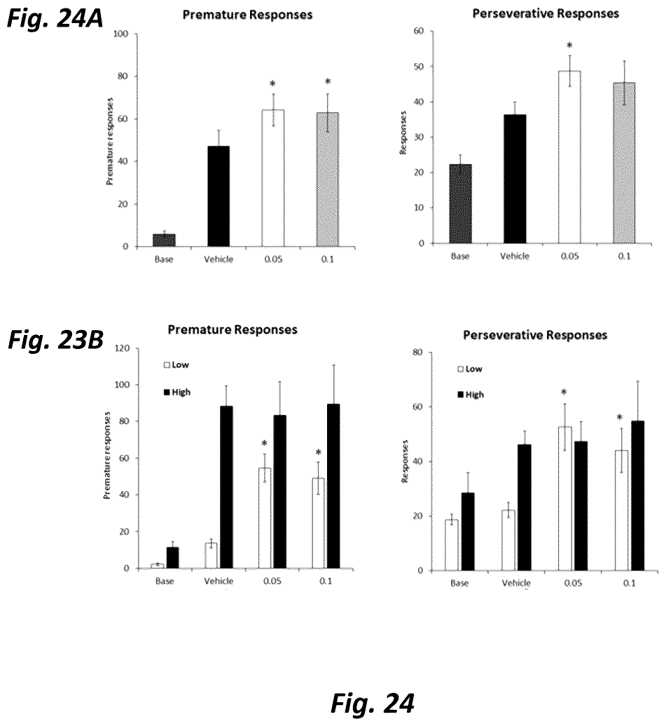

Changes in the symmetry and power of daily brain activity during resting state EEG. Resting state EEG is believed to reflect activity of the default mode network circuitry, which tends to be hyperactive in depressed and anxious patients. Using wearable EEG headbands from CGX Systems (Cognionics), subjects record resting state brain activity for 30 seconds at least once, and up to four times daily through the application. Examples of recording brain activity in the morning and evening in the application are shown in FIG. 22 and FIG. 23. The study begins recording daily EEG resting state activity one week prior to the first treatment and lasting through one week following the last treatment. The study evaluates changes from baseline and placebo against active psilocybin on the symmetry and power of alpha, beta, delta, and gamma brainwave activity across prefrontal cortex brain regions.

Changes in Daily Mood. The study records changes from day-to-day in mood using a modified Mood Circumplex to assess mood valence (positive or negative) and mood arousal (high or low). The Mood Circumplex is delivered daily using the application. Changes in self-reported mood states are recorded beginning one week before the first treatment and last through one week after the last treatment. Changes from baseline or sham values are computed for the measures captured by the Mood Circumplex.

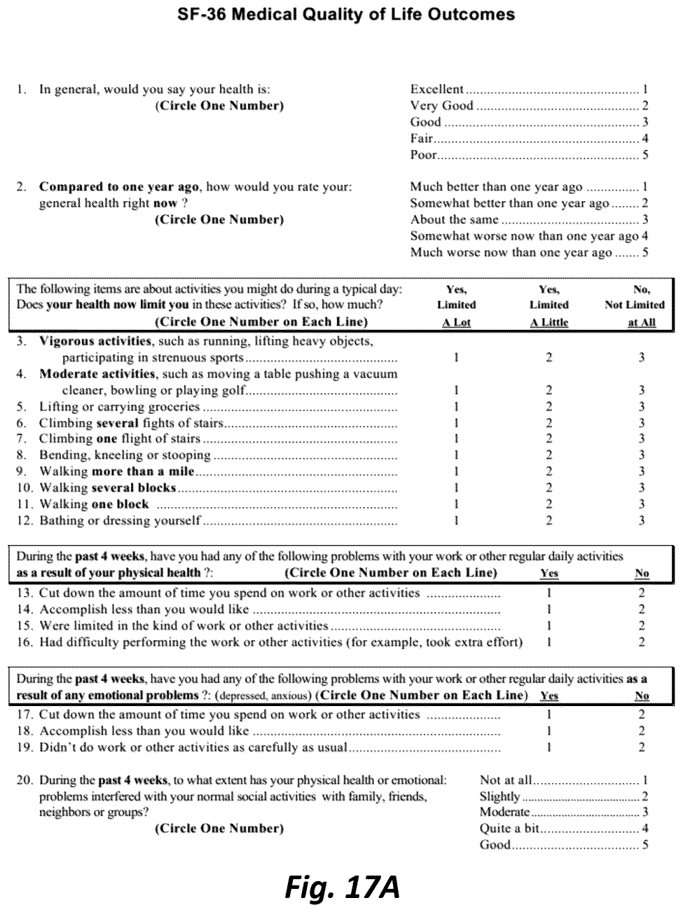

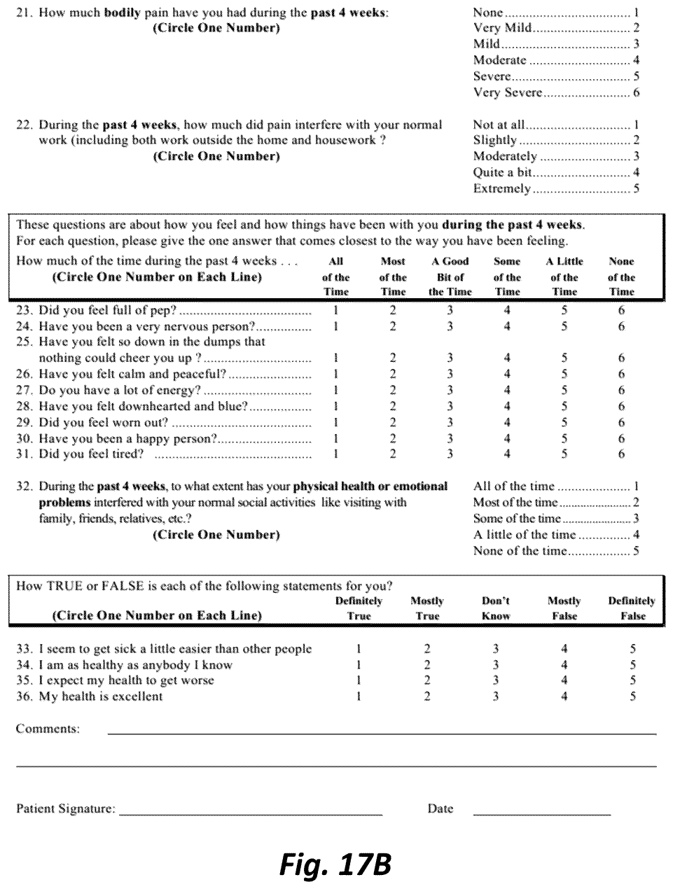

Changes in the quality of life: Medical Quality of Life Outcomes Study 36-Item Short Form Health Survey (SF-36) and Physical Component Summary (PCS) and Mental Component Summary (MCS) scores. The SF-36 is a subject-reported outcome measure evaluating participant's health status. It comprises 36 items covering 8 domains: physical functioning, role physical, role emotional, bodily pain, vitality, social functioning, mental health, and general health. Items are answered on Likert scales of varying lengths. The 8 domains are regrouped into the PCS and MCS scores. The summary scores range from 0 to 100, with higher scores indicating better levels of function and/or better health. In this study, the SF-36 acute version are used, which has a 1 week recall period. The SF-36 is administered at Office Visit #1 and weekly thereafter (before each treatment) through one week past the last treatment. The SF-36 is also administered 1 and 3 months post-treatment follow-up. An example SF-36 is shown in FIGS. 17A-17B.

Changes in the concentration of salivary cytokines and salivary brain-derived neurotrophic factors are also recorded.

### Primary Safety Measures

Adverse Events: Treatment number of subjects experiencing adverse events (Time Frame: length of study).

Adverse Reactions: Treatment versus sham incidence and severity of adverse reactions measured on categorical and continuous scales (Time Frame: length of study).

Comfort in response to active psilocybin treatment versus sham level of treatment comfort. The study uses a custom survey of 73 items to evaluate the number and level of emotional and physical side effects experienced from placebo and active treatment groups. The study also uses the 5D Altered state of Consciousness Scale (5D-ACS) to evaluate psychedelic activity and comfort. The scales are administered at the end of each treatment day prior to a subject being released. The study compares the levels of emotional, physical, and psychological comfort between placebo and active treatment groups. Questions in the 5D-ACS can include: I had the feeling everything around me was somehow unreal; I felt as though I were floating; The boundary between myself and my surroundings seemed to blur; I felt totally free and released from all responsibilities; I had the feeling that I had been transferred to another world; It seemed to me that there were no more conflicts and contradictions in the world; It seemed to me as though I did not have a body anymore; I felt very happy and content for no outward reason; I could have sat for hours looking at something; I was completely indifferent toward everything; I experienced past, present and future as a oneness; It seemed to me that my environment and I were one; It seemed to me that I was dreaming; I had difficulty in distinguishing important from unimportant things; My thinking was constantly being interrupted by insignificant thoughts; My own feeling seemed strange to me, as though they did not belong to me; I felt tormented without knowing exactly why; I felt like a robot; My surroundings seemed peculiarly strange to me; I felt threatened without realizing by what; I had the feeling that I no longer had a will of my own; I was afraid without being able to say exactly why; I felt like a marionette; Everything around me was happening so fast that I no longer could follow what was really going on; I stayed frozen in a very unnatural position for quite a long time; I had difficulty making even the smallest decision; I felt as though I were paralyzed; Things around me appeared distorted to me; Time passed more slowly than usual; I was not able to complete a thought: my thoughts repeatedly become disconnected; I felt isolated from everything and everyone; It seemed to me that I no longer had any feelings; It seemed to me as though there were an invisible wall between me and my surroundings; I observed myself as though I were a stranger; I felt a total emptiness in my head; So many thoughts and feelings assailed me at once that I became confused; I saw lights or flashes of light in total darkness or with closed eyes; I saw scenes rolling by like in a file in total darkness or with my eyes closed; Objects around me engaged me emotionally much more than usual; Things around me had a new strange meaning for me; I saw colors before me in total darkness or with closed eyes; I saw things that I know were not real; I saw regular patterns in complete darkness or with closed eyes; Something occurred to me and I did not know whether I had dreamt or actually experienced it; I had dreamt or actually experienced it; I saw strange things, which I now know were not real; Everyday things gained a special meaning for me; Sounds seemed to influence what I saw; The colors of the things I saw were changed by sounds and noises; Sounds and noises sounded different than usual; Time passed faster than usual; I simply could not get rid of some unimportant thought; I became conscious of another “I” being hidden behind my usual “I”; The ground I was standing on seemed to be swaying; My ears were buzzing; I could not remember what had happened two hours earlier; I had the vague feeling that something important would happen to me; Parts of my body seemed no longer to belong to me; I had the feeling my limbs were larger than usual; I was convinced that I had experienced the same situation before; Things around me had a different smell than usual; I was tired and exhausted but at the same time wide awake; It seemed that I had once dreamed what I was experiencing; I perceived peculiar relationships between widely diverging matters; I had trouble distinguishing between what I imagined and what I really experienced; I no longer knew where I actually was; I had the feeling I could think faster or more clearly than usual; So many thoughts came to my mind that I no longer was able to organize them properly; I was too wide awake and too sensitive; I had the impression that everything occurring around me was related to me; I had the feeling that I could no longer control the movements of my body; and I felt influenced by electric currents, rays, or hypnosis.

### Statistical Methods

The study procedures and protocols include a separate Statistical Analysis plan that serves as a standalone document from the IRB document, but which is cross referenced by the IRB document. This allows the study to make changes to planned and ongoing statistical analyses plans without making modifications to the IRB document, which require IRB re-review and approval. No key elements or major comparisons are changed in the statistical plan, but rather the specific tests, normalization procedures, score conversion, and data organization strategies are updated and edited as needed. This is a standard practice given the statistical analysis plans implemented often change throughout the course of a study and IRB-approved study period. Based on an estimated Effect Size of 0.3 (alpha=0.05) the study includes up to 30 subjects completing all treatments in the study for a Power (1-beta)=0.9. This is a sufficiently powered investigational study. The enrollment rate is anticipated to be about 5-7% of candidates contacted. Thus, screening 500-600 subjects to enroll and complete 30 subjects in the trial is anticipated. An attrition rate of about 20% is anticipated. Thus, the need to enroll up to 40 subjects in order to gain data from 30 complete subjects is anticipated. Once data is collected from 30 subjects, recruitment and enrollment is stopped.

The study uses a combination of statistical methods including Bayesian Methods, Mixed Model Repeated Measures (MMRM), repeated measures ANOVA and ANCOVA, and regression analyses to evaluate psilocybin and sham treatment effects on Primary and Secondary Endpoints related to sensory processing plasticity, cognitive-emotional plasticity, mood, stress, and sleep in subjects struggling with moderate depression. The study uses repeated measures ANOVA and Chi-Square tests to evaluate Primary Safety Endpoints.

The study contains several independent and dependent variables which are expected to covary across one another to produce different effects on the Primary and Secondary endpoints. To account for these covariances, the study relies primarily on MMRM procedures where possible. The repeated measures study is designed to monitor within-subject and between-dose effects compared to baseline measures. The dependent variables, Primary and Secondary Outcome Measures are described above with basic statistical analysis procedures used for each. The study additionally relies on the subject matter expertise of a study biostatistician to assist us in further analyses.

The study analyzes data from all protocol-compliant subjects. The study determines protocol-compliance based on tracking data in OpenClinica. Further, subjects should complete all treatments to complete the study, as well as complete all surveys to be deemed protocol compliant. The study also uses data from subjects who fail to meet protocol-compliance, but who undergo baseline measures and at least two treatments of sham or psilocybin (determined after de-blinding).

Data from subjects in the active treatment groups (psilocybin) are compared against data obtained when experiencing the sham treatment as the comparator control. An additional comparator control is the data obtained during baseline before treatments begin at enrollment or from week to week before each treatment as described.

### Study Drugs: Psilocybin and Niacin

Psilocybin and niacin are obtained under an Investigator-initiated IND from Organix, Inc's Investigational Supply Program.

Psilocybin is controlled by Dr. Ruchir Patel in a double-locked facility with a drug log. Psilocybin or niacin are mixed based on morning body weight of the participant. The treatment compound is prepared the morning of treatments using an alpha-numeric code to remain blinded by Dr. Patel. Briefly, 5 milligram capsules are dissolved in 5 ml sterile water to make a 1 mg/ml solution assisted by heat and sonication in a water bath. Subjects are administered the treatment solution in a final diluted volume of 20 milliliters for all treatments. Use of all drugs is recorded in a log with the date and corresponding patient Study ID.

### Subject Tasks

The study involves eight office Study Visits as described below. The study involves eight office Study Visits separated by 7-10 days each as described below.
Week 1, Study Visit #1: Screening, EKG, Informed Consent, Enrollment, and Education Session 1 (baseline); duration 2-4 hours
Week 2, Study Visit #2: Education and Training Session 2; duration 2-4 hours
Week 3, Study Visit #3— Treatment 1 (7-10 days from Study Visit #1); duration up to 8 hours
Week 4, Study Visit #4— Treatment 2 (7-10 days from Treatment 1/Study Visit #3); duration up to 8 hours
Week 5, Study Visit #5— Treatment 3 (7-10 days from Treatment 2/Study Visit #4); duration up to 8 hours
Week 6, Study Visit #6— Treatment 4 (7-10 days from Treatment 3/Study Visit #5); duration up to 8 hours
Week 7, Study Visit #7— Treatment 5 (7-10 days from Treatment 4/Study Visit #6); duration up to 8 hours
Week 8, Study Visit #8— Study Exit (7-10 days from Treatment 5/Study Visit #7); duration up to 2 hours

Office Visit 1: Screening and Education. Upon being identified as a potentially eligible candidate for the study, candidates are contacted to schedule an office visit to undergo additional medical and psychological screening. The study is described in detail to subjects at this point. If subjects meet the screening criteria and are a suitable candidate for the study, an EKG is conducted. If the EKG returns no abnormalities as scored by a qualified physician or RN then the subject is enrolled in the study upon providing informed consent. Following informed consent and providing HIPAA authorization, the subject is enrolled in the study. The subject is provided with information and educational materials regarding the study and the use of psilocybin in mental health research. Subjects fill out surveys and schedule their second office visit.

Office Visit 2: Education and Training. On the second office visit, the educational materials are reviewed with subjects and answers to any questions they may have regarding the study are provided. Time is spent demonstrating the treatment procedures and protocol with the subject so that they are prepared for treatment day. Time is spent showing subjects the EEG recording devices and subjects are allowed to practice tasks, again so they become comfortable with the treatment environment, study personnel, and procedures.

Office Visits 3-7: Treatment Days 1-5. Enrolled subjects participate in 5 different treatments. Four of these are different doses of psilocybin and one is a placebo. The procedures for each of the treatment days are the same and as follows.
The night before each treatment, subjects are instructed to avoid food after midnight. Subjects may have juice, water, or soda, but should avoid caffeinated beverages.
On each Treatment day subjects are asked to ensure they have a ride home from the clinic. Subjects should arrive at the clinic by 8 am on the morning of the scheduled treatment and not plan to leave before 5 pm that day.
On each treatment day subjects are asked to answer some surveys (PANAS, BDI, DASS, Brief COPE, and GAD-7) in OpenClinica before treatment. Subjects are then given an oral dose of randomly assigned active psilocybin or placebo by mouth based on their body weight.
About 1.5 hours after treatment, subjects start undergoing EEG monitoring of brain activity while listening to auditory tones or performing computerized emotional and cognitive tasks as described. These tasks last about two hours.
Following EEG testing and cognitive tasks, subjects are allowed to remain in the clinic for the rest of the day where they can eat, drink, watch TV, use the internet, etc., at their leisure until permitted to leave around 5 pm or are cleared to leave by a physician. If patients are not cleared to leave, they can remain in the sleep clinic under observation overnight as necessary.
The day following treatment, someone from the research staff calls or sends an electronic communication to subjects to check-in with them.
Subjects repeat the treatment procedure for a total of five times separated by 7-10 days each.

The study implements the use of several screening tools, surveys, and assessments. These surveys and assessments are administered using the OpenClinica EDC software as described above or through other means as described below. The section below provides a brief description of each screening or assessment tool used in the study. Electronic document forms of each instrument is provided with the IRB protocol documents.

The study uses a brief medical screening questionnaire to evaluate whether subjects are medically stable and qualified for enrollment in the study based on the inclusion and exclusion criteria.

The consent form includes the inclusion and exclusion list. However, an Inclusion and Exclusion checklist is completed by subjects in assistance with study personnel prior to the subject providing informed consent for enrollment in the study. The Inclusion and Exclusion Checklist is administered one time at Study Visit #1 after pre-screening and prior to informed consent. The Inclusion and Exclusion Checklist is included with the IRB Protocol Study Documents.

A brief demographics form is used to collect age, gender, race, ethnicity, height, weight, educational level, family income level, and profession. No identifiable data is collected and data is linked to the participant Subject ID upon informed consent and study enrollment. The demographic survey is administered one time at Study Visit #1 after pre-screening, screening, and informed consent upon study enrollment. Electronic document forms of each are provided with the IRB protocol documents.

During Office Visit #1, a routine EKG (ECG) is administered which is scored by a physician, RN, or other qualified clinical professional. If the subject's EKG is returned with no abnormalities detected then the subject is eligible for enrollment in the study. If the EKG is abnormal however, the subject is not eligible for the study and should be excluded due to an irregular heart rhythm.

Upon informed consent and study enrollment, subjects respond to several survey instruments in the OpenClinica environment to establish baseline. Each subject completes the following surveys by the end of Office Visit #1: BDI, GAD-7, DASS, PANAS, and Brief-COPE. Details regarding these surveys are included above.

After informed consent and study enrollment, subjects undergo a training that lasts up to 4 hours. The first component of the education occurs at Office Visit #1 where subjects are provided with an information study packet containing information regarding psilocybin research in mental health. Subjects also watch a short video describing the research procedures and protocols. Subjects meet with research personnel, who discuss and describe all procedures that occur. During office visit #2, subjects continue acclimating to the study environment. The study site has rooms dedicated for treatment, EEG physiology, and psychophysical testing. These rooms are private, calm, and quiet environments setup like an apartment with access to TV, bathroom, bed, couch, food, and drink. The research personnel walk subjects through the entire treatment and testing procedure. Subjects are allowed to try on EEG headcaps and undergo mock experiments for training on the auditory listening tests (P50, ASSR, MMN) and psychophysical tasks (Emotional Flanker and CPT). This provides subjects with ample opportunity to understand exactly the processes in place before their first treatment. During this training opportunity, subjects continue to be interviewed by research personnel to ensure they are comfortable with the environment and procedures and to help ensure no psychiatric disqualifiers are present.

Immediately following treatment in the clinic, subjects begin being setup for EEG recordings. Approximately 1.5 hours after ingestion of the treatment dose, subjects undergo a series of three EEG auditory tasks including: P50 suppression, Mismatch negativity (MMN), and Auditory Steady State Response (ASSR). 20 channel and 32 channel EED headcaps are used throughout the study to record brain activity in response to auditory stimuli. Briefly, the P50 task involves the subject listening to 120 paired auditory clicks (1 millisecond each) occurring 500 milliseconds apart. Each pairing occurs 10 seconds apart. The estimated time to complete the passive P50 task is 20 min. The ASSR task requires subjects to listen to a series of tones using a 500 or 1000 Hz carrier frequency modulated at 20 or 40 Hz for 1 second each. Each 1 second tone is separated by a three second inter-stimulus interval. A total of 100 tones at 20 and 40 Hz each are delivered. The ASSR task is expected to last 15 minutes. The MMN task requires subjects to listen to 500 tones (1 millisecond) spaced 1 second apart. There are 400 tones at a standard frequency (750 Hz) and 100 randomly spaced odd-ball tones (1500 Hz). The MMN task is expected to take 10 minutes. At the end of the psychophysical tasks described below, subjects undergo a final resting state period of EEG collection where they lay down with their eyes closed for 10 minutes. EEG recordings also include other biosensors for recording heart rate activity during testing.

Following EEG assessments as described above, subjects undergo a series of computerized cognitive attention and emotional tasks while recording EEG. Subjects first complete an Emotional Flanker Test that uses neutral faces or an angry face as a background distractor on classic flanker stimuli assessing congruent, incongruent, and neutral conditions. A stimulus from each condition is presented 50 times and a total of 250 stimuli are presented over 10 minutes. Next, subjects take a Conner's Continuous Performance Test that lasts 14 minutes to assess attention and cognitive control.

Every week prior to treatment, subjects fill out weekly surveys assessing mood, depression, anxiety, and stress. Each subject completes the following surveys at least every 10 days and before every single treatment in the clinic: BDI, GAD-7, DASS, PANAS, and Brief-COPE. Details regarding these surveys are included above.

In between treatment days, subjects fill out daily surveys using the application. These surveys include the STAI-6, a Sleep Diary, and Mood Circumplex as described above. Subjects also use the application to record resting state brain activity at least once and up to four times per day for a 30 second period. Subjects spend no more than 5-7 minutes interacting with the application each day to complete these surveys and tasks.

Multiple outcome metrics are calculated using data collected from at-home, wearable EEG devices. Wearable EEG headband devices are used from CGX. Subjects wear the devices to record at least one, but up to four 30-second epochs of resting state brain activity with their eyes closed each day. This procedure takes less than 5 minutes to complete.

The DSM-V provides diagnostic criteria for depression. Several of the screening and diagnostic tools used in this study are also used in the clinical and research diagnosis of depression and anxiety. The BDI, GAD-7, DASS, PANAS, Brief-COPE, and STAI-6 are used by mental health professionals addressing psychological outcomes related to depression, anxiety, and stress disorders. The Flanker Task and CPT used in the study are less specifically used as diagnostic tools in the assessment of depression and anxiety, but are used in psychological research to evaluate cognitive, attention, and emotional performance. The auditory EEG tasks (P50, MMN, and ASSR) are not specifically used to evaluate depression or anxiety, but are used in psychological research to help understand the brain's processing of sensory cues in normal subjects and those suffering from psychological disorders.

The treatment of depression typically includes the use of pharmaceuticals or cognitive behavioral therapy. The type of psilocybin treatment being studied here is not currently available to individuals diagnosed with depression. Therefore, outside of research, this procedure is not one that would be typically encountered during treatment protocols aimed at improving depression.

### Duration

The entire duration of the study is anticipated to be 12 months. The study anticipates subject recruitment, screening, enrollment, and treatment to last up to 8 months once the first subject is enrolled. The study anticipates that an additional 1-3 months are required to complete data analyses. The IRB protocol period requested is 12 months.

### Participant Population

Based on the statistical power calculations, the study is 30 total subjects to be enrolled in and complete the study. It is estimated in the subject enrollment and treatment funnel that 93% of the subjects fail to meet this threshold post enrollment and randomization. It is anticipated that attrition in the study may be up to 20%. Thus, it is anticipated to contact 500-600 prospective subjects and enroll 36-40 subjects to gain 30 complete, protocol complaint subjects. The age range of subjects is 25-65 years old.

Inclusion criteria listed below is accompanied with an electronically administered inclusion/exclusion criteria checklist to be reviewed with prospective subjects during Office Visit #1 before measuring EKG and just prior to providing Informed Consent (see accompanying Medical Screen Form and Inclusion Exclusion Checklist Document). In addition to the inclusion and exclusion criteria listed below, subjects are given a Medical Screening Questionnaire to further confirm they are Medically Stable and the absence of medically excluding criteria.

Inclusion Criteria Includes:
Medically stable, adult males and females aged 25-65;
Capable of giving informed consent;
Who can reasonably meet the required study time commitment to complete the study;
Who meet all exclusions criteria or are not excluded for a medical condition identified during eligibility screening.
Who have been suffering from depression and anxiety symptoms for more than 6-weeks and have a BDI score >=20.

Exclusion Criteria Includes:
Has signs or symptoms of COVID-19. Subjects must be free of COVID symptoms before enrolling in the trial.
Currently (within the past 6 weeks) taking medication for the treatment of depression or anxiety;
Diagnosed with or suffering from schizophrenia, schizoaffective disorder, bipolar disorder, mania, or borderline personality disorder;
Any signs or symptoms of a neuropsychiatric disorder or disorganized thought processes that may be deemed by the study physician or staff as a disqualifying symptom;
Have a first generation relative (parent, sibling, child) that has been diagnosed with schizophrenia or schizoaffective disorder;
Concurrent or recent (within 30 days) use of any pharmacological or biologic agent for treating depression or anxiety (e.g., magnesium supplements, marijuana, etc);
Concurrent enrollment in CBT or psychotherapy for the treatment of depression or anxiety;
Participant is currently participating in or were discontinued within the last 30 days from another clinical trial involving an investigational drug or device for the treatment of depression or anxiety;
Any serious medical disorder or illness other than depression and anxiety;
Medically unstable;
Completion or enrollment of any drug or alcohol treatment program within the past 3 months;
History of drinking more than 12 alcohol drinks per week or 2 drinks per day within the past 3 months;
Subjects must refrain from alcohol use the day before and after treatment days during the study;
History of heart disease or cardiovascular disease;
Presence of abnormal EKG (ECG);
Recent hospitalization for surgery/illness (within the last 3 months);
History of hospitalization for neurological or psychological disorder (within the past 12 months);
Currently undergoing treatment or medication for neurological or psychological disorder, such as ADHD including addiction for less than 3 months;
History of migraines or frequent headaches (>2 per week);
History of panic attack or acute anxiety disorder that involves fainting;
History of more than 3 concussions or a traumatic brain injury;
Pregnant, breastfeeding or nursing (lactating) women;

### Risks and Benefits

The study involves the use of deception only in that it is a double-blinded study and the sham treatment is intended to be masked as an active treatment. Neither subjects or study personnel are able to identify the study conditions. No other form of deception is used in this study as trust between the study personnel and subjects is critical.

Subjects are assigned a participant Subject ID at the beginning of their enrollment to maintain strict confidentiality. Some surveys ask about current and past alcohol and drug use, as well as mental health and medical history. All this information is de-identified and coded using procedures as described to protect the identity or the subject and any data they provide to the study. All data are de-identified and remain in that format perpetually or for the life storage of the data. Confidentially protocols and procedures for protecting any and all sensitive data are clearly explained to prospective participants during the informed consent procedure.

Psilocybin is a hallucinogenic/psychedelic drug. At the higher doses, psilocybin can cause severe distortion in all body sensations and thinking. This can include abnormal perception in all senses such as visual changes, visual hallucinations, hearing changes and auditory hallucinations, abnormal smells or bodily sensation, and an unusual mixing of sensations where for example sounds may be perceived as pictures or images or colors could be perceived as tastes. These experiences are usually accompanied by intense changes in mood states. This can include elation or euphoria, anxiety and panic feelings, or depressive feelings. Emotions can change quickly from one to another. While these experiences are described by most people as pleasant or profound, to some it may be frightening and include symptoms of panic, depression, and confusion. The symptoms listed above usually begin within the first hour after taking the psilocybin and can last for up to 12 hours, although most people find that these symptoms are gone by 6 hours after ingestion. The maximum dose given in this study is about half of the dose where people report strong psychedelic effects.

Psilocybin can also cause dizziness, nausea, vomiting, headaches, increased pulse and blood pressure, dilated pupils, slightly elevated temperature, raising of skin-hair, and increased reflexes. These symptoms usually begin 20 to 30 minutes after taking the drug and can last up to 6 hours.

At times psychedelic sensations or memories of these sensations may be re-experienced in the future and this has been called “flashbacks”. It is not known how often this really occurs. Some studies have reported that it does not really happen and other studies suggest that some people do have these experiences. Other complications such as prolonged psychosis, suicide, or homicide have been attributed to use of hallucinogenic drugs. It is unclear to what extent these effects can be caused by hallucinogenic drugs.

The majority of side effects reported in the literature have occurred at doses of 20-30 mg (0.30-0.45 mg/kg). About half of the subjects experience some negative emotions or acute psychological distress at psilocybin doses of 30 mg and higher. At lower doses, the incidence of psychological stress and negative emotions are significantly reduced.

The highest dose used in this study is ˜10 mg (0.15 mg/kg) and as low as ˜1 mg (0.015 mg/kg). The threshold reported in the literature where mild hallucinogenic activity begins to occur in about half of the subjects is 10 mg psilocybin. It has been reported that subjects have significant difficulty distinguishing between active psilocybin and niacin at doses of 5 mg or less. These dose ranges are selected intentionally to avoid any strong emotional reactions or hallucinogenic activity. Some subjects may experience fluctuations in mood, psychophysiological arousal, or sensory/perceptual activity.

The study creates a safe, comfortable, and private research environment. Ensuring subjects are comfortable and relaxed during treatments is the most critical factor in minimizing and reducing negative side effects that have been reported using higher doses of psilocybin.

If any adverse reactions that lead the study doctors involved with this study to be concerned about the safety of a subject then subjects may be detained in the clinic until they feel safe to go home. If subjects that have a reaction such that they become very upset, it is possible that the study doctors may provide diazepam to help subjects calm down.

### Risk Analysis of EEG Recordings

Skin Irritation Risk. Minor skin irritation can occur (2-3%) if electrodes are placed over broken, irritated, or wounded skin. Placing electrodes over these areas is avoided. Moreover, participants visually inspect skin prior to electrode placement to further reduce the potential for minor irritation. Mild redness has been reported at the site of electrode placements, but this is an acute effect of vasodilation as opposed to inflammation and thus does not indicate damage.

Headache. A rare side effect of EEG recording is the occurrence of a mild headache or discomfort due to the headcap. This occurs in less than about 1-2% of subjects at incidence. Mild headaches typically resolve rapidly upon headcap adjustment or within a couple hours with no further complications. If a subject experiences a headache or discomfort, they may discontinue use at any time.

The risks of occurrence of the above adverse reactions have been minimized through Proper Instructions for Use documentation, training of Human Research Subjects on proper Study Device use, and in the usage and design of biocompatible electrode materials for device construction.

### Risk Avoidance

Two office visits are spent educating and informing research subjects about the risks associated with using hallucinogens, such as psilocybin. A major objective of the first two office visits is to acclimate subjects with the study site, study personnel, and study environment. The study creates a comfortable, relaxing, safe, and private environment that resembles a small apartment living atmosphere to help ensure subjects are completely comfortable in the treatment environment. High pre-treatment anxiety or stress can be a major confound in this study and can lead to increased incidence of negative emotions experienced when being treated with psychedelic compounds.

A minimum of four hours is spent educating patients using written materials and videos regarding the use of psilocybin in mental health research. Subjects are educated on the mechanisms of action, as well as dosage strategies and regimes based on the current literature. Subjects are educated on the potential side-effects of psilocybin. A major component of the education and subject preparation in the first two office visits is focused on acclimating subjects to the treatment procedures. The treatment and testing procedures allowing subjects to wear EEG headcaps, listen to auditory stimuli, and practice the Flanker and CPT tasks is discussed. Subjects are also able to request that certain drinks or food be present for refreshment during their treatment days. By the end of the second office visit and before the first treatment is scheduled for office visit 3, subjects should demonstrate they are comfortable with the testing and treatment protocols and procedures.

During the training and education to occur in the first two office visits, study personnel monitor the psychological state of subjects. If any disordered thought processes or neuropsychiatric symptoms are uncovered in this time spent with subjects then the study PI and Study Physician are notified. The presence of disordered or delusional thinking may cause increased risks of negative emotions or reactions to hallucinogens. Thus, continuous subject evaluation of behavioral disposition and psychological stability (fragility) during the training and education is intended to further identify subjects that may be at higher risks for experiencing negative symptoms or emotions during the treatment. A decision may be made by the Study Physician to exclude any subject from further study if a psychological risk factor is uncovered through interactions during the education and training office visits or any anytime thereafter in the study.

### Study Results

The results of the study help to validate basic scientific hypotheses regarding actions of psilocybin on sensory processing that low, sub-hallucinogenic doses of psilocybin can induce plasticity of brain activity in a manner that correlates with mood enhancement. For psilocybin and other classic psychedelics to provide scalable mental health therapies, the hallucinogenic activity must be reduced or minimized. Therefore, the research results obtained in this study will inform subsequent drug research development and formulation studies. These research results will ultimately lead to new drug formulations, which will be clinically evaluated for efficacy in treating depression, anxiety, and other mental health conditions.

Research subjects are introduced and educated on potentially new therapies involving psychedelics that can significantly improve mental health and quality of life. The study is justified by the potential gain of a viable and effective new therapy for the treatment of depression and stress disorders—a global health concern with few patient satisfactory treatment options that do not require use of other drugs that often have undesirable side effects.

### Example 5: Identifying High and Low Responders

Experiments were conducted to test psilocybin doses in the range of 0.05-0.2 mg/kg psilocybin administered S.C. across progressive ratio (PR) and 5-choice serial reaction time task (5CSRTT) to examine the effect of psilocybin on endophenotypes of motivation and attention. In each study, a population of out-bred Long Evans rats was first tested as a group for response to the PR and 5CSRTT. Each group was then divided into sub-groups by tertiles according to performance, and then exposed to various dose levels immediately prior to re-testing at various psilocybin dose levels. As explained below, rats in the lowest tertile sub-group displayed different responses than rats in the highest tertile.

The PR test is used to answer how willing the test subject is to work for food (i.e. test of motivation). A single 45 mg food pellet (i.e. reinforcer) is made available to the test animal based on lever press response. To obtain each successive pellet, the animal must make increasingly more lever presses. Typically, a progression of 2, 4, 6, 9, 12, 15, 20, 25, 32, 40, 50, 62, 77, 95, 118, etc. is used, derived from the equation:

ratio=[5×e^{(0.2×reinforcer#)}−5]

Hungry test animals do not find 45 mg food pellets sufficient and, therefore, there is a drive to repeatedly lever press to obtain multiple food pellets. At some point, the animal gives up as the motor demands to obtain a single pellet are not deemed worthwhile (i.e. animal reaches“break point” defined as animal's failure to earn a food pellet in 20 minutes).

Psilocybin was administered to rats S.C. at doses of 0.05, 0.1 and 0.2 mg/kg. None of these doses were observed to cause obvious changes in the number of lever presses or break points (i.e. rewards earned) when studied across entire study population of 36 rats.

Rats were then sub-grouped into tertiles based on the number of lever presses for food at baseline, so that within the test population of 36 animals, 12 animals were identified as low responders, characterized by low motivation and potentially corresponding to low motivation endophenotype representative of clinical depression. Twelve animals were identified as high responders. There was a statistically significant difference between the high and low responders. Psilocybin doses of 0.05-0.2 mg/kg administered S.C. were observed to produce no effect on response in high responder subgroup (FIG. 22). However, 0.05 mg/kg psilocybin was observed to produce an increase in food responding in low responder subgroup, whereas the dose of 0.1 mg/kg did not produce an increase in food responding. This represents a motivational enhancing effect of psilocybin in the cohort corresponding to a low motivation endophenotype representative of clinical depression.

The 5CSRTT involves evaluating test subject response to a brief visual stimulus (Higgins, Guy & Silenieks, Leonardo. (2017). Rodent Test of Attention and Impulsivity: The 5-Choice Serial Reaction Time Task: The 5-Choice Serial Reaction Time Task. 10.1002/cpph.27). Animals are trained to make a nose-poke response to a stimulus location in order to collect a food reward. The task allows the experimenter to measure animal performance in multiple domains including:
Attention
Impulsivity
Perseveration
Speed of response

A strength of the test is its flexible configuration to challenge test subjects:
Standard test conditions (0.75s stimulus duration (SD), 5s inter-trial interval (ITI), 100 trials)
Multiple short stimulus duration (mSD) (0.03-1s SD)
Fixed long ITI (5 s vs. 10 s ITI)
Multiple ultrashort ITI (2-5s ITI)
Extended 250 trials

Psilocybin dose of 0.05 mg/kg administered S.C. was observed to produce an increased (p=0.05, t-test) pro-cognitive effect (measured as % hit, calculated as #correct/(#correct+#incorrect+#omissions)*100) when studied across a study population of 24 rats in a 5CSRTT with standard conditions, i.e. 75s SD, 5S ITI, 100 trials. Asterisk (*) indicates statistical significance vs. vehicle. (FIG. 23A). There was a slight non-significant increase in procognitive effect (measured as % correct, calculated as #correct/(#correct+#incorrect)*100 (FIG. 23B). There was no effect on performance, e.g no effect on response speed, or number of trials completed. There was no effect on premature or perseverative responses in this experiment.

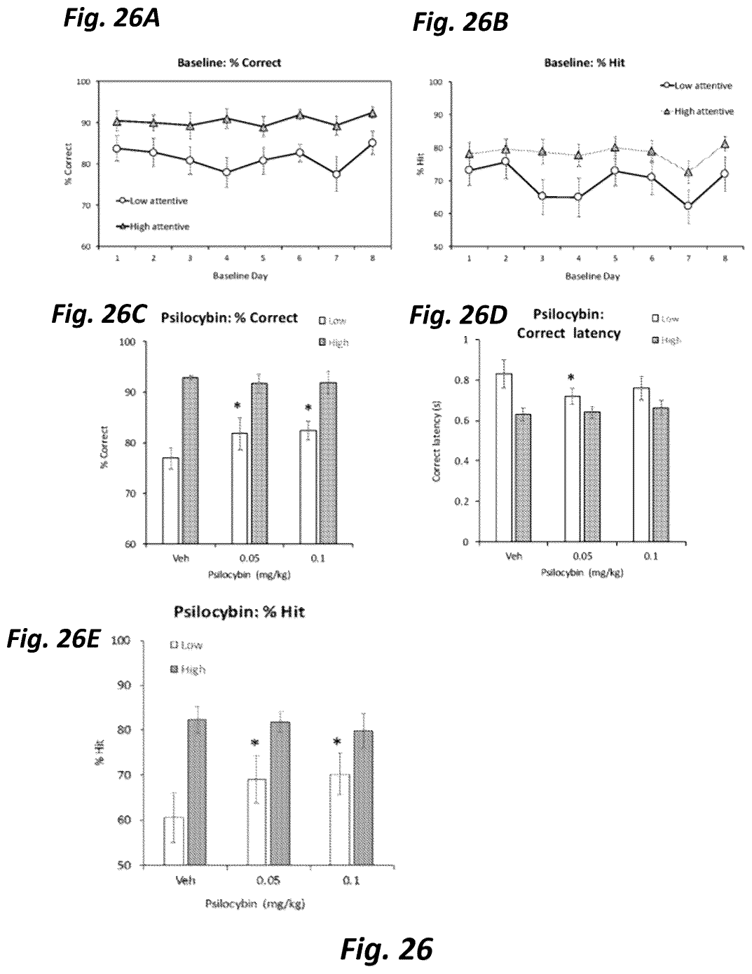

When this population is segmented into tertiles according to performance based on accuracy (% correct, calculated as #correct/(#correct+#incorrect)*100), the lowest performing tertile (N=8) are considered to be low attentive and potentially representative of a low attentive endophenotype of depression (FIG. 26A). Low attentive rats also score poorly on % hit (FIG. 26B) and have a slower response speed (FIG. 26D). Similar to the PR test, the effect of 0.05 and 0.1 mg/kg psilocybin on accuracy (% correct and % hit) in the 5CSRTT was observed to be strongly evident in the low attentive subgroup compared with vehicle (FIGS. 26C and 26E). Asterisk (*) indicates statistical significance vs. vehicle. Psilocybin 0.05 mg/kg also increased response speed in the low attentive cohort compared with vehicle (FIG. 26D). Using a longer duration between stimulus and reward, the 5CSRTT study measures premature (PREM) and perseverant (PSV) responses. PREM/PSV responses were increased by increasing the ITI from 5s (baseline) to 10s (test condition). A psilocybin dose of 0.05 mg/kg administered S.C. was observed to produce an increase (p=0.05, t-test) increase in PREM and PSV responses under a 10s ITI when studied across a study population of 24 rats (FIG. 24A). Low responders (N=8) were observed to improve significantly from psilocybin administration at both 0.05 and 0.1 mg/kg doses (FIG. 24B, p<0.01, t-test), with marked increases in premature responses and perseverative responses in rats in that sub-group compared with vehicle. Both PREM and PSV behaviors are examples of executive cognitive function, likely involving areas of the prefrontal cortex, a brain region rich in 5-HT2A receptors.

At doses that did not produce effects in animals indicative of hallucination, improved results were seen on low performing animals on measures of motivation, attention, accuracy, speed of response, perseveration, and cognitive engagement. The improvement in the low performing animals indicates utility of non-hallucinogenic doses of psilocybin in treatment of behavioral and cognitive disorders involving these behaviors, including but not limited to depression, anxiety, apathy and low motivation, attention disorders, disorders of executive function and cognitive engagement, obsessive compulsive disorder, and neurocognitive disorders. At these same doses, no detrimental effects of psilocybin were noted on performance, i.e. there was no evidence of reduced motivation, impaired motor control, or impaired attention or response speed. The positive effects of the low doses of psilocybin appear most evident in the low performer subgroups based on three tests: one PR (motivation) and two 5CSRTT (attention) studies were conducted, using psilocybin at 0.05-0.2 mg/kg (PR) and 0.05-0.1 mg/kg S.C. (5CSRTT). Significant improvements were noted in the low performing animals on:
Number of lever presses and increased break point in PR test (0.05 mg/kg)
% Correct and % Hit in 5CSRTT (0.05 and 0.1 mg/kg)
Increased speed of responding in 5CSRTT (0.05 mg/kg)
PREM/PSV in 5CSRTT 10s ITI (0.05 and 0.1 mg/kg

While preferred embodiments have been shown and described herein, it will be obvious to those skilled in the art that such embodiments are provided by way of example only. Numerous variations, changes, and substitutions will now occur to those skilled in the art without departing from this disclosure. It should be understood that various alternatives to the embodiments described herein might be employed in practicing current disclosure.

1-127. (canceled)

128. A computer-implemented method for identifying a therapeutically effective dose of a 5-HT receptor agonist administered to an individual, the method comprising:
a) identifying or measuring a first state of the individual by performing an evaluation on the individual, wherein the first state comprises one or more of (1) a physiological parameter, (2) a biological state, or (3) an emotional state;
b) administering the 5-HT receptor agonist to the individual;
c) identifying or measuring a second state of the individual by performing an evaluation on the individual, wherein the second state comprises one or more of (1) a physiological parameter, (2) a biological state, or (3) an emotional state subsequent to administering the 5-HT receptor agonist; and
d) determining the therapeutically effective dose of the 5-HT receptor agonist based at least in part on a comparison between the first and second states of the individual.

129. The computer-implemented method of claim 128, wherein the physiological parameter comprises one or more of a brain activity, blood pressure, a heart rate, or a breathing rate.

130. The computer-implemented method of claim 128, wherein identifying or measuring the biological state comprises evaluating a biological sample comprising one or more of serum, plasma, whole blood, urine, or sweat.

131. The computer-implemented method of claim 128, wherein the emotional state comprises one or more of a mood rating, a sleep rating, a coping rating, a stress rating, an anxiety rating, or a memory rating.

132. The computer-implemented method of claim 129, wherein the physiological parameter is identified or measured using at least one of an electroencephalogram (EEG), a magnetoencephalogram (MEG), functional near-infrared spectroscopy (fNIRS), PET transcranial functional ultrasound imaging, or a biosensor.

133. The computer-implemented method of claim 131, wherein the emotional state is identified or measured by administering to the individual one or more questionnaires and receiving the individual's responses to the one or more questionnaires.

134. The computer-implemented method of claim 128, wherein the first or second state is identified or measured using a wearable device or a mobile device.

135. The computer-implemented method of claim 128, wherein the therapeutically effective dose is effective in treating or managing a mental, a behavioral, or a neuropsychiatric condition, or the symptoms thereof, in the individual.

136. The computer-implemented method of claim 135, wherein treating or managing the mental, the behavioral, or the neuropsychiatric condition, or the symptoms thereof, comprises one or more of improving motivation or cognitive engagement in the individual.

137. The computer-implemented method of claim 135, wherein the condition is induced by one or more of stress or anxiety.

138. The computer-implemented method of claim 135, further comprising subjecting the individual to one or more auditory stimulus or one or more visual stimulus subsequent to step (b); and
wherein the first and second state comprise at least a physiological parameter comprising a brain activity, and wherein the second amount of the one or more 5-HT agonist is based at least in part on a change in brain activity between the first state and the second state.

139. The computer-implemented method of claim 135, wherein the condition is one or more of an attention condition, a cognitive condition, addiction, anxiety, apathy, or depression.

140. The computer-implemented method of claim 139, wherein the attention condition is attention deficit hyperactivity disorder (ADHD) or attention deficit disorder (ADD).

141. The computer-implemented method of claim 128, wherein the 5-HT receptor agonist, or a pharmaceutically acceptable salt, solvate, metabolite, derivative, or prodrug thereof, is administered to the individual in an amount of about 0.01 mg to about 100 mg.

142. The computer-implemented method of claim 128, wherein the second state is identified or measured at least twenty minutes or more after the first state is identified or measured.

143. The computer-implemented method of claim 128, further comprising transmitting the therapeutically effective dose of the 5-HT receptor agonist to the individual, a caregiver, or both.

144. The computer-implemented method of claim 128, wherein the 5-HT receptor agonist is administered to the individual in need thereof as an oral formulation, an intravenous formulation, a subcutaneous formulation, a dental formulation, a buccal formulation, a nasal formulation or an inhalation formulation.

145. The computer-implemented method of claim 128, wherein the therapeutically effective dose of the 5-HT receptor agonist is for treating brain inflammation or brain fog following insult in the individual.

146. A method for treating a mental, a behavioral, or a neuropsychiatric condition treatable with a 5-HT receptor agonist, or symptoms thereof, in an individual in need thereof, comprising:
a. identifying or measuring a first state of the individual by performing an evaluation on the individual, wherein the first state comprises one or more of (1) a physiological parameter, (2) a biological state, or (3) an emotional state;
b. administering to the individual a first amount of one or more 5-hydroxytryptamine (5-HT) receptor agonist, or a pharmaceutically acceptable salt, solvate, metabolite, derivative, or prodrug thereof;
c. identifying or measuring a second state of the individual by performing a second evaluation subsequent to administering the first amount of the one or more 5-HT receptor agonist, or a pharmaceutically acceptable salt, solvate, metabolite, derivative, or prodrug thereof, wherein the second state comprises one or more of (1) a physiological parameter, (2) a biological state, or (3) an emotional state; and
d. administering to the individual a second amount of one or more 5-HT receptor agonist, or a pharmaceutically acceptable salt, solvate, metabolite, derivative, or prodrug thereof, thereby treating or managing the mental, the behavioral, or the neuropsychiatric condition, or the symptoms thereof, in the individual.

147. The method of claim 146, further comprising:
subjecting the individual to one or more auditory stimulus or one or more visual stimulus subsequent to step (b); and
wherein the first and second state comprise at least a physiological parameter comprising a brain activity, and wherein the second amount of the one or more 5-HT agonist is based at least in part on a change in brain activity between the first state and the second state.
